(19) United States
(12) Patent Application Publication
(10) Pub. No.: US 2025/0368625 A1
(43) Pub. Date: Dec. 4, 2025

(54) PIPERAZINES

(71) Applicant: Grünenthal GmbH, Aachen (DE)

(72) Inventors: Sebastian PEIL, Aachen (DE); Sonja NORDHOFF, Aachen (DE); Clemens DIALER, Aachen (DE); Martin PETTERSSON, Aachen (DE)

(73) Assignee: Grünenthal GmbH, Aachen (DE)

(21) Appl. No.: 19/221,745

(22) Filed: May 29, 2025

Related U.S. Application Data
(60) Provisional application No. 63/652,755, filed on May 29, 2024.

(30) Foreign Application Priority Data
May 29, 2024 (EP) 24178741.5

(51) Int. Cl. C07D 403/12; A61K 31/496; C07D 413/12; C07D 471/04

(52) U.S. Cl. CPC C07D 403/12; A61K 31/496; C07D 413/12; C07D 471/04

(57) ABSTRACT

Compounds according to general formula (I)

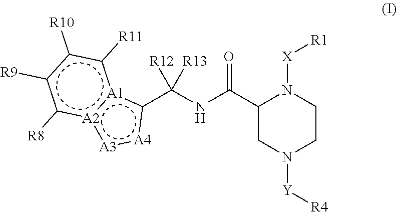

are modulators of the SSTR4 receptor and pharmaceutical compositions containing them are useful in the treatment of pain.

The present application claims priority of European Patent Application No. 24178741.5, filed May 29, 2024, and of U.S. Provisional Application No. 63/652,755, filed May 29, 2024, the entire contents of which patent applications are hereby incorporated herein by reference.

The invention relates to compounds according to general formula (I)

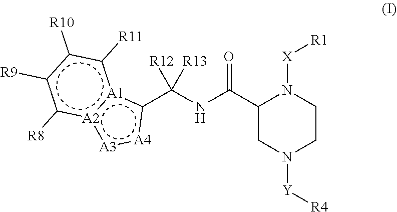

which are modulators of the SSTR4 receptor.

### BACKGROUND

Somatostatin (SST) is an endogenous peptide that inhibits the secretion of other hormones such as growth hormone, thyroid stimulating hormone, cholecystokinin and insulin (Reichlin, S. “Somatostatin,” New England Journal of Medicine, (1983) 309, 1495-1501). Somatostatin binds to five different receptor subtypes (SSTR1-5) that are all Gai coupled G-protein coupled receptors (GPCRs) but differ by function and expression. Preclinical data suggest the SSTR4 receptor subtype plays a role in modulating pain, and that agonism of this receptor may be analgesic.

SSTR4 is expressed in areas associated with pain processing such as dorsal root ganglia (DRG) neurons (Shenoy et. al., “The Somatostatin Receptor-4 Agonist J-2156 Alleviates Mechanical Hypersensitivity in a Rat Model of Breast Cancer Induced Bone Pain,” Frontiers in Pharmacology, (2018) 9(495)) and somatosensory cortical neurons (Kecskés et. al., “Characterization of Neurons Expressing the Novel Analgesic Drug Target Somatostatin Receptor 4 in Mouse and Human Brains,” International Journal of Molecular Sciences, (2020) 21(7788)).

In DRG neurons, agonism of SSTR4 has been shown to activate G-protein coupled inward rectifying potassium (GIRK) channels and inhibit voltage sensitive calcium currents (Gorham et. al., “Somatostatin 4 receptor activation modulates G-protein coupled inward rectifying potassium channels and voltage stimulated calcium signals in dorsal root ganglion neurons,” European Journal of Pharmacology, (2014) 736: 101-106).

Additionally, SSTR4 agonism increased a depolarization-activated, non-inactivating K⁺ current (M-current) and reduced the firing rate of layer V cortical pyramidal neurons (Kecskés et. al., “Characterization of Neurons Expressing the Novel Analgesic Drug Target Somatostatin Receptor 4 in Mouse and Human Brains,” International Journal of Molecular Sciences, (2020) 21(7788)) and hippocampal neurons (Qiu et. al., “Somatostatin Receptor Subtype 4 Couples to the M-Current to Regulate Seizures, “The Journal of Neuroscience,” (2008) 28(14):3567-3576).

Taken together, these data suggest SSTR4 agonism reduces neuronal excitability via modulation of ion channels and that the development of SSTR4 agonists may be useful as novel analgesics.

N-(2-(het)arylpropan-2-yl) acyl amides with SSTR4 activity are known from e.g. WO 2010 059922, WO 2014 184275, WO 2015 037716, WO 2016 075239, WO 2016 075240, WO 2021 233427, WO 2021 233428, WO 2021 202781, and WO 2022 012534.

In addition, related structures such as substituted isobutyramides with SSTR4 activity are known from e.g. WO 2019 169153 and WO 2023 187677.

Miscellaneous chemotypes such as (pseudo-)peptides and heteroaromatic compounds with SSTR4 activity are known from e.g. WO 2005 033069, WO 2005 033124, WO 2021 202775, and WO 2023 180125.

It is an object of the invention to provide novel compounds which are modulators of the SSTR4 receptor. The novel compounds should in particular be suitable for use in the treatment and/or prophylaxis of disorders or diseases which are at least partially mediated by the SSTR4 receptor.

This object has been achieved by the subject-matter of the patent claims.

A first aspect of the invention relates to a compound according to general formula (i)

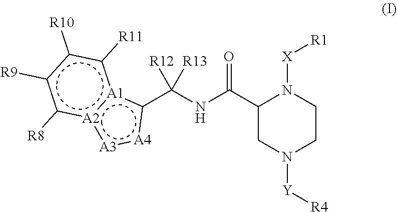

wherein
A1 represents C or N;
A2 represents C or N;
A3 represents CR7, N, NR7, or O;
A4 represents CH, or N;
with the proviso that at least one of A1, A2 and A4 represents N, or at least A3 represents N or NR7;
R1 and R4 independently of one another represent
   —H;
   —C₁₋₆-alkyl unsubstituted or substituted with one, two, three or four substituents independently of one another selected from —F, —OH, —OCH₃ and —OCF₃;
   —C₃₋₈-cycloalkyl, unsubstituted or substituted with one, two, three or four substituents independently of one another selected from —F, —OH, —C₁₋₆-alkyl and —O—C₁₋₆-alkyl, wherein in each case alkyl is unsubstituted or substituted with one, two or three —F;
   —C₂₋₇-heterocycloalkyl, unsubstituted or substituted with one, two, three or four substituents independently of one another selected from —F, —OH, —C₁₋₆-alkyl and —O—C₁₋₆-alkyl, wherein in each case alkyl is unsubstituted or substituted with one, two or three —F;
      -aryl, unsubstituted or substituted with one, two, three or four substituents independently of one another selected from —F, —Cl, —OH, —C₁₋₆-alkyl and —O—C₁₋₆-alkyl, wherein in each case alkyl is unsubstituted or substituted with one, two or three —F; or
      -heteroaryl, unsubstituted or substituted with one, two, three or four substituents independently of one another selected from —F, —Cl, —OH, -cyclopropyl unsubstituted, —C₁₋₆alkyl and —O—C₁₋₆-alkyl, wherein in each case alkyl is unsubstituted or substituted with one, two or three —F;
X represents a bond, —C(═O)— or —CR2R3-;
Y represents a bond, —C(═O)—, —C(═O)CH₂—, or —CR5R6-;
R2, R3, R5 and R6 independently of one another represent
   —H; or
   —C₁₋₆-alkyl unsubstituted or substituted with one, two, three or four substituents independently of one another selected from —F, —OH, and —OCH₃;
R7 represents
   —H;
   —F or —Cl;
   —C₁₋₆-alkyl or —O—C₁₋₆-alkyl, wherein in each case alkyl is unsubstituted or substituted with one, two or three —F; or
   —C₃₋₈-cycloalkyl, —O—C₃₋₈-cycloalkyl, or —CH₂—C₃₋₈-cycloalkyl, wherein in each case cycloalkyl is unsubstituted or substituted with one, two or three —F;
with the proviso that when A3 represents NR7, R7 represents
   —H;
   —C₁₋₆-alkyl unsubstituted or substituted with one, two or three —F; or
   —C₃₋₈-cycloalkyl or —CH₂—C₃₋₈-cycloalkyl, wherein in each case cycloalkyl is unsubstituted or substituted with one, two or three —F;
R8 represents
   —H;
   —F or —Cl;
   —C₁₋₆-alkyl or —O—C₁₋₆-alkyl, wherein in each case alkyl is unsubstituted or substituted with one, two, three or four substituents independently of one another selected from —F, —OH, and —OCH₃; or
   —C₃₋₄-cycloalkyl or —CH₂—C₃₋₄-cycloalkyl, wherein in each case cycloalkyl is unsubstituted or substituted with one, two, three or four substituents independently of one another selected from —F, —OH, —OCH₃, and —C₁₋₆-alkyl unsubstituted or substituted with one, two or three —F;
R9, R10 and R11 independently of one another represent —H or —F;
R12 and R13 independently of one another represent —H or —C₁₋₆-alkyl unsubstituted or substituted with one, two or three —F; or R12 and R13 together with the carbon atom to which they are attached form C₃₋₆-cycloalkyl unsubstituted or substituted with one, two or three —F;
or a physiologically acceptable salt thereof.

In a preferred embodiment, the compound according to the invention is present in form of the free compound. For the purpose of specification, “free compound” preferably means that the compound according to the invention is not present in form of a salt. Methods to determine whether a chemical substance is present as the free compound or as a salt are known to the skilled artisan such as ¹⁴N or ¹⁵N solid state NMR, x-ray diffraction, x-ray powder diffraction, IR, Raman, XPS. ¹H-NMR recorded in solution may also be used to consider the presence of protonation.

In another preferred embodiment, the compound according to the invention is present in form of a physiologically acceptable salt. For the purposes of this specification, the term “physiologically acceptable salt” preferably refers to a salt obtained from a compound according to the invention and a physiologically acceptable acid or base.

According to the invention, the compound according to the invention may be present in any possible form including solvates, cocrystals and polymorphs. For the purposes of this specification, the term “solvate” preferably refers to an adduct of (i) a compound according to the invention and/or a physiologically acceptable salt thereof with (ii) distinct molecular equivalents of one or more solvents.

Further, the compound according to the invention may be present in form of the racemate, enantiomers, diastereomers, tautomers or any mixtures thereof.

The compounds according to the invention may have one or more stereocenters. The person skilled in art knows by looking at a chemical structure whether the depicted compound has one or more stereocenters or not.

For some compounds according to the invention that have one or more stereocenters and which chemical structures are disclosed in the examples of the present application, the chemical structure includes bold bonds and/or hashed bonds to indicate the relative structural orientation of those substituents connected by the bold bonds and/or hashed bonds to the superior structure. If the bold bonds and/or hashed bonds are depicted in form of a wedge, the absolute stereochemical configuration of the compound is known and thereby indicated. If the bold bonds and/or hashed bonds are depicted as a straight bond (i.e. no wedge), the absolute stereochemical configuration of the compound has not been determined. In that case, the bold bonds and/or hashed bonds merely serve to indicate that this particular compound is present as one enantiomer or one diastereomer (e.g. cis-diastereomer (i.e. mixture of two cis-enantiomers) or trans-diastereomer (i.e. mixture of two trans-enantiomers)). All compounds according to the invention that have one or more stereocenters but which chemical structures disclosed in the examples of the present application do not include bold bonds and/or hashed bonds, are present as a mixture of the respective stereoisomers.

The invention also includes isotopic isomers of a compound of the invention, wherein at least one atom of the compound is replaced by an isotope of the respective atom which is different from the naturally predominantly occurring isotope, as well as any mixtures of isotopic isomers of such a compound. Preferred isotopes are ²H (deuterium), ³H (tritium), ¹³C and ¹⁴C. Isotopic isomers of a compound of the invention can generally be prepared by conventional procedures known to a person skilled in the art.

According to the invention, the term “C₁₋₆-alkyl” preferably means acyclic and preferably saturated hydrocarbon residues, which can be linear (i.e. unbranched) or branched, and which contain 1 to 6 (i.e. 1, 2, 3, 4, 5 or 6) carbon atoms. Preferably, C₁₋₆-alkyl is saturated.

Preferred C₁₋₆-alkyl groups are selected from the group consisting of methyl, ethyl, n-propyl, 2-propyl, n-butyl, isobutyl, sec-butyl, tert-butyl, n-pentyl, 2-pentyl, 3-pentyl, 2-methylbutyl, 3-methylbutyl, 3-methylbut-2-yl, 2-methylbut-2-yl, n-hexyl, 2-hexyl, 3-hexyl, 2-methylpentyl, 4-methylpentyl, 4-methylpent-2-yl, 2-methylpent-2-yl, 3,3-dimethylbutyl, 3,3-dimethylbut-2-yl, 3-methylpentyl, 3-methylpent-2-yl and 3-methylpent-3-yl; more preferably methyl, ethyl, n-propyl, 2-propyl, n-butyl, isobutyl, sec-butyl, tert-butyl, n-pentyl, 2-pentyl, 3-pentyl, 2-methylbutyl, 3-methylbutyl, 3-methylbut-2-yl, 2-methylbut-2-yl, 2,2-dimethylpropyl, n-hexyl. Particularly preferred C₁₋₆-alkyl groups are selected from C₁₋₄-alkyl groups. Preferred C₁₋₄-alkyl groups are selected from the group consisting of methyl, ethyl, n-propyl, 2-propyl, n-butyl, isobutyl, sec-butyl and tert-butyl.

According to the invention, the terms “C₃₋₈-cycloalkyl” and “C₃₋₄-cycloalkyl” preferably mean monocyclic aliphatic hydrocarbons containing 3, 4, 5, 6, 7, or 8 carbon atoms as ring members and 3 or 4 carbon atoms as ring members, respectively, wherein the hydrocarbons in each case can be saturated or unsaturated (but not aromatic). Preferably, C₃₋₈-cycloalkyl and C₃₋₄-cycloalkyl are saturated. The C₃₋₈-cycloalkyl and C₃₋₄-cycloalkyl can be bound to the respective superordinate general structure via any desired and possible ring member of the cycloalkyl group. The C₃₋₈-cycloalkyl and C₃₋₄-cycloalkyl are not condensed with further ring systems and are not bridged.

Preferred C₃₋₈-cycloalkyl groups are selected from the group consisting of cyclopropyl, cyclobutyl, cyclopentyl, cyclohexyl, cycloheptyl, and cyclooctyl. Preferred C₃₋₈-cycloalkyl groups are selected from C₃₋₆-cycloalkyl groups.

According to the invention, the term “C₂₋₇-heterocycloalkyl” preferably means monocyclic, heterocycloaliphatic saturated or unsaturated (but not aromatic) residues having 3 to 8, i.e. 3, 4, 5, 6, 7, or 8 ring members, wherein in each case at least one, if appropriate also two or three ring members, are a heteroatom or a heteroatom group independently one another selected from the group consisting of O, S, S(═O), S(═O)₂, N, NH and N(C₁₋₄-alkyl) such as N(CH₃). Preferably, the C₂₋₇-heterocycloalkyl contains only one heteroatom or heteroatom group within the ring. Preferably, C₂₋₇-heterocycloalkyl are saturated. The C₂₋₇-heterocycloalkyl are not condensed with further ring systems and are not bridged. The C₂₋₇-heterocycloalkyl can be bound to the superordinate general structure via any desired and possible ring member of the heterocycloaliphatic residue if not indicated otherwise. In a preferred embodiment, C₂₋₇-heterocycloalkyl are bound to the superordinate general structure via a carbon atom.

Preferred C₂₋₇-heterocycloalkyl are selected from the group consisting of oxetanyl, tetrahydrofuranyl, tetrahydropyranyl, morpholinyl, thiomorpholinyl, tetrahydrothiopyranyl, tetrahydrothiopyranyl 1,1-dioxide, oxepanyl, piperidinyl, piperidinonyl, azetidinyl, pyrrolidinyl, pyrrolidinonyl, 4-methylpiperazinyl, morpholinonyl, dioxanyl, piperazinyl, tetrahydropyrrolyl, azepanyl, dioxepanyl, oxazepanyl, diazepanyl, thiazolidinyl, tetrahydrothiophenyl, tetrahydropyridinyl, dithiolanyl, dihydropyrrolyl, dioxolanyl, dihydropyridinyl, dihydrofuranyl, dihydroisoxazolyl, dihydrooxazolyl, imidazolidinyl, isoxazolidinyl, oxazolidinyl, piperazinyl, N-methylpyridinonyl, pyrazolidinyl, pyranyl; dihydroquinolinyl, dihydroisoquinolinyl, dihydroindolinyl, dihydroisoindolyl, tetrahydroquinolinyl, tetrahydroisoquinolinyl and tetrahydroindolinyl; more preferably oxetanyl, tetrahydrofuranyl, tetrahydropyranyl, morpholinyl, thiomorpholinyl, tetrahydrothiopyranyl, tetrahydrothiopyranyl 1,1-dioxide, oxepanyl, piperidinyl, piperidinonyl, azetidinyl, pyrrolidinyl, pyrrolidinonyl, 4-methylpiperazinyl, morpholinonyl, dioxanyl, piperazinyl, and tetrahydropyrrolyl.

According to the invention, the term “aryl” preferably refers to phenyl or naphthyl, more preferably phenyl.

According to the invention, the term “heteroaryl” preferably refers to a 5- to 10-membered heteroaryl preferably meaning a 5 to 10-membered cyclic aromatic residue containing at least 1, if appropriate also 2, 3, 4 or 5 heteroatoms, wherein the heteroatoms are each selected independently of one another from the group S, N and O. The binding to the superordinate general structure can be carried out via any desired and possible ring member of the heteroaryl residue if not indicated otherwise. Preferably, the heteroaryl is bound to the superordinate general structure via a carbon atom of the heterocycle. The heteroaryl can also be condensed with a further ring system, e.g. saturated or (partially) unsaturated (hetero)cyclic, aromatic or heteroaromatic ring systems.

Preferably, the heteroaryl is selected from the group consisting of pyridyl (i.e. 2-pyridyl, 3-pyridyl, 4-pyridyl), pyrazolyl, pyrrolo[2,3-b]pyridyl, pyridonyl (pyridinonyl), thienyl (thiophenyl), thiazolyl, 2,3-dihydrobenzo[d]isothiazolyl 1,1-dioxide, isoindolinonyl, pyrimidinyl, pyrazinyl, pyridazinyl, isoxazolyl, triazolyl, oxazolyl, oxadiazolyl, indazolyl, pyrazolopyridyl, pyrrolyl, imidazolyl, isothiazolyl, furanyl, thiadiazolyl, N-methylpyridinonyl, benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, imidazothiazolyl, indolizinyl, indolyl, isoquinolinyl, naphthyridinyl, phenazinyl, phenothiazinyl, phthalazinyl, purinyl, phenazinyl, tetrazolyl and triazinyl; more preferably pyridyl, pyrazolyl, pyrrolo[2,3-b]pyridyl, pyridonyl, thienyl, thiazolyl, 2,3-dihydrobenzo[d]isothiazolyl 1,1-dioxide, isoindolinonyl, pyrimidinyl, pyrazinyl, pyridazinyl, isoxazolyl, triazolyl, oxazolyl, oxadiazolyl, indazolyl, pyrazolopyridyl, pyrrolyl, imidazolyl, isothiazolyl, furanyl, and thiadiazolyl.

In preferred embodiments, the compound according to the invention is according to any one of general formulas (II-a) to (II-h):

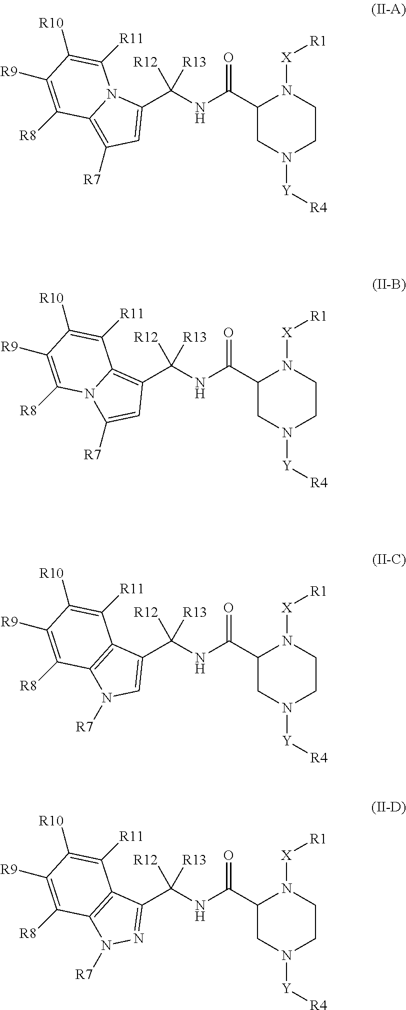

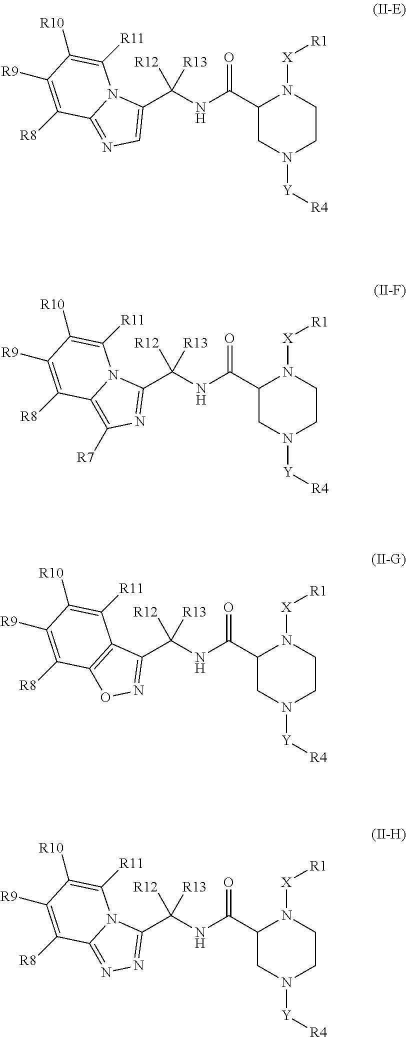

Preferably, X represents a bond, —C(═O)—, —CH₂—, or —CH(CH₃)—. When X represents a bond, R1 is directly bonded to the N atom of the piperazine moiety.

Preferably, R1 represents
—H;
—CH₃, —CH₂CH₃, —CH(CH₃)₂, —CH₂CH(CH₃)₂, —CH₂CHF₂, —CH₂CF₃, —CH₂CF(CH₃)₂, —CH₂CF₂CH₃, —CH₂CH₂OCH₃, —CH₂C(CH₃)₂OH, or —CH₂C(CH₃)₂CH₂OCH₃;
-cyclopropyl, -cyclobutyl, or -cyclohexyl; in each case unsubstituted or substituted with one or two substituents independently of one another selected from —F and —CF₃;
-oxetanyl or -tetrahydropyranyl; in each case unsubstituted or monosubstituted with —CH₃;
-phenyl unsubstituted or monosubstituted with —F or —CF₃; or
-pyrazolyl, -pyridinyl, -pyrimidinyl, -pyridazinyl, or -pyrazinyl; in each case unsubstituted or monosubstituted with —CH₃, —CF₃, or -cyclopropyl unsubstituted.

Preferably, Y represents a bond, —C(═O)—, —C(═O)CH₂—, —CH₂—, or —CH(CH₃)—. When Y represents a bond, R4 is directly bonded to the N atom of the piperazine moiety.

Preferably, R4 represents
—H;
—CH₃, —CH₂CH₃, —CH₂CH₂CH₃, —CH(CH₃)₂, —CH₂CH(CH₃)₂, —CH₂CHF₂, —CH₂CF₃, or —CH₂CH₂OCH₃;
-phenyl unsubstituted;
-cyclopropyl, cyclobutyl or cyclohexyl; in each case unsubstituted or substituted with one or two —F;
-oxetanyl or -tetrahydropyranyl; in each case unsubstituted; or
-pyrazolyl, -oxazolyl, -pyridyl; in each case unsubstituted or monosubstituted with —CH₃, or —CH(CH₃)₂.

Preferably, R7 represents —H or —CH₃.

Preferably, R8 represents —H, —F, —Cl, —CH₃, —CF₃, —OCH₃, or -cyclopropyl unsubstituted.

Preferably, R9, R10 and R11 each represent —H.

Preferably, R12 and R13 each represent —CH₃.

Preferably, the compound according to the invention is selected from the group consisting of

| | |
|---|---|
| 001 | rac-N-(2-(8-methylimidazo[1,5-a]pyridin-3-yl)propan-2-yl)-1-(pyrimidin-2- |
| | ylmethyl)piperazine-2-carboxamide |
| 002 | rac-1-isopropyl-N-(2-(8-methylimidazo[1,5-a]pyridin-3-yl)propan-2-yl)piperazine- |
| | 2-carboxamide |
| 003 | rac-N-(2-(8-methylimidazo[1,5-a]pyridin-3-yl)propan-2-yl)-1-(pyridin-2- |
| | ylmethyl)piperazine-2-carboxamide |
| 004 | rac-N-(2-(8-methylimidazo[1,5-a]pyridin-3-yl)propan-2-yl)-1-(pyridazin-3- |
| | ylmethyl)piperazine-2-carboxamide |
| 005 | rac-N-(2-(8-methylimidazo[1,5-a]pyridin-3-yl)propan-2-yl)-1-(pyrimidin-5- |
| | ylmethyl)piperazine-2-carboxamide |
| 006 | rac-1-((1-methyl-1H-pyrazol-3-yl)methyl)-N-(2-(8-methylimidazo[1,5-a]pyridin-3- |
| | yl)propan-2-yl)piperazine-2-carboxamide |
| 007 | rac-N-(2-(8-methylimidazo[1,5-a]pyridin-3-yl)propan-2-yl)-1-(pyrazin-2- |
| | ylmethyl)piperazine-2-carboxamide |
| 008 | rac-N-(2-(8-methylimidazo[1,5-a]pyridin-3-yl)propan-2-yl)-1-(pyrimidin-4- |
| | ylmethyl)piperazine-2-carboxamide |
| 009 | rac-1-acetyl-N-(2-(8-methylimidazo[1,5-a]pyridin-3-yl)propan-2-yl)piperazine-2- |
| | carboxamide |
| 010 | rac-1-(1-methyl-1H-pyrazole-4-carbonyl)-N-(2-(8-methylimidazo[1,5-a]pyridin-3- |
| | yl)propan-2-yl)piperazine-2-carboxamide |
| 011 | rac-1-(3,3-difluorocyclobutane-1-carbonyl)-N-(2-(8-methylimidazo[1,5-a]pyridin- |
| | 3-yl)propan-2-yl)piperazine-2-carboxamide |
| 012 | rac-1-(cyclopropanecarbonyl)-N-(2-(8-methylimidazo[1,5-a]pyridin-3-yl)propan-2- |
| | yl)piperazine-2-carboxamide |
| 013 | rac-N-(2-(8-methylimidazo[1,5-a]pyridin-3-yl)propan-2-yl)-1-(oxetane-3- |
| | carbonyl)piperazine-2-carboxamide |
| 014 | rac-N-(2-(8-methylimidazo[1,5-a]pyridin-3-yl)propan-2-yl)-1-(oxetan-3- |
| | ylmethyl)piperazine-2-carboxamide |
| 015 | (S)-N-(2-(8-methylimidazo[1,5-a]pyridin-3-yl)propan-2-yl)piperazine-2-carboxamide |
| 016 | (S)-1-methyl-N-(2-(8-methylimidazo[1,5-a]pyridin-3-yl)propan-2-yl)piperazine-2- |
| | carboxamide |
| 017 | (S)-N-(2-(8-methylimidazo[1,5-a]pyridin-3-yl)propan-2-yl)-1-(2,2,2- |
| | trifluoroethyl)piperazine-2-carboxamide |
| 018 | (S)-1-(2-fluoro-2-methylpropyl)-N-(2-(8-methylimidazo[1,5-a]pyridin-3-yl)propan- |
| | 2-yl)piperazine-2-carboxamide |
| 019 | (S)-1-(2,2-difluoropropyl)-N-(2-(8-methylimidazo[1,5-a]pyridin-3-yl)propan-2- |
| | yl)piperazine-2-carboxamide |
| 020 | (S)-N-(2-(8-methylimidazo[1,5-a]pyridin-3-yl)propan-2-yl)-1-((1- |
| | (trifluoromethyl)cyclopropyl)methyl)piperazine-2-carboxamide |
| 021 | (S)-1-(3,3-difluorocyclobutyl)-N-(2-(8-methylimidazo[1,5-a]pyridin-3-yl)propan-2- |
| | yl)piperazine-2-carboxamide |
| 022 | (S)-1-((2-cyclopropylpyridin-4-yl)methyl)-N-(2-(8-methylimidazo[1,5-a]pyridin-3- |
| | yl)propan-2-yl)piperazine-2-carboxamide |
| 023 | (S)-1-(2-hydroxy-2-methylpropyl)-N-(2-(8-methylimidazo[1,5-a]pyridin-3-yl)propan- |
| | 2-yl)piperazine-2-carboxamide |
| 024 | (S)-1-((4,4-difluoro-1-methylcyclohexyl)methyl)-N-(2-(8-methylimidazo[1,5-a]pyridin- |
| | 3-yl)propan-2-yl)piperazine-2-carboxamide |
| 025 | (S)-N-(2-(8-methylimidazo[1,5-a]pyridin-3-yl)propan-2-yl)-1-(3- |
| | (trifluoromethyl)benzyl)piperazine-2-carboxamide |
| 026 | (S)-1-(2-fluorobenzyl)-N-(2-(8-methylimidazo[1,5-a]pyridin-3-yl)propan-2-yl)piperazine- |
| | 2-carboxamide |
| 027 | (S)-1-(4-fluorobenzyl)-N-(2-(8-methylimidazo[1,5-a]pyridin-3-yl)propan-2-yl)piperazine- |
| | 2-carboxamide |
| 028 | (S)-N-(2-(8-methylimidazo[1,5-a]pyridin-3-yl)propan-2-yl)-1-(4- |
| | (trifluoromethyl)benzyl)piperazine-2-carboxamide |
| 029 | (S)-1-(3-fluorobenzyl)-N-(2-(8-methylimidazo[1,5-a]pyridin-3-yl)propan-2-yl)piperazine- |
| | 2-carboxamide |
| 030 | (S)-N-(2-(8-methylimidazo[1,5-a]pyridin-3-yl)propan-2-yl)-1-((6-(trifluoromethyl)pyridin- |
| | 3-yl)methyl)piperazine-2-carboxamide |
| 031 | (S)-N-(2-(8-methylimidazo[1,5-a]pyridin-3-yl)propan-2-yl)-1-((3-methyloxetan-3- |
| | yl)methyl)piperazine-2-carboxamide |
| 032 | (S)-1-(4,4-difluorocyclohexyl)-N-(2-(8-methylimidazo[1,5-a]pyridin-3-yl)propan-2- |
| | yl)piperazine-2-carboxamide |
| 033 | (S)-1-(cyclobutylmethyl)-N-(2-(8-methylimidazo[1,5-a]pyridin-3-yl)propan-2-yl)piperazine- |
| | 2-carboxamide |
| 034 | (S)-1-isobutyl-N-(2-(8-methylimidazo[1,5-a]pyridin-3-yl)propan-2-yl)piperazine-2- |
| | carboxamide |
| 035 | (S)-1-isopropyl-N-(2-(8-methylimidazo[1,5-a]pyridin-3-yl)propan-2-yl)piperazine-2- |
| | carboxamide |
| 036 | (S)-N-(2-(8-methylimidazo[1,5-a]pyridin-3-yl)propan-2-yl)-1-((2-(trifluoromethyl)pyridin-4- |
| | yl)methyl)piperazine-2-carboxamide |
| 037 | (S)-N-(2-(8-methylimidazo[1,5-a]pyridin-3-yl)propan-2-yl)-1-((4-methyltetrahydro-2H- |
| | pyran-4-yl)methyl)piperazine-2-carboxamide |
| 038 | (S)-N-(2-(8-methylimidazo[1,5-a]pyridin-3-yl)propan-2-yl)-1-((2-(trifluoromethyl)pyrimidin- |
| | 5-yl)methyl)piperazine-2-carboxamide |
| 039 | (S)-1-(3-methoxy-2,2-dimethylpropyl)-N-(2-(8-methylimidazo[1,5-a]pyridin-3-yl)propan-2- |
| | yl)piperazine-2-carboxamide |
| 040 | (S)-1-isopropyl-N-(2-(7-methyl-1H-indazol-3-yl)propan-2-yl)piperazine-2-carboxamide |
| 041 | (S)-1-ethyl-N-(2-(7-methyl-1H-indazol-3-yl)propan-2-yl)piperazine-2-carboxamide |
| 042 | (S)-N-(2-(8-methylimidazo[1,5-a]pyridin-3-yl)propan-2-yl)-1-(oxetan-3-ylmethyl)piperazine- |
| | 2-carboxamide |
| 043 | (S)-4-methyl-N-(2-(8-methylimidazo[1,5-a]pyridin-3-yl)propan-2-yl)piperazine-2- |
| | carboxamide |
| 044 | (S)-4-methyl-1-((1-methyl-1H-pyrazol-4-yl)methyl)-N-(2-(8-methylimidazo[1,5-a]pyridin-3- |
| | yl)propan-2-yl)piperazine-2-carboxamide |
| 045 | (S)-4-methyl-N-(2-(8-methylimidazo[1,5-a]pyridin-3-yl)propan-2-yl)-1-(2,2,2- |
| | trifluoroethyl)piperazine-2-carboxamide |
| 046 | (S)-1,4-dimethyl-N-(2-(8-methylimidazo[1,5-a]pyridin-3-yl)propan-2-yl)piperazine-2- |
| | carboxamide |
| 047 | (S)-4-methyl-N-(2-(8-methylimidazo[1,5-a]pyridin-3-yl)propan-2-yl)-1-((tetrahydro-2H- |
| | pyran-4-yl)methyl)piperazine-2-carboxamide |
| 048 | (S)-4-methyl-N-(2-(8-methylimidazo[1,5-a]pyridin-3-yl)propan-2-yl)-1-(oxetan-3- |
| | ylmethyl)piperazine-2-carboxamide |
| 049 | (S)-N-(2-(8-methylimidazo[1,5-a]pyridin-3-yl)propan-2-yl)-4-(2,2,2- |
| | trifluoroethyl)piperazine-2-carboxamide |
| 050 | (S)-4-(2,2-difluoroethyl)-N-(2-(8-methylimidazo[1,5-a]pyridin-3-yl)propan-2-yl)piperazine- |
| | 2-carboxamide |
| 051 | (S)-4-(2-methoxyethyl)-N-(2-(8-methylimidazo[1,5-a]pyridin-3-yl)propan-2-yl)piperazine-2- |
| | carboxamide |
| 052 | (S)-4-isopropyl-N-(2-(8-methylimidazo[1,5-a]pyridin-3-yl)propan-2-yl)piperazine-2- |
| | carboxamide |
| 053 | (S)-4-cyclobutyl-N-(2-(8-methylimidazo[1,5-a]pyridin-3-yl)propan-2-yl)piperazine-2- |
| | carboxamide |
| 054 | (S)-4-ethyl-N-(2-(8-methylimidazo[1,5-a]pyridin-3-yl)propan-2-yl)piperazine-2-carboxamide |
| 055 | (S)-4-(4,4-difluorocyclohexyl)-N-(2-(8-methylimidazo[1,5-a]pyridin-3-yl)propan-2- |
| | yl)piperazine-2-carboxamide |
| 056 | (S)-N-(2-(8-methylimidazo[1,5-a]pyridin-3-yl)propan-2-yl)-4-(tetrahydro-2H-pyran-4- |
| | yl)piperazine-2-carboxamide |
| 057 | (S)-N-(2-(8-methylimidazo[1,5-a]pyridin-3-yl)propan-2-yl)-4-((tetrahydro-2H-pyran-4- |
| | yl)methyl)piperazine-2-carboxamide |
| 058 | (S)-4-benzyl-N-(2-(8-methylimidazo[1,5-a]pyridin-3-yl)propan-2-yl)piperazine-2- |
| | carboxamide |
| 059 | (S)-N-(2-(8-methylimidazo[1,5-a]pyridin-3-yl)propan-2-yl)-4-(pyridin-4-yl- |
| | methyl)piperazine-2-carboxamide |
| 060 | (S)-N-(2-(8-methylimidazo[1,5-a]pyridin-3-yl)propan-2-yl)-4-(pyridin-2-yl- |
| | methyl)piperazine-2-carboxamide |
| 061 | (S)-N-(2-(8-methylimidazo[1,5-a]pyridin-3-yl)propan-2-yl)-4-(pyridin-3-yl- |
| | methyl)piperazine-2-carboxamide |
| 062 | (S)-N-(2-(8-methylimidazo[1,5-a]pyridin-3-yl)propan-2-yl)-4-(oxazol-5-ylmethyl)piperazine- |
| | 2-carboxamide |
| 063 | (S)-N-(2-(8-methylimidazo[1,5-a]pyridin-3-yl)propan-2-yl)-4-(oxazol-4-ylmethyl)piperazine- |
| | 2-carboxamide |
| 064 | (S)-4-((1-methyl-3-(trifluoromethyl)-1H-pyrazol-5-yl)methyl)-N-(2-(8-methylimidazo[1,5- |
| | a]pyridin-3-yl)propan-2-yl)piperazine-2-carboxamide |
| 065 | (S)-N-(2-(8-methylimidazo[1,5-a]pyridin-3-yl)propan-2-yl)-4-(oxazol-2-ylmethyl)piperazine- |
| | 2-carboxamide |
| 066 | (S)-4-((1-methyl-1H-pyrazol-3-yl)methyl)-N-(2-(8-methylimidazo[1,5-a]pyridin-3-yl)propan- |
| | 2-yl)piperazine-2-carboxamide |
| 067 | (S)-4-((4,4-difluorocyclohexyl)methyl)-N-(2-(8-methylimidazo[1,5-a]pyridin-3-yl)propan-2- |
| | yl)piperazine-2-carboxamide |
| 068 | (S)-4-(cyclobutylmethyl)-N-(2-(8-methylimidazo[1,5-a]pyridin-3-yl)propan-2-yl)piperazine- |
| | 2-carboxamide |
| 069 | (S)-4-isobutyl-N-(2-(8-methylimidazo[1,5-a]pyridin-3-yl)propan-2-yl)piperazine-2- |
| | carboxamide |
| 070 | (S)-N-(2-(8-methylimidazo[1,5-a]pyridin-3-yl)propan-2-yl)-4-propylpiperazine-2- |
| | carboxamide |
| 071 | (S)-4-((1-methyl-1H-pyrazol-4-yl)methyl)-N-(2-(8-methylimidazo[1,5-a]pyridin-3-yl)propan- |
| | 2-yl)piperazine-2-carboxamide |
| 072 | (S)-4-((1-methyl-1H-pyrazol-5-yl)methyl)-N-(2-(8-methylimidazo[1,5-a]pyridin-3-yl)propan- |
| | 2-yl)piperazine-2-carboxamide |
| 073 | (S)-4-((1-isopropyl-1H-pyrazol-3-yl)methyl)-N-(2-(8-methylimidazo[1,5-a]pyridin-3- |
| | yl)propan-2-yl)piperazine-2-carboxamide |
| 074 | (S)-4-((1-isopropyl-1H-pyrazol-4-yl)methyl)-N-(2-(8-methylimidazo[1,5-a]pyridin-3- |
| | yl)propan-2-yl)piperazine-2-carboxamide |
| 075 | (S)-N-(2-(8-methylimidazo[1,5-a]pyridin-3-yl)propan-2-yl)-4-(oxetan-3-yl)piperazine-2- |
| | carboxamide |
| 076 | (S)-N-(2-(8-methylimidazo[1,5-a]pyridin-3-yl)propan-2-yl)-4-(oxetan-3-ylmethyl)piperazine- |
| | 2-carboxamide |
| 077 | (S)-4-(cyclopropanecarbonyl)-N-(2-(8-methylimidazo[1,5-a]pyridin-3-yl)propan-2- |
| | yl)piperazine-2-carboxamide |
| 078 | (S)-N-(2-(8-methylimidazo[1,5-a]pyridin-3-yl)propan-2-yl)-4-(tetrahydro-2H-pyran-4- |
| | carbonyl)piperazine-2-carboxamide |
| 079 | (S)-N-(2-(8-methylimidazo[1,5-a]pyridin-3-yl)propan-2-yl)-4-(2-(tetrahydro-2H-pyran-4- |
| | yl)acetyl)piperazine-2-carboxamide |
| 080 | (S)-4-(3,3-difluorocyclobutane-1-carbonyl)-N-(2-(8-methylimidazo[1,5-a]pyridin-3- |
| | yl)propan-2-yl)piperazine-2-carboxamide |
| 081 | (S)-4-(1-isopropyl-1H-pyrazole-4-carbonyl)-N-(2-(8-methylimidazo[1,5-a]pyridin-3- |
| | yl)propan-2-yl)piperazine-2-carboxamide |
| 082 | (S)-4-(1-methyl-1H-pyrazole-5-carbonyl)-N-(2-(8-methylimidazo[1,5-a]pyridin-3-yl)propan- |
| | 2-yl)piperazine-2-carboxamide |
| 083 | (S)-4-(1-methyl-1H-pyrazole-4-carbonyl)-N-(2-(8-methylimidazo[1,5-a]pyridin-3-yl)propan- |
| | 2-yl)piperazine-2-carboxamide |
| 084 | (S)-4-(1-methyl-1H-pyrazole-3-carbonyl)-N-(2-(8-methylimidazo[1,5-a]pyridin-3-yl)propan- |
| | 2-yl)piperazine-2-carboxamide |
| 085 | (S)-1-benzyl-N-(2-(8-methylimidazo[1,5-a]pyridin-3-yl)propan-2-yl)piperazine-2- |
| | carboxamide |
| 086 | (S)-N-(2-(8-methylimidazo[1,5-a]pyridin-3-yl)propan-2-yl)-1-((tetrahydro-2H-pyran-4- |
| | yl)methyl)piperazine-2-carboxamide |
| 087 | (S)-1-((4,4-difluorocyclohexyl)methyl)-N-(2-(8-methylimidazo[1,5-a]pyridin-3-yl)propan-2- |
| | yl)piperazine-2-carboxamide |
| 088 | (S)-N-(2-(8-methylimidazo[1,5-a]pyridin-3-yl)propan-2-yl)-1-((6-methylpyridin-3- |
| | yl)methyl)piperazine-2-carboxamide |
| 089 | (S)-1-((3,3-difluorocyclobutyl)methyl)-N-(2-(8-methylimidazo[1,5-a]pyridin-3-yl)propan-2- |
| | yl)piperazine-2-carboxamide |
| 090 | (S)-1-((1-methyl-1H-pyrazol-4-yl)methyl)-N-(2-(8-methylimidazo[1,5-a]pyridin-3-yl)propan- |
| | 2-yl)piperazine-2-carboxamide |
| 091 | (S)-N-(2-(8-methylimidazo[1,5-a]pyridin-3-yl)propan-2-yl)-1-((2-methylpyridin-4- |
| | yl)methyl)piperazine-2-carboxamide |
| 092 | (S)-1-(2-methoxyethyl)-N-(2-(8-methylimidazo[1,5-a]pyridin-3-yl)propan-2-yl)piperazine-2- |
| | carboxamide |
| 093 | rac-N-(2-(8-methylimidazo[1,5-a]pyridin-3-yl)propan-2-yl)-1-phenylpiperazine-2-carboxamide |
| 094 | (S)-1-methyl-N-(2-(7-methyl-1H-indazol-3-yl)propan-2-yl)piperazine-2-carboxamide |
| 095 | (S)-4-methyl-N-(2-(7-methyl-1H-indazol-3-yl)propan-2-yl)piperazine-2-carboxamide |
| 096 | (S)-N-(2-(7-methyl-1H-indazol-3-yl)propan-2-yl)-1-((tetrahydro-2H-pyran-4- |
| | yl)methyl)piperazine-2-carboxamide |
| 097 | (S)-1-(2-methoxyethyl)-N-(2-(7-methyl-1H-indazol-3-yl)propan-2-yl)piperazine-2- |
| | carboxamide |
| 098 | (S)-N-(2-(7-methyl-1H-indazol-3-yl)propan-2-yl)-1-(2,2,2-trifluoroethyl)piperazine-2- |
| | carboxamide |
| 099 | (S)-4-methyl-N-(2-(1-methyl-1H-indazol-3-yl)propan-2-yl)piperazine-2-carboxamide |
| 100 | (S)-1-methyl-N-(2-(1-methyl-1H-indazol-3-yl)propan-2-yl)piperazine-2-carboxamide |
| 101 | (S)-1-(2-methoxyethyl)-N-(2-(1-methyl-1H-indazol-3-yl)propan-2-yl)piperazine-2- |
| | carboxamide |
| 102 | (S)-N-(2-(1-methyl-1H-indazol-3-yl)propan-2-yl)-1-(2,2,2-trifluoroethyl)piperazine-2- |
| | carboxamide |
| 103 | (S)-N-(2-(1-methyl-1H-indazol-3-yl)propan-2-yl)-1-((tetrahydro-2H-pyran-4- |
| | yl)methyl)piperazine-2-carboxamide |
| 104 | (S)-N-(2-(8-chloroimidazo[1,5-a]pyridin-3-yl)propan-2-yl)-1-methylpiperazine-2-carboxamide |
| 105 | (S)-N-(2-(8-chloroimidazo[1,5-a]pyridin-3-yl)propan-2-yl)-4-methylpiperazine-2-carboxamide |
| 106 | (S)-N-(2-(8-chloroimidazo[1,5-a]pyridin-3-yl)propan-2-yl)-1-((1-methyl-1H-pyrazol-4- |
| | yl)methyl)piperazine-2-carboxamide |
| 107 | (S)-N-(2-(8-chloroimidazo[1,5-a]pyridin-3-yl)propan-2-yl)-1-((3,3- |
| | difluorocyclobutyl)methyl)piperazine-2-carboxamide |
| 108 | (S)-N-(2-(8-chloroimidazo[1,5-a]pyridin-3-yl)propan-2-yl)piperazine-2-carboxamide |
| 109 | (R)-N-(2-(8-chloroimidazo[1,5-a]pyridin-3-yl)propan-2-yl)-1-methylpiperazine-2-carboxamide |
| 110 | (S)-1-methyl-N-(2-(8-(trifluoromethyl)imidazo[1,5-a]pyridin-3-yl)propan-2-yl)piperazine-2- |
| | carboxamide |
| 111 | (S)-1-((1-methyl-1H-pyrazol-4-yl)methyl)-N-(2-(8-(trifluoromethyl)imidazo[1,5-a]pyridin-3- |
| | yl)propan-2-yl)piperazine-2-carboxamide |
| 112 | (S)-4-methyl-N-(2-(8-(trifluoromethyl)imidazo[1,5-a]pyridin-3-yl)propan-2-yl)piperazine-2- |
| | carboxamide |
| 113 | (S)-1-((3,3-difluorocyclobutyl)methyl)-N-(2-(8-(trifluoromethyl)imidazo[1,5-a]pyridin-3- |
| | yl)propan-2-yl)piperazine-2-carboxamide |
| 114 | (S)-N-(2-(1,8-dimethylimidazo[1,5-a]pyridin-3-yl)propan-2-yl)-1-methylpiperazine-2- |
| | carboxamide |
| 115 | (S)-1-((3,3-difluorocyclobutyl)methyl)-N-(2-(1,8-dimethylimidazo[1,5-a]pyridin-3-yl)propan- |
| | 2-yl)piperazine-2-carboxamide |
| 116 | (S)-N-(2-(1,8-dimethylimidazo[1,5-a]pyridin-3-yl)propan-2-yl)-4-methylpiperazine-2- |
| | carboxamide |
| 117 | (S)-N-(2-(1,8-dimethylimidazo[1,5-a]pyridin-3-yl)propan-2-yl)-1-((1-methyl-1H-pyrazol-4- |
| | yl)methyl)piperazine-2-carboxamide |
| 118 | (S)-N-(2-(8-cyclopropylimidazo[1,5-a]pyridin-3-yl)propan-2-yl)-1-methylpiperazine-2- |
| | carboxamide |
| 119 | (S)-N-(2-(8-cyclopropylimidazo[1,5-a]pyridin-3-yl)propan-2-yl)-4-methylpiperazine-2- |
| | carboxamide |
| 120 | (S)-N-(2-(8-cyclopropylimidazo[1,5-a]pyridin-3-yl)propan-2-yl)-1-((1-methyl-1H-pyrazol-4- |
| | yl)methyl)piperazine-2-carboxamide |
| 121 | (S)-N-(2-(8-cyclopropylimidazo[1,5-a]pyridin-3-yl)propan-2-yl)-1-((3,3- |
| | difluorocyclobutyl)methyl)piperazine-2-carboxamide |
| 122 | (S)-N-(2-(7-methyl-1H-indazol-3-yl)propan-2-yl)-1-(oxetan-3-ylmethyl)piperazine-2- |
| | carboxamide |
| 123 | (S)-N-(2-(1-methyl-1H-indazol-3-yl)propan-2-yl)-1-(oxetan-3-ylmethyl)piperazine-2- |
| | carboxamide |
| 124 | (S)-1-methyl-N-(2-(7-methylbenzo[d]isoxazol-3-yl)propan-2-yl)piperazine-2-carboxamide |
| 125 | (S)-N-(2-(8-chloro-[1,2,4]triazolo[4,3-a]pyridin-3-yl)propan-2-yl)-1-methylpiperazine- |
| | 2-carboxamide |
| 126 | (S)-N-(2-(1,7-dimethyl-1H-indazol-3-yl)propan-2-yl)-1-methylpiperazine-2-carboxamide |
| 127 | (S)-1-(2,2-difluoroethyl)-N-(2-(8-methylimidazo[1,5-a]pyridin-3-yl)propan-2-yl)piperazine- |
| | 2-carboxamide |
| 128 | (S)-N-(2-(8-methoxyimidazo[1,5-a]pyridin-3-yl)propan-2-yl)-1-methylpiperazine-2- |
| | carboxamide |
| 129 | (S)-N-(2-(7-fluoro-1H-indazol-3-yl)propan-2-yl)-1-methylpiperazine-2-carboxamide |

or a physiologically acceptable salt thereof.

Another aspect of the invention relates to a medicament comprising a compound according to the invention as described above.

Another aspect of the invention relates to a pharmaceutical dosage form comprising a compound according to the invention. Preferably, the pharmaceutical dosage form comprises a compound according to the invention and one or more pharmaceutical excipients such as physiologically acceptable carriers, additives and/or auxiliary substances; and optionally one or more further pharmacologically active ingredient. Examples of suitable physiologically acceptable carriers, additives and/or auxiliary substances are fillers, solvents, diluents, colorings and/or binders. These substances are known to the person skilled in the art (see H. P. Fiedler, Lexikon der Hilfsstoffe fur Pharmazie, Kosmetik und angrenzende Gebiete, Editio Cantor Aulendoff).

The pharmaceutical dosage form according to the invention is preferably for systemic, topical or local administration, preferably for oral administration. Therefore, the pharmaceutical dosage form can be in form of a liquid, semisolid or solid, e.g. in the form of injection solutions, drops, juices, syrups, sprays, suspensions, tablets, patches, films, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and can also be administered as such.

The pharmaceutical dosage form according to the invention is preferably prepared with the aid of conventional means, devices, methods and processes known in the art. The amount of the compound according to the invention to be administered to the patient may vary and is e.g. dependent on the patient's weight or age and also on the type of administration, the indication and the severity of the disorder. Preferably 0.001 to 100 mg/kg, more preferably 0.05 to 75 mg/kg, most preferably 0.05 to 50 mg of a compound according to the invention are administered per kg of the patient's body weight.

Therefore, another aspect of the invention relates to the pharmaceutical dosage form according to the invention for use in the treatment of pain. Still another aspect of the invention relates to a method of treatment of pain; comprising the administration of a pharmaceutical dosage form according to the invention to a subject in need thereof, preferably a human.

### Methods of Preparation:

Another aspect of the invention relates to a process for the preparation of the compounds according to the invention. Suitable processes for the synthesis of the compounds according to the invention are known in principle to the person skilled in the art.

Preferred synthesis routes are described below:

The compounds according to the invention can be obtained via different synthesis routes. Depending on the synthesis route, different intermediates are prepared and subsequently further reacted. For all amines and carboxylic acids described below it is appreciated that their corresponding salts can be reacted similarly under the appropriate conditions. W refers to a heterobicyclic aromatic structure according to the claims of the invention (see Figure 1).

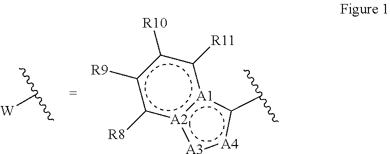

According to a first process, compounds of Formula (I) may be prepared from compounds of Formulae (VI), (V), (IV) or (II), as illustrated by Scheme 1.

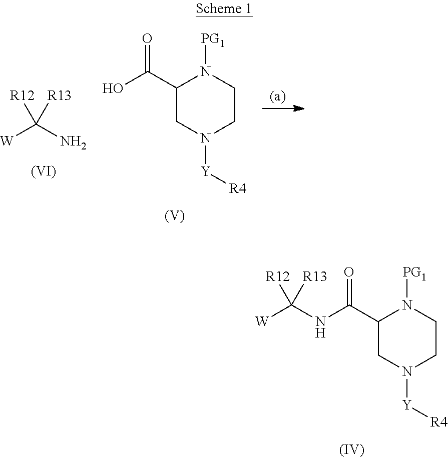

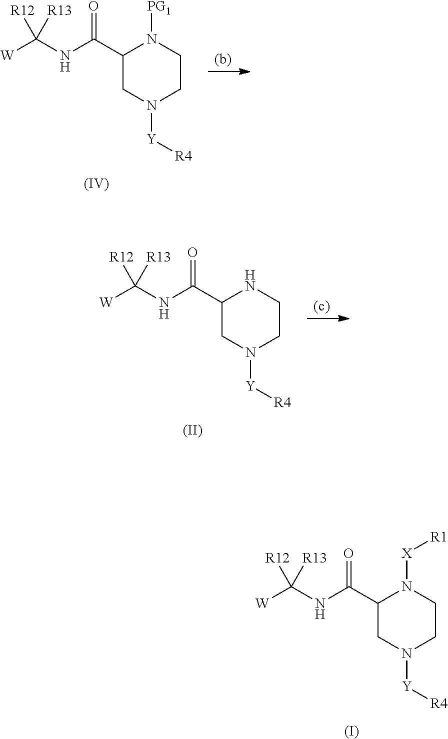

W refers to a bicycloaryl as defined above. PG₁ is a suitable protecting group, preferably Boc, Cbz or Fmoc. X, Y, R1 R4, R12 and R13 refer to residues as defined above.

The compounds of Formulae (VI) and (V) are reacted in an amide bond forming reaction (a) to yield compounds of Formula (IV). Typical reaction conditions comprise, reaction of the amine of Formula (VI) with a carboxylic acid of Formula (V) in the presence of a suitable coupling reagent and a suitable base in a suitable solvent at an appropriate temperature. The compounds of Formula (IV) are then deprotected in a reaction (b) to yield compounds of Formula (II). Typical reaction conditions comprise, reaction of the compound of Formula (IV) with a suitable reagent in a suitable solvent at an appropriate temperature. Compounds of Formula (IV) are reacted in an alkylation or reductive amination reaction (c) to yield compounds of Formula (I). Typical reaction conditions comprise, reaction of the compound of Formula (II) with a suitable alkylating reagent in the presence of a suitable base in a suitable solvent at an appropriate temperature or reaction of of the compound of Formula (II) with a suitable carbonyl compound in the presence of a suitable reducing agent and a suitable catalyst in a suitable solvent at an appropriate temperature. Preparations according to above scheme are exemplified in the preparations described below for examples SC-044, SC-045 and SC-047.

According to a second process, compounds of Formula (II) may be prepared from compounds of Formulae (VI), (IX), (VIII), (VII) or (IV) as illustrated by Scheme 2.

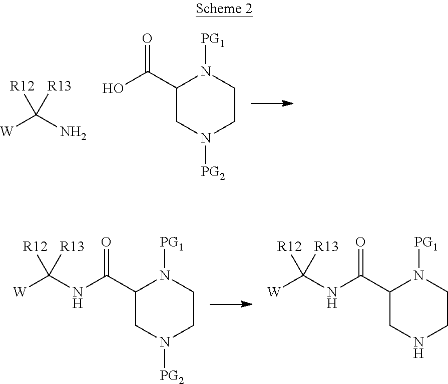

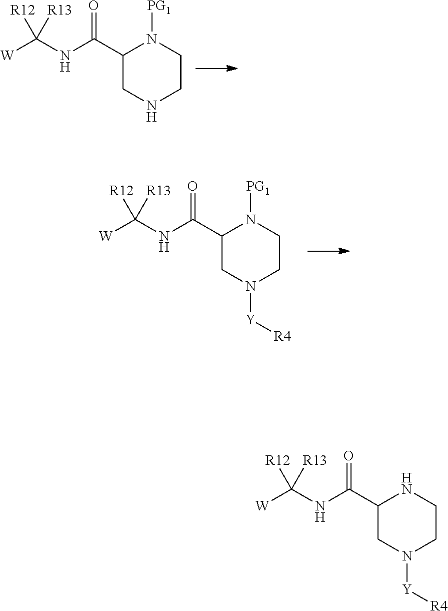

W refers to a bicycloaryl as defined above. PG₁ and PG₂ are suitable disparate protecting groups, preferably Boc, Cbz or Fmoc. Y, R4, R12 and R13 refers to a residue as defined above.

The compounds of Formulae (VI) and (IX) are reacted in an amide bond forming reaction (a) to yield compounds of Formula (VIII). Typical reaction conditions comprise, reaction of the amine of Formula (VI) with a carboxylic acid of Formula (IX) in the presence of a suitable coupling reagent and a suitable base in a suitable solvent at an appropriate temperature. The compounds of Formula (VIII) are then selectively deprotected in a reaction (b) to yield compounds of Formula (VII). Typical reaction conditions comprise, reaction of the compound of Formula (VIII) with a suitable reagent in a suitable solvent at an appropriate temperature. Compounds of Formula (VII) are reacted in an alkylation or reductive amination or acylation reaction (c) to yield compounds of Formula (IV). Typical reaction conditions comprise, reaction of the compound of Formula (VII) with a suitable alkylating reagent in the presence of a suitable base in a suitable solvent at an appropriate temperature or reaction of the compound of Formula (VII) with a suitable carbonyl compound in the presence of a suitable reducing agent and a suitable catalyst in a suitable solvent at an appropriate temperature or reaction of the compound of Formula (VII) with a suitable carboxylic acid in the presence of a suitable coupling reagent and a suitable base in a suitable solvent at an appropriate temperature. The compounds of Formula (IV) are then deprotected in a reaction (d) to yield compounds of Formula (II). Preparations according to above scheme are exemplified in the preparations described below for examples SC-050, SC-052 and SC-077.

According to a third process, compounds of Formula (II) may be prepared from compounds of Formulae (XI), (X), (V), (VI), (IV), (XIII) or (XII) as illustrated by Scheme 3.

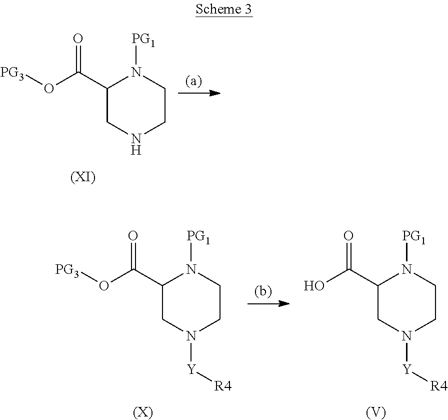

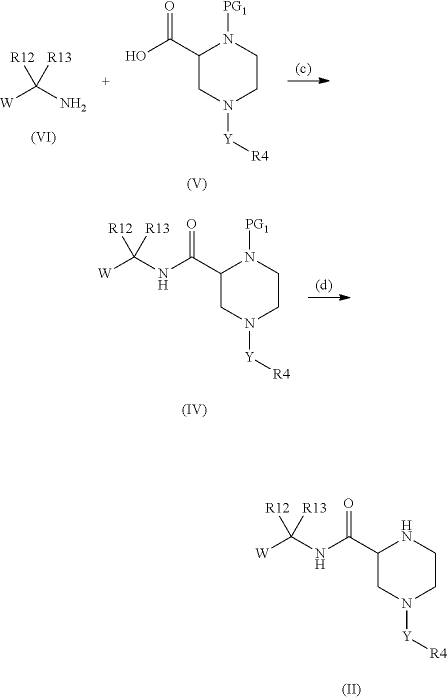

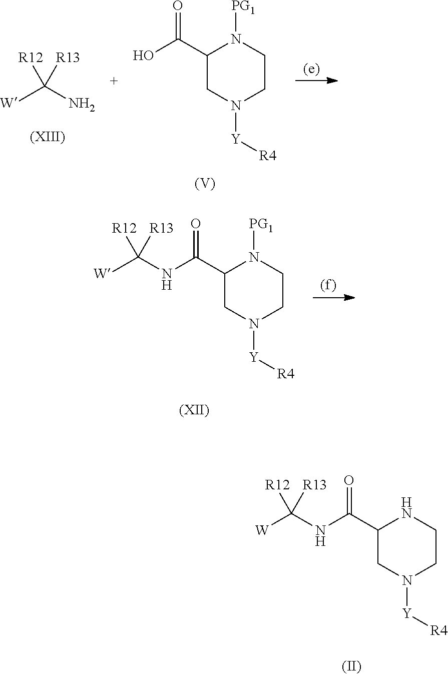

W refers to a bicycloaryl as defined above. W′ refers to a suitably protected form of a bicycloaryl as defined above. PG₁ is a suitable protecting group, preferably Boc, Cbz or Fmoc. PG₃ is a suitable protecting group, preferably Me. Y, R4, R12 and R13 refers to a residue as defined above.

Compounds of Formula (XI) are reacted in an alkylation or reductive amination reaction (a) to yield compounds of Formula (X). Typical reaction conditions comprise, reaction of the compound of Formula (XI) with a suitable alkylating reagent in the presence of a suitable base in a suitable solvent at an appropriate temperature or reaction of the compound of Formula (XI) with a suitable carbonyl compound in the presence of a suitable reducing agent and a suitable catalyst in a suitable solvent at an appropriate temperature. The compounds of Formula (X) are deprotected in a reaction (b) to yield compounds of Formula (V). Typical reaction conditions comprise, reaction of the compound of Formula (X) with a suitable reagent in a suitable solvent at an appropriate temperature. The compounds of Formulae (VI) and (V) are reacted in an amide bond forming reaction (c) to yield compounds of Formula (IV). Typical reaction conditions comprise, reaction of the amine of Formula (VI) with a carboxylic acid of Formula (V) in the presence of a suitable coupling reagent and a suitable base in a suitable solvent at an appropriate temperature. The compounds of Formula (IV) are deprotected in a reaction (d) to yield compounds of Formula (II). Typical reaction conditions comprise, reaction of the compound of Formula (IV) with a suitable reagent in a suitable solvent at an appropriate temperature. Similarly, compounds of Formulae (XIII) and (V) are reacted in an amide bond forming reaction (e) to yield compounds of Formula (XII) and compounds of Formula (XII) are deprotected in a reaction (f) to yield compounds of Formula (II) with concomitant deprotection of W′. Preparations according to above scheme are exemplified in the preparations described below for examples SC-095, SC-099 and SC-105.

According to a fourth process, compounds of Formula (III) may be prepared from compounds of Formulae (VI), (IX), (VIII), (XV), (XIV), (XIII), (IX), (XVIII), (XVII) or (XVI) as illustrated by Scheme 4.

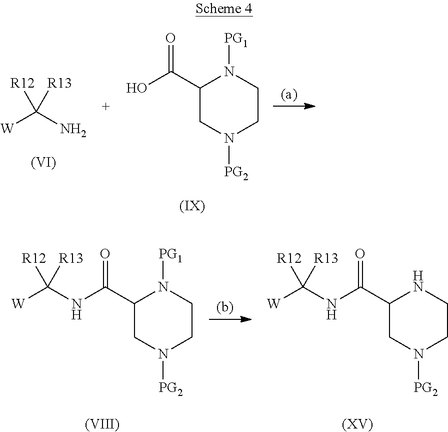

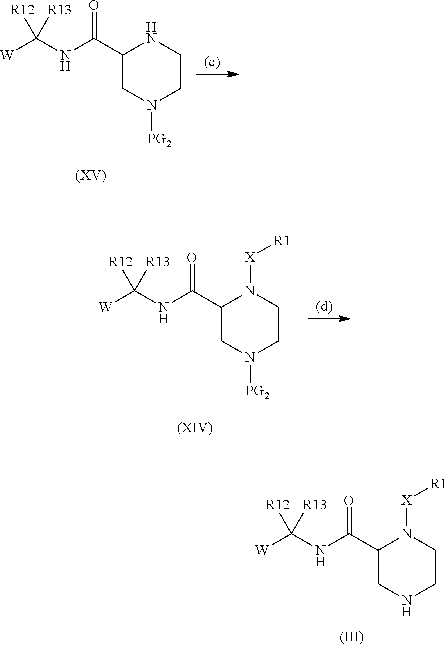

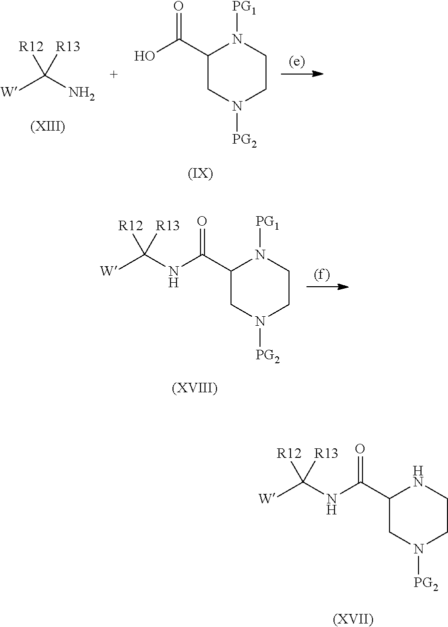

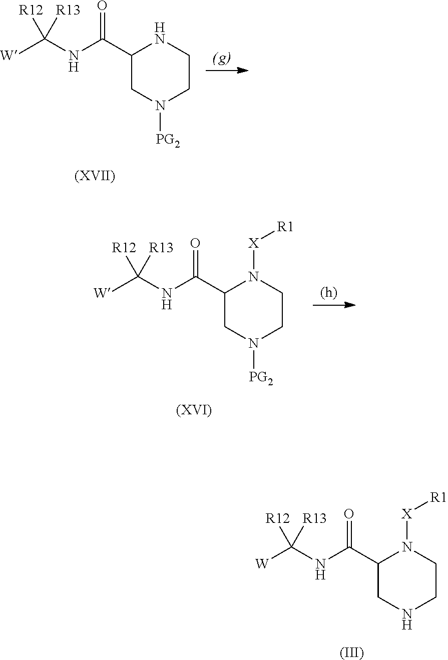

W refers to a bicycloaryl as defined above. W′ refers to a suitably protected form of a bicycloaryl as defined above. PG₁ and PG₂ are suitable disparate protecting groups, preferably Boc, Cbz or Fmoc. X, R1, R12 and R13 refers to a residue as defined above.

The compounds of Formulae (VI) and (IX) are reacted in an amide bond forming reaction (a) to yield compounds of Formula (VIII). Typical reaction conditions comprise, reaction of the amine of Formula (VI) with a carboxylic acid of Formula (IX) in the presence of a suitable coupling reagent and a suitable base in a suitable solvent at an appropriate temperature. The compounds of Formula (VIII) are selectively deprotected in a reaction (b) to yield compounds of Formula (XV). Typical reaction conditions comprise, reaction of the compound of Formula (VIII) with a suitable reagent in a suitable solvent at an appropriate temperature. Compounds of Formula (XV) are reacted in an alkylation or reductive amination or acylation reaction (c) to yield compounds of Formula (XIV). Typical reaction conditions comprise, reaction of the compound of Formula (XV) with a suitable alkylating reagent in the presence of a suitable base in a suitable solvent at an appropriate temperature or reaction of the compound of Formula (XV) with a suitable carbonyl compound in the presence of a suitable reducing agent and a suitable catalyst in a suitable solvent at an appropriate temperature or reaction of the compound of Formula (XV) with a suitable carboxylic acid in the presence of a suitable coupling reagent and a suitable base in a suitable solvent at an appropriate temperature. The compounds of Formula (XIV) are deprotected in a reaction (d) to yield compounds of Formula (III). Typical reaction conditions comprise, reaction of the compound of Formula (XIV) with a suitable reagent in a suitable solvent at an appropriate temperature. Similarly, the above process applies to intermediates (XIII), (XVIII), (XVII) and (XVI) in which W is suitably protected throughout steps (e), (f) and (g) and where reaction (h) leads to concomitant deprotection of W′. Preparations according to above scheme are exemplified in the preparations described below for examples SC-010, SC-017 and SC-040.

According to a fifth process, compounds of Formula (III) may be prepared from compounds of Formulae (XXIII), (XIX), (XXII), (XXI), (VI), (XIV), (XIII), (XVI) or (XX) as illustrated by Scheme 5.

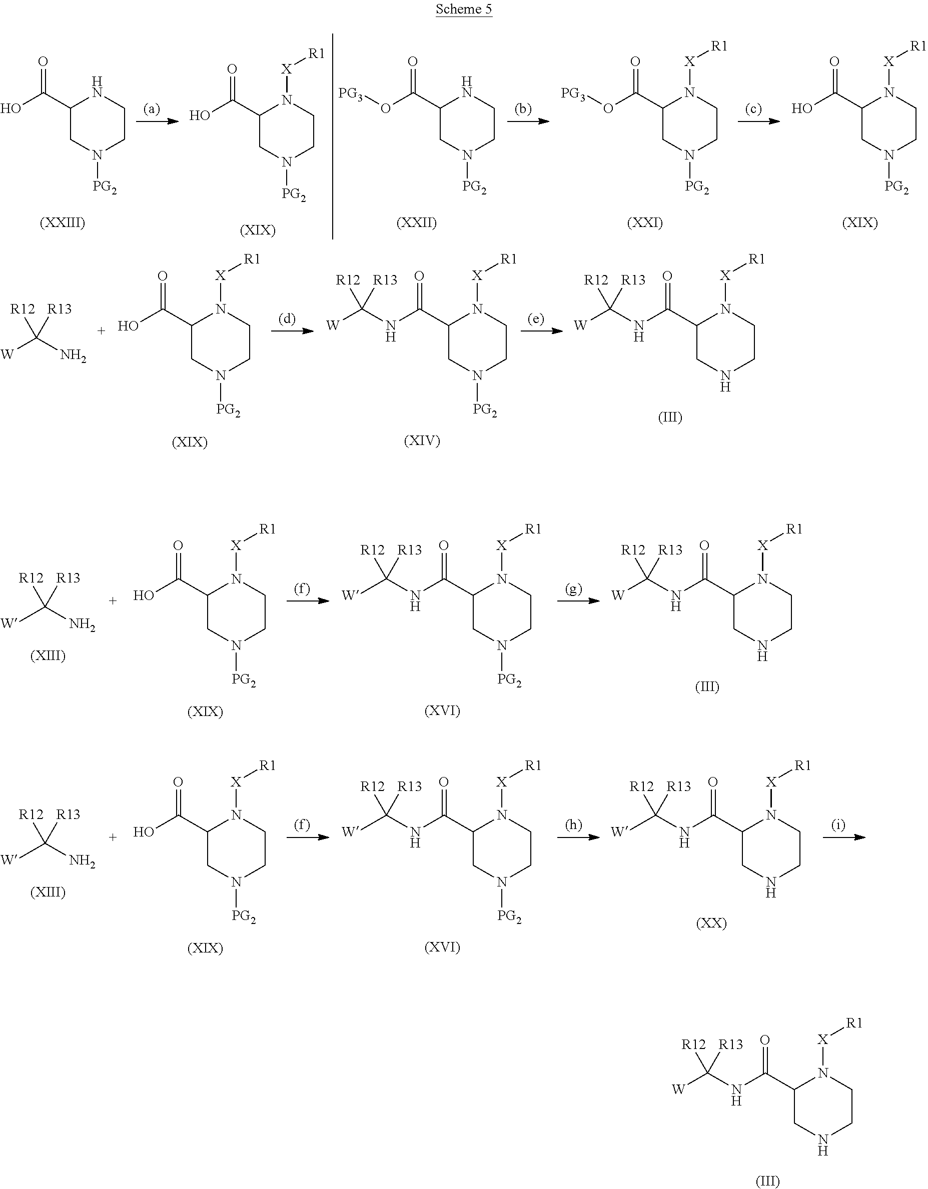

W refers to a bicycloaryl as defined above. W′ refers to a suitably protected form of a bicycloaryl as defined above. PG₂ is a suitable protecting group, preferably Boc, Cbz or Fmoc. PG₃ is a suitable protecting group, preferably Me. X, R1, R12 and R13 refers to a residue as defined above.

Compounds of Formula (XXIII) are reacted in a reductive amination reaction (a) to yield compounds of Formula (XIX). Typical reaction conditions comprise, reaction of the compound of Formula (XXIII) with a suitable carbonyl compound in the presence of a suitable reducing agent and a suitable catalyst in a suitable solvent at an appropriate temperature. Compounds of Formula (XXII) are reacted in an alkylation or reductive amination reaction (b) to yield compounds of Formula (XXI). Typical reaction conditions comprise, reaction of the compound of Formula (XXII) with a suitable alkylating reagent in the presence of a suitable base in a suitable solvent at an appropriate temperature or reaction of the compound of Formula (XXII) with a suitable carbonyl compound in the presence of a suitable reducing agent and a suitable catalyst in a suitable solvent at an appropriate temperature. The compounds of Formula (XXI) are selectively deprotected in a reaction (c) to yield compounds of Formula (XIX). Typical reaction conditions comprise, reaction of the compound of Formula (XXI) with a suitable reagent in a suitable solvent at an appropriate temperature. The compounds of Formulae (VI) and (XIX) are reacted in an amide bond forming reaction (d) to yield compounds of Formula (XIV). Typical reaction conditions comprise, reaction of the amine of Formula (VI) with a carboxylic acid of Formula (XIX) in the presence of a suitable coupling reagent and a suitable base in a suitable solvent at an appropriate temperature. The compounds of Formula (XIV) are selectively deprotected in a reaction (e) to yield compounds of Formula (III). Typical reaction conditions comprise, reaction of the compound of Formula (XIV) with a suitable reagent in a suitable solvent at an appropriate temperature. Similarly, the above process applies to intermediates (XIII), (XIX) and (XVI) in which W is suitably protected throughout step (f) and where reaction (g) leads to concomitant deprotection of W′. Alternatively, intermediate (XVI) may be deprotected in a stepwise manner in that PG₂ is removed in a reaction (h) followed by deprotection of W′ in a separate reaction (i). Preparations according to above scheme are exemplified in the preparations described below for examples SC-085, SC-094 and SC-122.

The preparation of intermediates such as (VI) and (XIII) is known in the literature, e.g. from WO2014 184275, WO2016 075239 and WO2016 075240. All other intermediates and reagents were obtained from commercial sources or were prepared according to procedures known in the literature.

### List of Abbreviations

amu: atomic mass unit, anhyd.: anhydrous, API: atmospheric pressure ionization, aq.: aqueous, Boc: tert-butyloxycarbonyl, cAMP: cyclic adenosine monophosphate, Cbz: benzyloxycarbonyl, DIPEA: N,N-diisopropylethylamine, DIBALH: diisobutylaluminium hydride, DMF: dimethylformamide, DMP: Dess-Martin periodinane, DMSO: dimethyl sulfoxide, dppf: 1,1′-bis(diphenylphosphino)ferrocene, EC50: half maximal effective concentration, EDC: 1-ethyl-3-(3-dimethylaminopropyl)carbodiimide, ESI: electropspray ionization, Et: ethyl, EtOAc: ethyl acetate, Ex.: example, Fmoc: fluorenylmethoxycarbonyl, g: gram, h: hour, h: human, HATU: hexafluorophosphate azabenzotriazole tetramethyl uronium, HOBt: hydroxybenzotriazole, HPLC: high performance liquid chromatography, Hz: hertz, INT: intermediate, L: liter, LCMS: liquid chromatography mass spectrometry, LDA: lithium diisopropylamide, m: mili, M: molar, m: meter, M: mega, Me: methyl, MHz: megahertz, min: minute, MS: mass spectrometry, Ms: mesyl, MTBE: methyl tert-butyl ether, n: nano, nm: nanometer, NMR: nuclear magnetic resonance, Ac: acetyl, PE: petrol ether, PG: protecting group, Ph: phenyl, psi: pounds per square inch, Rt: retention time, sat.: saturated, SC: single compound, SEM: 2-(trimethylsilyl)ethoxymethyl, SPhos: dicyclohexyl(2′,6′-dimethoxy[1,1′-biphenyl]-2-yl)phosphane, SQD: single quadrupole detector, SSTR: somatostatin receptor, TBAF: tetra-N-buityiammnonium fluoride, TBTU: 2-(1H-Benzotriazole-1-yl)-1,1,3,3-tetramethylaminium tetrafluoroborate, Temp: temperature, Tf: triflyl, TFA: trifluoroacetic acid, THF: tetrahydrofuran, Ts: tosyl, UPLC: ultra performance liquid chromatography, UV: ultraviolet.

### Methods

### UPLC-MS/HPLC-MS Methods:

Method A: Instrument: LCMS/MS API 2000 Applied Biosystems; HPLC: Shimadzu Prominence; Column: Zorbax ExtendXBridge C18 (4.6×50 mm, 5μ); Temp: 25° C.; Eluents: A=10 mM NH4OAc in H2O, B=MeCN; Gradient: 0.0 min 90% A→1.5 min 70% A→3.0 min 10% A→4.0 min 10% A→5.0 min 90% A; Flow rate: 1.2 mL/min, Detection: UV 220/260 nm; Ionization: ESI/API; Mass range: 100-800 amu.

Method B: Instrument: Waters ACQUITY SQD; HPLC: Waters ACQUITY UPLC; Column: YMC TRIART C18 (2.1×33 mm, 1.8μ); Temp: 50° C.; Eluents: A=5 mM HCO₂H in H₂O, B=MeCN; Gradient: 0.0 min 98% A→0.75 min 98% A→1.0 min 90% A→2.0 min 2% A→2.25 min 2% A→2.9 min 98% A→3.0 min 98% A; Flow rate: 1.5 mL/min, Detection: UV 210-400 n; Ionization: ESI/API; Mass range: 100-800 amu.

Method C: Instrument: Waters ACQUITY SQD; HPLC: Waters ACQUITY UPLC; Column: Acquity BEH C8 (2.1×50 mm, 1.7μ); Temp: 50° C.; Eluents: A=0.05% HCO₂H in H₂O, B=MeCN; Gradient: 0.0 min 98% A→0.75 min 98% A→1.0 min 90% A→2.0 min 2% A→2.25 min 2% A→2.9 min 98% A→3.0 min 98% A; Flow rate: 1.5 mL/min, Detection: UV 210-400 nm; Ionization: ESI/API; Mass range: 100-800 amu.

Method D: Instrument: Waters ACQUITY SQD 2; HPLC: Waters ACQUITY H Class UPLC; Column: Acquity BEH C18 (2.1×30 mm, 1.7μ); Temp: 50° C.; Eluents: A=5 mM NH₄OAc in H₂O, B=5 mM NH₄OAc in MeCN/H₂O 9:1; Gradient: 0.0 min 98% A→0.5 min 98% A→1.5 min 2% A→2.5 min 2% A→2.75 min 98% A→3.0 min 98% A; Flow rate: 0.5 mL/min, Detection: UV 210/400 nm; Ionization: ESI/API; Mass range: 160-900 amu.

Method E: Instrument: Waters ACQUITY SQD; HPLC: Waters ACQUITY UPLC; Column: XBridge C18 (4.6×50 mm, 5μ); Temp: 50° C.; Eluents: A=5 mM NH₄OAc in H₂O, B=5 mM NH₄OAc in MeCN/H₂O 9:1; Gradient: 0.0 min 98% A→1.0 min 98% A→5.0 min 50% A→8.0 min 10% A→10.0 min 10% A→11.0 min 98% A→12.0 min 98% A; Flow rate: 0.5 mL/min, Detection: UV 210-400 nm; Ionization: ESI/API; Mass range: 100-800 amu.

Method F: Instrument: Waters ACQUITY SQD 2; HPLC: Waters ACQUITY H Class UPLC; Column: Acquity BEH C8 (2.1×50 mm, 1.7μ); Temp: 50° C.; Eluents: A=0.05% HCO₂H in H₂O, B=0.05% HCO₂H in MeCN/H₂O 9:1; Gradient: 0.0 min 95% A→0.75 min 95% A→1.5 min 75% A→3.0 min 5% A→4.0 min 5% A→4.5 min 95% A→5.1 min 95% A; Flow rate: 0.8 mL/min, Detection: UV 210-400 nm; Ionization: ESI/API; Mass range: 160-900 amu.

Method G: Instrument: Agilent SQD; HPLC: Agilent 1260 Infinity-II series; Column: YMC TRIART C18 (2.1×33 mm, 3μ); Temp: 50° C.; Eluents: A=0.01% HCO₂H in H₂O, B=0.01% HCO₂H in MeCN; Gradient: 0.0 min 98% A→0.5 min 98% A→1.0 min 70% A→2.0 min 2% A→2.25 min 2% A→3.0 min 98% A; Flow rate: 1.2 mL/min, Detection: UV 220/260 n; Ionization: ESI/API; Mass range: 100-800 amu.

Method H: Instrument: Waters ACQUITY SQD 2; HPLC: Waters ACQUITY H Class UPLC; Column: XBridge C18 (3.0×50 mm, 3.5μ); Temp: 50° C.; Eluents: A=5 mM NH₄OAc in H₂O, B=5 mM NH₄OAc in MeCN/H₂O 9:1; Gradient: 0.0 min 95% A→0.75 min 95% A→1.0 min 70% A→2.0 min 2% A→2.5 min 2% A→2.75 min 95% A→3.0 min 95% A; Flow rate: 1.2 mL/min, Detection: UV 210-400 n; Ionization: ESI/API; Mass range: 160-900 amu.

Method I: Instrument: Waters ACQUITY SQD 2; HPLC: Waters ACQUITY H Class UPLC; Column: XBridge C18 (3.0×50 mm, 3.5μ); Temp: 50° C.; Eluents: A=5 mM NH₄OAc in H₂O, B=5 mM NH₄OAc in MeCN/H₂O 9:1; Gradient: 0.0 min 95% A→0.75 min 95% A→1.25 min 85% A→2.5 min 30% A→3.75 min 2% A→4.5 min 95% A→5.1 min 95% A; Flow rate: 1.2 mL/min, Detection: UV 220/260 n; Ionization: ESI/API; Mass range: 100-900 amu.

Method J: Instrument: Waters ACQUITY SQD 2; HPLC: Waters ACQUITY H Class UPLC; Column: Waters Acquity UPLC BEH C18 C18 (2.1×50 mm, 1.7μ); Temp: 50° C.; Eluents: A=0.05% HCO₂H in H₂O, B=0.05% HCO₂H in MeCN/H₂O 9:1; Gradient: 0.0 min 95% A→0.75 min 95% A→1.5 min 75% A 3.0 min 5% A→4.0 min 5% A→4.5 min 95% A→5.1 min 95% A; Flow rate: 0.8 mL/min, Detection: UV 210-400 n; Ionization: ESI/API; Mass range: 160-900 amu.

Method K: Instrument: Shimadzu LC-20 AD, MSD:LCMS-2020; Column: XBridge C18 (2.1×50 mm, 5μ); Temp: 40° C.; Eluents: A=10 mM NH₄OAc in H₂O, B=MeCN; Gradient: 0.0 min 95% A→2.5 min 5% A→3.0 min 5% A→3.01 min 95% A→3.5 min 95% A; Flow rate: 1.0 mL/min, Detection: UV 220/254 n; Ionization: ESI/API; Mass range: 100-1800 amu.

Method L: Instrument: Agilent 1200 HPLC, MSD:6120 single quadrupole; Column: XBridge C18 (2.1×50 mm, 5μ); Temp: 40° C.; Eluents: A=10 mM NH₄OAc in H₂O, B=MeCN; Gradient: 0.0 min 95% A→3.4 min 5% A→3.85 min 5% A→3.86 min 95% A→4.5 min 95% A; Flow rate: 0.8 mL/min, Detection: UV 220/254 nm; Ionization: ESI/API; Mass range: 100-2000 amu.

Method M: Instrument: Agilent 1200 HPLC, MSD:1956A single quadrupole; Column: Luna C18 (2.0×50 mm, 5μ); Temp: 40° C.; Eluents: A=0.04% TFA in H₂O, B=0.02% TFA in MeCN; Gradient: 0.0 min 95% A→0.4 min 95% A→3.0 min 5% A→4.0 min 5% A→4.01 min 95% A→4.5 min 95% A; Flow rate: 1.0 mL/min, Detection: UV 220 nm; Ionization: ESI/API; Mass range: 100-2000 amu.

Method N: Instrument: Waters ACQUITY SQD, MSD: single quadrupole; Column: ACQUITY BEH C8 (2.1×50 mm, 1.7μ); Temp: 50° C.; Eluents: A=0.05% HCO₂H in H₂O, B=0.05% HCO₂H in MeCN/H₂O (9:1); Gradient: 0.0 min 95% A→1.0 min 95% A→5.0 min 50% A→8.0 min 10% A→10.0 min 10% A→11.5 min 95% A; Flow rate: 0.8 mL/min, Detection: DAD 220-400 nm; Ionization: electro spray; Mass range: 100-800 amu.

Method O: Instrument: UPLC/MS TOF, Agilent 1290 Infinity II; Column: Waters Acquity HSS T3 (2.1×50 mm, 1.8 μm); Temp: 60° C.; Eluents: A=0.1% HCO₂H in H₂O, B=0.1% HCO₂H in MeCN, Gradient: 0.0 min 99% A→1.7 min 1% A; Flow rate: 2.0 mL/min, Detection: DAD 224-400 nm; Ionization: ESI. Mass range: 100-1000 amu.

Method P: Instrument: UPLC/MS, Waters Acquity; Column: C18 (2.1×50 mm, 1.7 μm); Temp: 22° C., Eluents: A=0.025% NH₄OH in H₂O, B=MeCN; Gradient: 0.0 min 99% A→2.4 min 1% A; Flow rate: 0.75 mL/min, Detection: DAD 245 nm; Ionization: ESI/API, Mass range: 100-1000 amu.

Method Q: Instrument: UPLC/MS, Waters Acquity, Column: C18 (2.1×50 mm, 1.7 μm); Temp: 80° C.; Eluents: A=0.1% HCO₂H in H₂O, B=MeCN; Gradient: 0.0 min 99% A→1.2 min 1% A; Flow rate: 1.5 mL/min; Detection: DAD 245 nm; Ionization ESI/API, Mass range: 100-1000 amu.

### Synthesis of Intermediates:

### Synthesis of INT-001: (3-methylpyridin-2-yl)methanamine

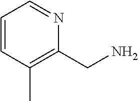

The procedure described below was carried out 100 times in parallel: To a solution of 3-methylpicolinonitrile (10 g, 85 mmol) in EtOH (150 mL) were added Raney-Ni (7.25 g, 85 mmol) and aq. NH₃ solution (25%, 7 mL, 50 mmol) at room temperature and the mixture was heated to 50° C. and stirred for 12 h at that temperature under hydrogen atmosphere (50 psi). The mixture was filtered and the cake washed with EtOH (2×300 mL). The solutions of 100 reactions were combined and concentrated under reduced pressure to obtain the title compound (670 g, 65%) which was used in the next step without further purification. Rt=0.87 min (Method K); ¹H NMR (400 MHz, DMSO-d₆) δ 8.35 (d, 1H), 7.56-7.45 (m, 1H), 7.19-7.08 (m, 1H), 3.78 (s, 2H), 2.26 (s, 3H).

### Synthesis of INT-002: tert-butyl (2-methyl-1-(((3-methylpyridin-2-yl)methyl)amino)-1-oxopropan-2-yl)carbamate

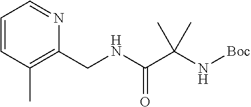 [SMILES: Cc1cccnc1CNC(=O)C(C)(C)NC(=O)OC(C)(C)C]

To a solution of 2-((tert-butoxycarbonyl)amino)-2-methylpropanoic acid (146 g, 720 mmol) in THF (2.2 L) were added amine INT-001 (88 g, 720 mmol), TBTU (213 g, 720 mmol) and Et₃N (218 g, 2.16 mol) and the mixture was purged with nitrogen before it was stirred at room temperature for 16 h under nitrogen atmosphere. The mixture was filtered and concentrated under reduced pressure. The residue was purified by column chromatography (silica, PE/EtOAc 50:1-40:1) to obtain the title compound (222 g, 95%). LCMS m/z=308 [M+H]⁺; ¹H NMR (400 MHz, CDCl₃) δ 8.37 (d, 1H), 8.09 (s, 1H), 7.54-7.45 (m, 1H), 7.15 (t, 1H), 5.20 (s, 1H), 4.51 (d, 2H), 2.32 (s, 3H), 2.13-1.99 (m, 1H), 1.59 (s, 6H), 1.48-1.38 (m, 9H).

### Synthesis of INT-003: tert-butyl (2-(8-methylimidazo[1,5-a]pyridin-3-yl)propan-2-yl)carbamate

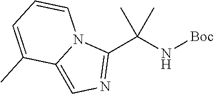 [SMILES: Cc1cccn2c(C(C)(C)NC(=O)OC(C)(C)C)ncc12]

To a solution of carboxamide INT-002 (172 g, 559 mmol) in CH₂Cl₂ (1.72 L) was added a solution of Burgess reagent (133 g, 559 mmol) in CH₂Cl₂ dropwise at 0° C. under nitrogen atmosphere. The mixture was allowed to warm to room temperature and stirred for 16 h before ice water (100 mL) were added and stirred for 10 min. The layers were separated and the aqueous layer extracted with EtOAc (2×100 mL). The combined organic layers were dried over anhyd. Na₂SO₄, filtered and concentrated under reduced pressure. The residue was triturated with MTBE (360 mL), filtered and concentrated under reduced pressure to obtain the title compound (40.0 g, 23%). LCMS m/z=290 [M+H]⁺; ¹H NMR (400 MHz, CDCl₃) δ 8.18-8.06 (m, 1H), 7.42-7.32 (m, 1H), 6.48-6.34 (m, 2H), 5.29-5.16 (m, 1H), 2.46-2.38 (m, 3H), 1.91-1.73 (m, 6H), 1.44-0.76 (m, 9H).

### Synthesis of INT-004: 2-(8-methylimidazo[1,5-a]pyridin-3-yl)propan-2-amine

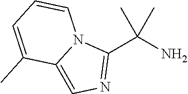

To a solution of carbamate INT-003 (47.0 g, 162 mmol) in MeOH (100 mL) was added HCl (4 M in MeOH, 235 mL, 940 mmol) dropwise at room temperature and the mixture was stirred for 16 h before being filtered and concentrated under reduced pressure. The residue was dissolved in H₂O (100 mL) and the pH adjusted to pH=8-9 with aq. Na₂CO₃ solution. The precipitate was filtered and washed with H₂O (10 mL) and dried under reduced pressure to obtain the title compound (22.0 g, 58%). LCMS m/z=190 [M+H]⁺; ¹H NMR (400 MHz, DMSO-d₆) δ 8.98 (s, 3H), 8.40 (d, 1H), 7.45 (s, 1H), 6.71-6.64 (m, 2H), 2.39 (s, 3H), 1.80 (s, 6H).

### Synthesis of INT-005: 3-iodo-7-methyl-1H-indazole

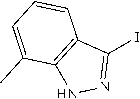

To a solution of 7-methyl-1H-indazole (2.0 g, 15.2 mmol) in DMF (40 mL) were added I₂ (11.5 g, 45.5 mmol) and KOH (4.2 g, 75.8 mmol) at room temperature and the mixture was stirred for 2 h. The mixture was diluted with aq. NaHSO₃ solution (5%, 20 mL) and extracted with EtOAc (3×100 mL). The combined organic layers were washed with H₂O (100 mL), aq. sat. NaCl solution (100 mL), dried over anhyd. Na₂SO₄ and concentrated under reduced pressure to obtain the title compound (3.3 g, 85%). LCMS m/z=258 [M+H]⁺, Rt=2.05 min (Method D).

The following compound was prepared in analogy:

| | | | | LC-MS m/z |
|---|---|---|---|---|
| | | | | [M + H]⁺, Rₜ |
| Intermediate | Structure | Chemical Name | Precursors | [min] (Method) |
| of INT-006 | 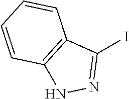 | 3-iodo-1H-indazole | 1H-indazole | 245, 3.09 (A) |

### Synthesis of INT-007: 7-methyl-1H-indazole-3-carbonitrile

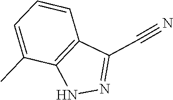

To a degassed solution of indazole INT-005 (2.0 g, 7.75 mmol) in 1,4-dioxane (30 mL) were added Et₃N (3.2 mL, 23.3 mmol), Pd(dppf)Cl₂·CH₂Cl₂ (635 mg, 0.77 mmol), Xantphos (896 mg, 1.55 mmol) and Zn(CN)₂ (1.8 g, 15.5 mmol) at room temperature. The mixture was heated to 100° C. in a sealed tube and stirred for 16 h. The mixture was allowed to cool to room temperature, filtered through a pad of celite and the filter cake was washed with EtOAc (100 mL). The filtrate was concentrated under reduced pressure and the residue was purified by column chromatography (silica, hexane/EtOAc 80:20-78:22) to obtain the title compound (670 mg, 55%). LCMS m/z=158 [M+H]⁺, Rₜ=3.05 min (Method A).

The following compound was prepared in analogy:

| | | | | LC-MS m/z |
|---|---|---|---|---|
| | | | | [M + H]⁺, Rt [min] |
| Intermediate | Structure | Chemical Name | Precursors | (Method) |
| of INT-008 | 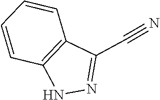 [SMILES: N#Cc1n[nH]c2ccccc12] | 1H-indazole-3-carbonitrile | INT-006 | 144, 2.94 (A) |

### Synthesis of INT-009: 7-methyl-1-((2-(trimethylsilyl)ethoxy)methyl)-1H-indazole-3-carbonitrile

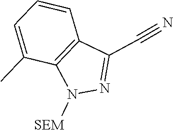 [SMILES: Cc1cccc2c(C#N)nn(COCC[Si](C)(C)C)c12]

To a solution of indazole INT-007 (1.4 g, 8.92 mmol) in DMF (20 mL) was added NaH (535 mg, 13.4 mmol) at 0° C. and the mixture was stirred for 15 min. SEMC1 (1.9 mL, 10.7 mmol) was added and the mixture was allowed to warm to room temperature and stirring was continued for 16 h. The mixture was diluted with EtOAc (200 mL) and washed with H₂O (100 mL) and aq. sat. NaCl solution (100 mL) and dried over anhyd. Na₂SO₄. The mixture was concentrated under reduced pressure and the residue was purified by column chromatography (silica, hexane/EtOAc 92:8) to obtain the title compound (1.5 g, 58%). LCMS m/z=288 [M+H]⁺, ¹H NMR (400 MHz, DMSO-d₆) δ 7.73 (d, 1H), 7.40-7.33 (m, 2H), 5.94 (s, 2H), 3.52 (t, 1H), 2.76 (s, 3H), 0.79 (t, 1H), −0.07 (s, 9H).

### Synthesis of INT-010: 2-(7-methyl-1-((2-(trimethylsilyl)ethoxy)methyl)-1H-indazol-3-yl)propan-2-amine

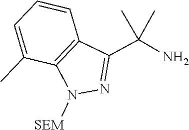 [SMILES: Cc1cccc2c(C(C)(C)N)nn(COCC[Si](C)(C)C)c12]

To CeCl₃ (771 mg, 3.13 mmol) under nitrogen atmosphere was added THF (10 mL) at 0° C. and the mixture was stirred for 2 h. MeLi·LiBr (1.5 M in Et₂O, 2.1 mL, 3.13 mmol) was added to the mixture at −78° C. and stirred at same temperature for 30 min. A solution of nitrile INT-009 (300 mg, 1.04 mmol) in THF (5 mL) was added to the mixture at −78° C. and the mixture was allowed to warm to room temperature and stirred for 16 h. Aq. NaOH solution (50%, 1 mL) and aq. sat. NaHCO₃ solution (1 mL) were added and the mixture and filtered through a pad of celite and the filter cake was washed with THF (10 mL). The filtrate was concentrated under reduced pressure and the residue was purified by column chromatography (silica, CH₂Cl₂/MeOH 3:1) to obtain the title compound (240 mg, 72%). LCMS m/z=320 [M+H]⁺, Rₜ=3.49 min (Method D).

### Synthesis of INT-011: 1-methyl-1H-indazole-3-carbonitrile

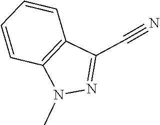

To a solution of indazole INT-008 (1.0 g, 6.94 mmol) in DMF (20 mL) was added 60% NaH (60% in mineral oil, 416 mg, 10.4 mmol) at 0° C. and the mixture was stirred for 15 min. Mel (0.5 mL, 8.33 mmol) was added to the mixture at 0° C. and the mixture was allowed to warm to room temperature and stirred for 3 h. The reaction was quenched by addition of water (50 mL) and extracted with EtOAc (100×2 mL). The combined organic layers were washed with water (100 mL) and aq. sat. NaCl solution (100 mL). The organic layer was dried over anhyd. Na₂SO₄ and concentrated under reduced pressure. The residue was purified by column chromatography (silica, hexane/EtOAc 85:15-80:20) to obtain the title compound (800 mg, 73%). ¹H NMR (400 MHz, CDCl₃) δ 7.83 (d, 1H), 7.51 (d, 2H), 7.35 (m, 1H), 4.16 (s, 3H); Rt=3.12 min (Method A).

### Synthesis of INT-012: 2-(1-methyl-1H-indazol-3-yl)propan-2-amine

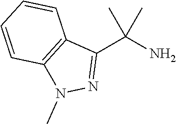

To a solution of nitrile INT-011 (1.4 g, 8.91 mmol) in PhMe (50 mL) was added MeMgBr (3 M in Et₂O, 14.9 mL, 44.6 mmol) and mixture was heated to reflux and stirred for 16 h. The reaction was quenched by addition of aq. sat. NH₄Cl (100 mL) and extracted with EtOAc (2×100 mL). The combined organic layers were washed with aq. sat. NaCl solution (100 mL), dried over anhyd. Na₂SO₄ and concentrated under reduced pressure. The residue was purified by column chromatography (silica, CH₂Cl₂/MeOH 97:3-95:5 to obtain the title compound (750 mg, 46%). ¹H NMR (400 MHz, CDCl₃) δ 7.90 (d, 1H), 7.43-7.25 (m, 2H), 7.10 (t, 1H), 4.00 (s, 3H), 1.66 (s, 6H). Rt=2.03 min (Method A).

### Synthesis of INT-013: (3-chloropyridin-2-yl)methanamine

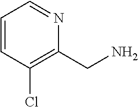

To a solution of 3-chloropicolinonitrile (75 g, 541 mmol) in EtOH (850 mL) and NH₃ (25% aq. solution, 350 mL) was added Raney Ni (40 g, wet) at room temperature. The mixture was stirred for 1.5 h under H₂ atmosphere (1.38 bar) in Parr shaker before more Raney Ni (30 g, wet) was added and stirring was continued for 2 h. The mixture was filtered through a pad of celite and washed with CH₂Cl₂/MeOH (4:1, 2 L). The filtrate was concentrated under reduced pressure and the residue was diluted with CH₂Cl₂/MeOH (9:1, 500 mL), dried over anhyd. Na₂SO₄ and concentrated under reduced pressure to obtain the title compound (67 g, 86%) which was used in the next step without further purification. ¹H NMR (400 MHz, CDCl₃) δ 8.45 (m, 1H), 7.63 (m, 1H), 7.14 (dd, 1H), 4.08 (s, 2H).

### Synthesis of INT-014: 2-(1,3-dioxoisoindolin-2-yl)-2-methylpropanoic acid

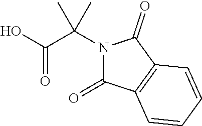 [SMILES: CC(C)(C(=O)O)N1C(=O)c2ccccc2C1=O]

To a solution of 2-amino-2-methylpropanoic acid (100 g, 0.97 mol) and isobenzofuran-1,3-dione (172.5 g, 1.16 mol) in PhMe (1 L) was added Et₃N (162.5 mL, 1.16 mol) at room temperature and the mixture was stirred at 120° C. for 48 h removing water using a Dean-Stark apparatus. The mixture was allowed to cool to room temperature and concentrated under reduced pressure. The residue was poured into H₂O (1 L) and was extracted with EtOAc (3×1.2 L). The combined organic layers were washed with aq. sat. NaCl solution (2×1.5 L), dried over anhyd. Na₂SO₄ and concentrated under reduced pressure. The residue was triturated with hexane/EtOAc (9:1) and filtered to obtain the title compound (134 g, 59%). LCMS m/z=234 [M+H]⁺; ¹H NMR (400 MHz, DMSO-d₆) δ 12.98 (s, 1H), 7.86 (s, 4H), 1.71 (s, 6H).

### Synthesis of INT-015: N-((3-chloropyridin-2-yl)methyl)-2-(1,3-dioxoisoindolin-2-yl)-2-methylpropanamide

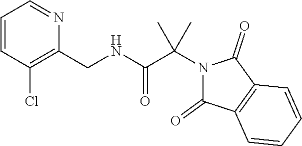 [SMILES: CC(C)(C(=O)NCc1ncccc1Cl)N1C(=O)c2ccccc2C1=O]

To a solution of carboxylic acid INT-014 (100 g, 429 mmol) and amine INT-013 (73.5 g, 514 mmol) in DMF (1 L) were added EDC HCl (123.5 g, 643 mmol) and HOBt (87 g, 643 mmol) at 0° C. The mixture was stirred for 15 min at 0° C. before DIPEA (298.5 mL, 1.7 mol) was added and the mixture was stirred at room temperature for 16 h. The mixture was poured into ice water (3.5 L) and was extracted with EtOAc (3×1.2 L). The combined organic layers were washed with aq. sat. NaHCO₃ solution (1.5 L), aq. sat. NH₄Cl solution (1.5 L) and aq. sat. NaCl solution (2×1.5 L), dried over anhyd. Na₂SO₄ and concentrated under reduced pressure. The residue was triturated with MTBE and filtered to obtain the title compound (112 g, 73%). LCMS m/z=358 [M+H]⁺; ¹H NMR (400 MHz, DMSO-d₆) δ 8.43 (m, 2H), 7.88 (m, 1H), 7.81 (s, 4H), 7.33 (dd, 1H), 4.43 (d, 2H), 1.71 (s, 6H).

### Synthesis of INT-016: 2-(2-(8-chloroimidazo[1,5-a]pyridin-3-yl)propan-2-yl)isoindoline-1,3-dione

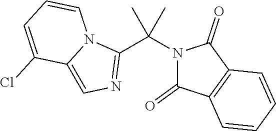 [SMILES: CC(C)(c1ncc2c(Cl)cccn12)N1C(=O)c2ccccc2C1=O]

To a solution of carboxamide INT-015 (102 g, 285 mmol) in PhMe (1.53 L) was added POCl₃ (80 mL, 855 mmol) at room temperature. The mixture was heated to 120° C. and stirred for 16 h at that temperature. The mixture was allowed to cool to room temperature, poured into ice water and the mixture was basified with sat. aq. NaHCO₃ solution. The mixture was extracted with CH₂Cl₂/MeOH (9:1, 3×1 L) and the combined organic layers were washed with aq. sat. NaCl solution (1.2 L), dried over anhyd. Na₂SO₄ and concentrated under reduced pressure. The residue was purified by column chromatography (silica, hexane/EtOAc 1:1) followed by trituration with pentane to afford the title compound (54 g, 56%). LCMS m/z=340 [M+H]⁺; ¹H NMR (400 MHz, DMSO-d₆) (8.22 (d, 1H), 7.80 (m, 4H), 7.45 (s, 1H), 6.91 (d, 1H), 6.50 (t, 1H), 2.05 (s, 6H).

### Synthesis of INT-017: 2-(8-chloroimidazo[1,5-a]pyridin-3-yl)propan-2-amine

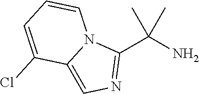

A suspension of phthalimide INT-016 (54.0 g, 158 mmol) in methyl amine (40% aq. solution, 270 mL) was stirred at 80° C. for 16 h in a sealed round bottom flask. The mixture was allowed to cool to room temperature and was diluted with EtOAc (600 mL), washed with cold H₂O (3×90 mL) and aq. sat. NaCl solution (100 mL) and dried over anhyd. Na₂SO₄. The mixture was concentrated under reduced pressure to obtain the title compound (31.5 g, 95%) which was used in the next step without further purification. LCMS m/z=210 [M+H]⁺; ¹H NMR (400 MHz, DMSO-d₆) δ 9.03 (d, 1H), 7.33 (s, 1H), 6.92 (d, 1H), 6.59 (t, 1H), 2.14 (s, 2H), 1.55 (s, 6H).

### Synthesis of INT-018: 1-(3-methylpyridin-2-yl)ethan-1-ol

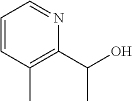

To a solution of 3-methylpicolinaldehyde (5.0 g, 41.3 mmol) in THF (100 mL) was added MeMgBr (3 M in Et₂O, 27.5 mL, 82.5 mmol) at 0° C. and the mixture was allowed to warm to room temperature and stirred for 4 h. The reaction was quenched by addition of aq. sat. NH₄Cl solution (50 mL) and extracted with EtOAc (500 mL). The organic layer was dried over anhdy. Na₂SO₄ and concentrated under reduced pressure. The residue was purified by column chromatography (silica, hexane/EtOAc 1:1) to obtain the title compound (5.3 g, 94%). LCMS m/z=138 [M+H]⁺, Rₜ=2.05 min (Method A).

### Synthesis of INT-019: 1-(3-methylpyridin-2-yl)ethyl methanesulfonate

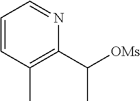

To a solution of alcohol INT-018 (10.2 g, 74.45 mmol) in CH₂Cl₂ (100 mL) was added Et₃N (15.5 mL, 11.7 mmol) followed by the addition of MsCl (6.91 mL, 89.3 mmol) at 0° C. and the mixture was allowed to warm to room temperature and stirred for 2 h. The reaction was quenched by addition of aq. NaHCO₃ solution (50 mL) and extracted with CH₂Cl₂ (500 mL). The organic layer was washed with aq. sat. NaCl solution (100 mL), dried over anhyd. Na₂SO₄ and concentrated under reduced pressure to obtain the title compound (16 g, quantitative) which was used in the next step without further purification. LCMS m/z=216 [M+H]⁺, Rₜ=2.49 min (Method A).

### Synthesis of INT-020: 2-(1-azidoethyl)-3-methylpyridine

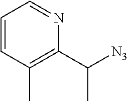

To a solution of methanesulfonate INT-019 (16.0 g, 74.4 mmol) in DMF (100 mL) was added NaN₃ (7.4 g, 113 mmol) at room temperature and the mixture was heated and stirred at 50° C. for 1 h. The mixture was allowed to cool to room temperature before aq. NaHCO₃ solution (100 mL) was added and the mixture extracted with EtOAc (500 mL). The organic layer was washed with aq. sat. NaCl solution (100 mL), dried over anhyd. Na₂SO₄ and concentrated under reduced pressure to obtain the title compound (12 g, 99%) which was used in the next step without further purification. LCMS m/z=163 [M+H]⁺, Rₜ=3.15 min (Method A).

### Synthesis of INT-021: 1-(3-methylpyridin-2-yl)ethan-1-amine

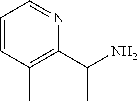

To a solution of azide INT-020 (12.0 g, 73.6 mmol) in THF/H₂O (10:2, 120 mL) was added PPh₃ (38.6 g, 147 mmol) at room temperature and the mixture was stirred for 16 h. The mixture was concentrated under reduced pressure, the residue was triturated with Et₂O (50 mL) and the precipitated solid was filtered. The filtrate was concentrated under reduced pressure and purified by column chromatography (silica, CH₂Cl₂/MeOH 1:0-8:2) to obtain the title compound (7.5 g, 75%). LCMS m/z=137 [M+H]⁺, Rₜ=1.22 min (Method A).

### Synthesis of INT-022: tert-butyl (2-methyl-1-oxo-1-(((3-(trifluoromethyl)pyridin-2-yl)methyl)amino)propan-2-yl)carbamate

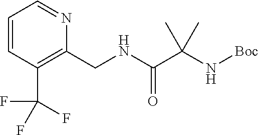 [SMILES: CC(C)(C)OC(=O)NC(C)(C)C(=O)NCc1ncccc1C(F)(F)F]

To a solution of 2-((tert-butoxycarbonyl)amino)-2-methylpropanoic acid (1.39 g, 6.87 mmol) in DMF (20 mL) were added EDC HCl (2.39 g, 12.5 mmol), HOBt (1.26 g, 9.37 mmol) and DIPEA (3.26 mL, 18.8 mmol) followed by addition of (3-(trifluoromethyl)pyridin-2-yl)methanamine (1.1 g, 6.25 mmol) at 0° C. The mixture was allowed to warm to room temperature and stirred for 16 h before cold water (50 mL) was added and the mixture was extracted with EtOAc (2×100 mL). The combined organic layers were washed with aq. sat. NaCl solution (50 mL), dried over anhyd. Na₂SO₄ and concentrated under reduced pressure. The residue was purified by column chromatography (silica, hexane/EtOAc 1:1-4:6) to obtain the title compound (1.1 g, 44%). LCMS m/z=362 [M+H]⁺, Rₜ=3.17 min (Method A).

The following compound was prepared in analogy:

| | | | | LC-MS m/z |
|---|---|---|---|---|
| Interme- | | | | [M + H]⁺, Rt |
| diate | Structure | Chemical Name | Precursors | [min] (Method) |
| INT- 023 | 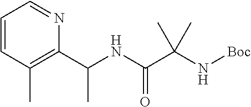 [SMILES: Cc1cccnc1C(C)NC(=O)C(C)(C)NC(=O)OC(C)(C)C] | rac-tert-butyl (2-methyl-1- ((1-(3-methylpyridin-2- yl)ethyl)amino)-1-oxopro- pan-2-yl)carbamate | INT-021 | 322, 2.98 (A) |

### Synthesis of INT-024: 3-cyclopropylpicolinonitrile

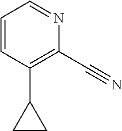 [SMILES: N#Cc1ncccc1C1CC1]

To a stirred solution of 3-bromopicolinonitrile (1.0 g, 5.46 mmol) in PhMe/H₂O (100:1, 66 mL) were added cyclopropylboronic acid (0.61 g, 7.1 mmol), tricyclohexylphosphine (0.23 g, 0.82 mmol) and anhyd. K₃PO₄ (4.06 g, 19.1 mmol) and the mixture was purged with argon for 10 min. Pd(OAc)₂ (98 mg, 0.44 mmol, 0.08 equiv.) was added under inert atmosphere and the mixture was heated to 100° C. and stirred for 16 h at that temperature. The mixture was allowed to cool to room temperature, diluted with H₂O (50 mL) and extracted with EtOAc (2×30 mL). The combined organic layers were dried with anhyd. Na₂SO₄ and concentrated under reduced pressure. The residue was purified by column chromatography (silica, hexane/EtOAc 9:1) obtain the title compound (725 mg, 92%). LCMS m/z=145 [M+H]⁺, Rₜ=3.01 min (Method A).

### Synthesis of INT-025: (3-cyclopropylpyridin-2-yl)methanamine

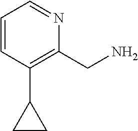 [SMILES: NCc1ncccc1C1CC1]

To a solution of nitril INT-024 (725 mg, 5.03 mmol) in MeOH (15 mL) was added Raney Ni (360 mg) and NH₃ (25% aq. solution, 2.0 mL) and the mixture was stirred under hydrogen atmosphere (1 atm) at room temperature for 16 h. The mixture was filtered through a pad of celite and the filtrate was concentrated under reduced pressure to obtain the title compound (750 mg, quantitative) which was used without further purification in the next step. LCMS m/z=149 [M+H]⁺, Rₜ=1.60 min (Method A).

### Synthesis of INT-163: (3-methoxypyridin-2-yl)methanamine

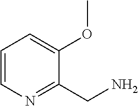

To a stirred solution of 3-methoxypicolinonitrile (300 mg, 2.2 mmol) in PhMe (4.5 mL) under nitrogen was slowly added DIBALH (1M in PhMe, 11.2 mL, 11.2 mmol) and the mixture was stirred at 0° C. for 30 min. The mixture was diluted with aq. NaOH solution (1 M, 11 mL) at 0° C., methanol (4.5 mL) was added and the mixture was allowed to warm to room temperature. The mixture was filtered and the solid residue was washed with methanol (50 mL). The filtrate was concentrated under reduced pressure to afford the title compound (290 mg, 94%) which was used in the next step without further purification. LCMS m/z=139 [M+H]⁺, Rₜ=1.09 min (Method P).

### Synthesis of INT-026: tert-butyl (1-(((3-cyclopropylpyridin-2-yl)methyl)amino)-2-methyl-1-oxopropan-2-yl)carbamate

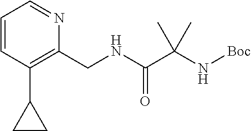 [SMILES: CC(C)(C)OC(=O)NC(C)(C)C(=O)NCc1ncccc1C1CC1]

To a solution of 2-((tert-butoxycarbonyl)amino)-2-methylpropanoic acid (1.13 g, 5.56 mmol) in DMF (20 mL) were added amine INT-025 (0.75 g, 5.06 mmol) HATU (2.31 g, 6.07 mmol) and DIPEA (2.65 mL, 15.18 mmol) at 0° C. and the mixture was allowed to warm to room temperature and stirred for 16 h. The reaction was quenched by addition of H₂O (50 mL) and the mixture extracted with EtOAc (2×100 mL). The organic layer was washed with aq. sat. NaCl solution (25 mL) and dried over anhyd. Na₂SO₄. The mixture was concentrated under reduced pressure and the residue was purified by column chromatography (silica, hexane/EtOAc 3:7) to obtain the title compound (1.1 g, 65%). LCMS m/z=334 [M+H]⁺, Rₜ=3.04 min (Method A).

The following compound was prepared in analogy:

| | | | | LC-MS m/z |
|---|---|---|---|---|
| Interme- | | | Precur- | [M + H]⁺, Rt [min] |
| diate | Structure | Chemical Name | sors | (Method) |
| INT- 164 | 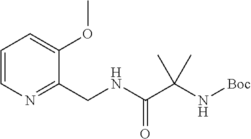 [SMILES: COc1cccnc1CNC(=O)C(C)(C)NC(=O)OC(C)(C)C] | tert-butyl (1-(((3- methoxypyridin-2-yl) methyl)amino)- 2-methyl- 1-oxopropan-2-yl) carbamate | INT- 163 | 324, 0.55 (O) |

### Synthesis of INT-027: tert-butyl (2-(8-(trifluoromethyl)imidazo[1,5-a]pyridin-3-yl)propan-2-yl)carbamate

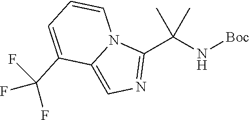 [SMILES: CC(C)(C)OC(=O)NC(C)(C)c1ncc2c(C(F)(F)F)cccn12]

To a solution of carboxamide INT-022 (2.0 g, 5.54 mmol) in CH₂Cl₂ (60 mL) was added Burgess reagent (3.29 g, 13.9 mmol) portion wise at 0° C. and the mixture was allowed to warm to room temperature and stirred for 48 h. The mixture was diluted with CH₂Cl₂ (200 mL), washed with H₂O (100 mL) and aq. sat. NaCl solution (100 mL). The organic layer was dried over anhyd. Na₂SO₄ and the filtrate was concentrated under reduced pressure. The residue was purified by column chromatography (silica, hexane/EtOAc 1:1-4:6) to obtain the title compound (300 mg, 16%). LCMS m/z=344 [M+H]⁺, Rₜ=3.27 min (Method A).

The following compounds were prepared in analogy:

| | | | | LC-MS m/z |
|---|---|---|---|---|
| Interme- | | | Precur- | [M + H]⁺, Rt [min] |
| diate | Structure | Chemical Name | sors | (Method) |
| INT- 028 | 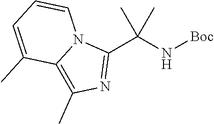 [SMILES: Cc1cccn2c(C(C)(C)NC(=O)OC(C)(C)C)nc(C)c12] | tert-butyl (2-(1,8- dimethylimidazo[1,5-a] pyridin-3-yl)propan-2-yl) carbamate | INT- 023 | 304, 3.23 (A) |
| INT- 029 | 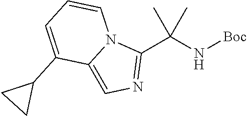 [SMILES: CC(C)(C)OC(=O)NC(C)(C)c1ncc2c(C3CC3)cccn12] | tert-butyl (2-(8-cyclo- propylimidazo[1,5-a] pyridin-3-yl)propan-2- yl)carbamate | INT- 026 | 316, 3.19 (A) |
| INT- 165 | 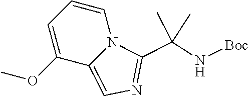 [SMILES: COc1cccn2c(C(C)(C)NC(=O)OC(C)(C)C)ncc12] | tert-butyl (2-(8-methoxy- imidazo[1,5-a]pyridin-3- yl)propan-2-yl)carba- mate | INT- 164 | 306, 0.57 (O) |

### Synthesis of INT-030: 2-(8-(trifluoromethyl)imidazo[1,5-a]pyridin-3-yl)propan-2-amine

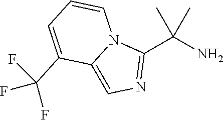 [SMILES: CC(C)(N)c1ncc2c(C(F)(F)F)cccn12]

To a solution of carbamate INT-027 (300 mg, 0.87 mmol) in CH₂Cl₂ (10 ml) was added HCl (4 M in 1,4-dioxane, 10 ml) at 0° C. and the mixture was allowed to warm to room temperature and stirred for 16 h. The mixture was concentrated under reduced pressure and the residue was washed with pentane to obtain the title compound (300 mg, quantitative) as its HCl salt. This material was used without further purification in the next step. LCMS m/z=244 [M+H]⁺; ¹H NMR (400 MHz, DMSO-d₆) δ 9.04-8.95 (m, 2H), 8.80 (d, 1H), 7.57 (s, 1H), 7.40 (d, 1H), 6.91-6.87 (t, 1H), 3.79 (s, 1H), 3.70-3.68 (m, 1H), 1.80 (s, 6H).

The following compounds were prepared in analogy:

| Inter- | | | Precur- | LC-MS m/z [M + H]⁺, Rt |
|---|---|---|---|---|
| mediate | Structure | Chemical Name | sors | [min] (Method) |
| INT- 031 | 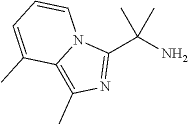 | 2-(1,8-dimethylimi- dazo[1,5-a]pyridin-3- yl)propan-2-amine | INT- 028 | 204, 2.55 (A) |
| INT- 032 | 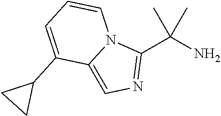 [SMILES: CC(C)(N)c1ncc2c(C3CC3)cccn12] | 2-(8-cyclopropyl- imidazo[1,5-a]pyridin-3-yl) propan-2-amine | INT- 029 | 216, 2.53 (A) |

### Synthesis of INT-146: rac-tert-butyl (1-(2,3-difluorophenyl)-1-hydroxy-2-methylpropan-2-yl)carbamate

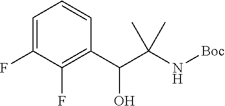 [SMILES: CC(C)(C)OC(=O)NC(C)(C)C(O)c1cccc(F)c1F]

To a solution of tert-butyl N-(1-hydroxy-2-methylpropan-2-yl) carbamate (10.0 g, 53 mmol) in CH₂Cl₂ (350 mL) was added DMP (45 g, 106 mmol) at 0° C. and the mixture was stirred at room temperature for 16 h. To the mixture was added a mixture of aq. NaHCO₃ solution (200 mL) and aq. Na₂S₂O₃ solution (200 mL) at 0° C. and stirred at same temperature for 1 h. The mixture was filtered through sintered funnel and washed with CH₂Cl₂ (500 mL). The organic layer was separated, washed with water (300 mL) and sat. aq. NaCl solution (300 mL), dried over anhyd. Na₂SO₄ and concentrated under reduced pressure. The crude material was purified by column chromatography (silica, hexane/EtOAc 1:0-7:3) to yield tert-butyl (2-methyl-1-oxopropan-2-yl)carbamate which was directly used in the next step.

To a solution of 1,2-difluorobenzene (5.5 g, 48 mmol) in THF (40 mL) was added n-BuLi (1.8M in hexane, 31 mL, 56 mmol) at −78° C. and the mixture was stirred for 2 h, followed by dropwise addition of freshly prepared tert-butyl N-(2-methyl-1-oxopropan-2-yl)carbamate (3.0 g, 16 mmol) in THF (10 mL) at −78° C. The mixture was stirred for 1 h before saturated NH₄Cl solution (100 mL) was added and the mixture was extracted with EtOAc (2×500 mL). The combined organic layers were washed with sat. aq. NaCl solution (100 mL), dried over anhyd. Na₂SO₄ and concentrated under reduced pressure. The crude material was purified by column chromatography (silica, hexane/EtOAc 1:0-10:1) to afford the title product (2.7 g, 56%). LCMS m/z=302 [M+H]⁺, Rₜ=3.53 min (Method A).

### Synthesis of INT-147: tert-butyl (1-(2,3-difluorophenyl)-2-methyl-1-oxopropan-2-yl)carbamate

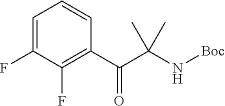 [SMILES: CC(C)(C)OC(=O)NC(C)(C)C(=O)c1cccc(F)c1F]

To a solution of alcohol INT-146 (2.6 g, 8.6 mmol) in CH₂Cl₂ (50 mL) was added DMP (4.0 g, 9.5 mmol) at 0° C. and the mixture was stirred at room temperature for 16 h. To the mixture were added aq. NaHCO₃ solution (200 mL) and aq. Na₂S₂O₃ solution (100 mL) at 0° C., the mixture was filtered through pad of celite pad and the residue washed with CH₂Cl₂ (300 mL). The organic layer was washed with water (300 mL), sat. aq. NaCl. solution (300 mL) and dried over anhyd. Na₂SO₄ and passed through silica bed. The crude material was concentrated under reduced pressure to yield the title product (1.8 g, 69%). LCMS m/z=300 [M+H]⁺, Rₜ=3.53 min (Method A).

### Synthesis of INT-148: tert-butyl (2-(7-fluoro-1H-indazol-3-yl)propan-2-yl)carbamate

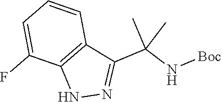 [SMILES: CC(C)(C)OC(=O)NC(C)(C)c1n[nH]c2c(F)cccc12]

To a solution of ketone INT-147 (1.0 g, 3.3 mmol) in EtOH (10 mL) was added N₂H₄·H₂O (0.3 mL, 5.0 mmol) at room temperature and the reaction mixture was heated under microwave irradiation at 140° C. for 1.5 h. The mixture was diluted with water and extracted with EtOAc (2×200 mL). The combined organic layers were washed with sat. aq. NaCl solution (50 mL), dried over anhyd. Na₂SO₄ and concentrated under reduced pressure. The crude material was purified by column chromatography (silica, hexane/EtOAc 1:0-7:3) to afford the title product (0.5 g, 51%). LCMS m/z=294 [M+H]⁺, Rₜ=3.22 min (Method A).

### Synthesis of INT-149: 2-(7-fluoro-1H-indazol-3-yl)propan-2-amine

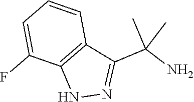

To a solution of carbamate INT-148 (6.0 g, 20 mmol) in CH₂Cl₂ (40 mL) was added HCl in 1,4-dioxane (4 M, 30 mL) at 0° C. and the mixture was stirred at room temperature for 2 h. The mixture was concentrated under reduced pressure and the crude material was neutralized with sat. NaHCO₃ solution and extracted with EtOAc (2×300 mL). The combined organic layers were dried over anhyd. Na₂SO₄ and concentrated under reduced pressure. The crude product was purified by preparative HPLC (YMC-Actus Triart C18 (250×30 mm, 5μ), elution with 20 mM NH₄HCO₃ in H₂O/MeCN (80:20-5:95) at ambient temperature) to afford the title compound (1.5 g, 31%). LCMS m/z=194 [M+H]⁺, Rₜ=2.05 min (Method A).

### Synthesis of INT-150: rac-tert-butyl (1-(3-chloro-2-fluorophenyl)-1-hydroxy-2-methylpropan-2-yl)carbamate

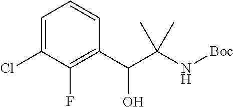 [SMILES: CC(C)(C)OC(=O)NC(C)(C)C(O)c1cccc(Cl)c1F]

To a stirred solution of 1-chloro-2-fluorobenzene (16.6 g, 128 mmol) in THF (150 mL) was added n-BuLi (1.8 M in hexane, 44.3 mL, 79 mmol) dropwise at −78° C.:. The mixture was stirred at −78° C. for 2 h followed by addition of tert-butyl (2-methyl-1-oxopropan-2-yl)amino formate (6.0 g, 32 mmol) at −78° C. The mixture was stirred at same temperature for another 1 h and subsequently sat. aq. NH₄Cl solution was added and the mixture was extracted with EtOAc (3×200 mL). The combined organic layers were dried over anhyd. Na₂SO₄ and concentrated under reduced pressure. The crude material was purified by column chromatography (silica, hexane/EtOAc 1:0-85:15) to obtain the title compound (8 g, 79%). LCMS m/z=318 [M+H]⁺, Rₜ=3.65 min (Method A).

### Synthesis of INT-151: tert-butyl (1-(3-chloro-2-fluorophenyl)-2-methyl-1-oxopropan-2-yl)carbamate

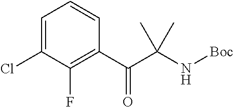 [SMILES: CC(C)(C)OC(=O)NC(C)(C)C(=O)c1cccc(Cl)c1F]

To a stirred solution of alcohol INT-150 (8 g, 25 mmol) in CH₂Cl₂ (160 mL) was added DMP (12 g, 28 mmol) at 0° C. The mixture was stirred at 25° C. for 12 h and subsequently the pH was adjusted to 8 by adding aq. NaHCO₃. The organic layer was separated and the aqueous layer was extracted with EtOAc (2×250 mL). The combined organic layers were dried over anhyd. Na₂SO₄ and concentrated under reduced pressure. The crude material was purified by column chromatography (silica, hexane/EtOAc 1:0-8:2) to yield the title compound (8 g, 98%). LCMS m/z=316 [M+H]⁺, Rₜ=3.65 min (Method A).

### Synthesis of INT-152: tert-butyl (1-(3-chloro-2-fluorophenyl)-1-(hydroxyimino)-2-methylpropan-2-yl)carbamate

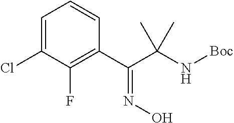 [SMILES: CC(C)(C)OC(=O)NC(C)(C)/C(=N\O)c1cccc(Cl)c1F]

To a solution of ketone INT-151 (8.1 g, 26 mmol) in EtOH (400 mL) were added hydroxylamine-HCl (8.9 g, 127 mmol) and NaOAc (10.5 g, 128 mmol) at room temperature. The mixture was heated and stirred at 90° C. for 16 h. The mixture was concentrated under reduced pressure and the residue was diluted with water before it was extracted with EtOAc. The combined organic layers were dried over anhyd. Na₂SO₄ and concentrated under reduced pressure. The crude material was purified by column chromatography (silica, hexane/EtOAc 1:0-7:3) to obtain the title compound (6.5 g, 77%). LCMS m/z=331 [M+H]⁺, Rₜ=3.45 min (Method A).

### Synthesis of INT-153: tert-butyl (2-(7-chlorobenzo[d]isoxazol-3-yl)propan-2-yl)carbamate

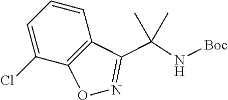 [SMILES: CC(C)(C)OC(=O)NC(C)(C)c1noc2c(Cl)cccc12]

To a solution of oxime INT-152 (4.8 g, 14.46 mmol, 1 equiv.) in THF (100 mL) was added t-BuOK (4.8 g, 43 mmol) at room temperature and the mixture was stirred for 2 h. The mixture was diluted with ice water and extracted with EtOAc. The combined organic layers were dried over anhyd. Na₂SO₄ and concentrated under reduced pressure. The crude material was purified by column chromatography (silica, hexane/EtOAc 1:0-7:3) to the title product (3 g, 67%). LCMS m/z=311 [M+H]⁺, Rₜ=3.60 min (Method A).

### Synthesis of INT-156: tert-butyl (2-(7-chloro-1-methyl-1H-indazol-3-yl)propan-2-yl)carbamate

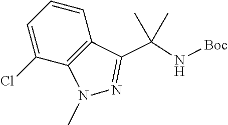 [SMILES: Cn1nc(C(C)(C)NC(=O)OC(C)(C)C)c2cccc(Cl)c21]

To a solution of ketone INT-151 (6.0 g, 18 mmol) in EtOH (50 mL) was added methyl hydrazine (20 mL, 378 mmol) at room temperature. The mixture was stirred at 80° C. for 16 hours and was subsequently concentrated under reduced pressure. The crude material was purified by column chromatography (silica, hexane/EtOAc 1:0-5:1) to afford the title compound (3 g, 49%). LCMS m/z=324 [M+H]⁺, Rₜ=3.71 min (Method A).

### Synthesis of INT-154: tert-butyl (2-(7-methylbenzo[d]isoxazol-3-yl)propan-2-yl)carbamate

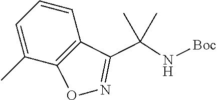 [SMILES: Cc1cccc2c(C(C)(C)NC(=O)OC(C)(C)C)noc12]

To a solution of benzisoxazole INT-153 (3.5 g, 11 m mol), in PhMe (200 mL) were added methylboronic acid (4.3 g, 72 mmol) and K₃PO₄ (12 g, 56 mmol). The mixture was degassed and purged with nitrogen for 15 minutes followed by addition of SPhos (0.9 g, 2.2 mmol) and Pd(OAc)₂ (500 mg, 2.2 mmol). The mixture was stirred at 120° C. for 4 hours under nitrogen atmosphere and subsequently filtered through a pad of celite. The filtrate was concentrated under reduced and the crude material was purified by column chromatography (silica, hexane/EtOAc 1:0-11) to yield the title compound (2 g, 61%). LCMS m/z=291 [M+H]⁺, Rₜ=3.56 min (Method A).

The following compound was prepared in analogy:

| | | | | LC-MS m/z |
|---|---|---|---|---|
| Interme- | | | | [M + H]⁺, Rt |
| diate | Structure | Chemical Name | Precursors | [min] (Method) |
| INT- 157 | 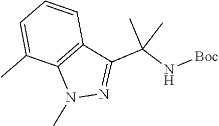 [SMILES: Cc1cccc2c(C(C)(C)NC(=O)OC(C)(C)C)nn(C)c12] | tert-butyl (2-(1,7- dimethyl-1H-indazol-3-yl) propan-2-yl)carbamate | INT-156 | 304, 3.52 (A) |

### Synthesis of INT-159: 3-chloro-2-hydrazineylpyridine

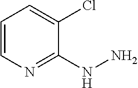

A mixture of 3-chloro-2-fluoropyridine (15.0 g, 114 mmol) and hydrazine hydrate (22.2 mL, 456 mmol) in ethanol (150 mL) was stirred at 90° C. for 16 h. The mixture was concentrated under reduced pressure and the resulting solid was triturated with hexane to obtain the title compound (16 g, 98%) which was used in the next step without further purification. LCMS m/z=144 [M+H]⁺, Rₜ=1.91 min (Method A).

### Synthesis of INT-160: tert-butyl (1-(2-(3-chloropyridin-2-yl)hydrazineyl)-2-methyl-1-oxopropan-2-yl)carbamate

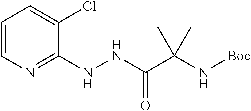 [SMILES: CC(C)(C)OC(=O)NC(C)(C)C(=O)NNc1ncccc1Cl]

To a stirred solution of hydrazine INT-159 (10 g, 143 mmol) in DMF (100 mL) were added 2-((tert-butoxycarbonyl)amino)-2-methylpropanoic acid (12.8 g, 62 mmol), HATU (31.8 g, 83 mmol) and DIPEA (36.4 mL, 208 mmol) at 0° C. and the mixture was stirred at room temperature for 16 h. Aqueous NaHCO₃ solution was added and the mixture was extracted with EtOAc (2×50 mL). The combined organic layers were washed with aq. NH₄Cl solution and aq. sat. NaCl solution. The mixture was dried over anhyd. Na₂SO_{d} and concentrated under reduced pressure. The crude material was triturated with MTBE to the title compound (19 g, 83%) which was used in the next step without further purification. LCMS m/z=329 [M+H]⁺, Rₜ=2.97 min (Method A).

### Synthesis of INT-161: tert-butyl (2-(8-chloro-[1,2,4]triazolo[4,3-a]pyridin-3-yl)propan-2-yl)carbamate

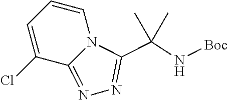 [SMILES: CC(C)(C)OC(=O)NC(C)(C)c1nnc2c(Cl)cccn12]

To a stirred solution of hydrazide INT-160 (15.0 g, 328 mmol) in CH₂Cl₂ (300 mL) was added Burgess reagent (32.7 g, 136 mmol) at 0° C. and the mixture was stirred at room temperature for 3 days. The mixture was diluted with CH₂Cl₂ (500 mL) and washed with water (200 mL) and sat. aq. NaCl solution (200 mL). The organic layer was concentrated under reduced pressure and the crude material was purified by column chromatography (silica, hexane/aceton 1:0-3:7) to obtain the title compound (3 g, 21%). LCMS m/z=311 [M+H]⁺, Rₜ=2.64 min (Method A).

### Synthesis of INT-155: 2-(7-methylbenzo[d]isoxazol-3-yl)propan-2-amine

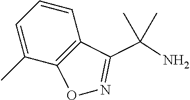

To a solution of carbamate INT-154 (2.3 g, 7.9 mmol) in CH₂Cl₂ (50 mL) was added HCl (1 M in 1,4-dioxane, 23 mL) at 0° C. The mixture was stirred at room temperature for 1 h and concentrated under reduced pressure. The crude material was neutralized with sat. NaHCO₃ solution and extracted with EtOAc (200 mL). The combined organic layers were dried over anhyd. Na₂SO₄ and concentrated under reduced pressure to afford the title product which was used without further purification (2.4 g, 85%). LCMS m/z=191 [M+H]⁺, Rₜ=2.07 min (Method C).

The following compound was prepared in analogy:

| | | | | LC-MS m/z |
|---|---|---|---|---|
| Interme- | | | | [M + H]⁺, Rt |
| diate | Structure | Chemical Name | Precursors | [min] (Method) |
| INT- 158 | 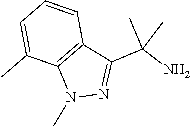 | 2-(1,7-dimethyl-1H- indazol-3-yl)propan-2- amine | INT-157 | 204, 2.44 (A) |
| INT- 162 | 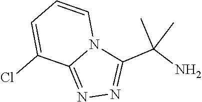 | 2-(8-chloro- [1,2,4]triazolo[4,3- a]pyridin-3-yl)propan-2- amine | INT-161 | 211, 1.53 (A) |
| INT- 166 | 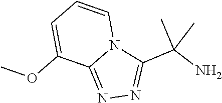 | 2-(8- methoxyimidazo[1,5- a]pyridin-3-yl)propan-2- amine | INT-165 | 306, 1.34 (P) |

### Synthesis of INT-033: 3,3-difluorocyclobutane-1-carbaldehyde

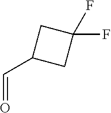

To a solution of (3,3-difluorocyclobutyl) methanol (1.0 g, 8.2 mmol) in CH₂Cl₂ (60 mL) was added DMP (3.8 g, 9.0 mmol) portion-wise and stirred at room temperature for 16 h. The reaction was quenched by addition of aq. NaHCO₃ solution (20 mL) and filtered through a pad of celite. The organic layer was separated and washed with aq. NaHCO₃ Solution (2×50 mL), aq. Na₂S₂O₃ solution (25 mL) and aq. sat. NaCl solution (25 mL). The organic layer was dried under anhyd. Na₂SO₄ and concentrated under reduced pressure to obtain the title compound (950 mg, 97%) which was used in the next step without further purification.

### Synthesis of INT-034: 1-(tert-butyl) 2-methyl (S)-4-methylpiperazine-1,2-dicarboxylate

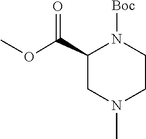 [SMILES: COC(=O)[C@@H]1CN(C)CCN1C(=O)OC(C)(C)C]

To a solution of 1-(tert-butyl) 3-methyl (S)-piperazine-1,3-dicarboxylate (2.5 g, 10.2 mmol) in CH₂Cl₂ (20 mL) was added formaldehyde (37% aq. solution, 3.0 mL, 30.7 mmol) and the mixture was stirred for 10 min room temperature. The mixture was cooled to 0° C. before NaBH(OAc)₃ (3.25 g, 15.4 mmol) was added and stirring was continued at room temperature for 16 h. The reaction was quenched by addition of aq. NH₄Cl solution (20 mL) and extracted with CH₂Cl₂ (2×50 mL). The combined organic layers were washed with aq. sat. NaCl solution (25 mL), dried over anhyd. Na₂SO₄ and concentrated under reduced pressure. The residue was purified by column chromatography (silica, CH₂Cl₂/MeOH 99:1-90:10) to obtain the title compound (2.5 g, 94%). LCMS m/z=259 [M+H]⁺, Rₜ=2.99 min (Method A).

### Synthesis of INT-035: 1-(tert-butyl) 3-methyl (S)-4-((tetrahydro-2H-pyran-4-yl)methyl)piperazine-1,3-dicarboxylate

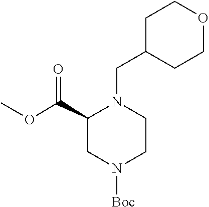 [SMILES: COC(=O)[C@@H]1CN(C(=O)OC(C)(C)C)CCN1CC1CCOCC1]

To a solution of 1-(tert-butyl) 3-methyl (S)-piperazine-1,3-dicarboxylate (500 mg, 2.1 mmol) in CH₂Cl₂ (10 mL) was added tetrahydro-2H-pyran-4-carbaldehyde (233 mg, 2.1 mmol) at room temperature and the mixture was stirred at that temperature for 10 min. NaBH(OAc)₃ (651 mg, 3.1 mmol) was added and stirring was continued for 16 h at room temperature. The mixture was poured into 5% aqueous citric acid solution (50 mL) and extracted with CH₂Cl₂ (2×50 mL). The organic layer was washed with water, sat. aq. NaCl solution, dried over Na₂SO₄ and concentrated under reduced pressure to obtain the title compound (600 mg, 85%) which was used in the next step without further purification. LCMS m/z=343 [M+H]⁺, Rₜ=1.79 min (Method C).

The following compounds were prepared in analogy:

| | | | | LC-MS m/z |
|---|---|---|---|---|
| | | | | [M + H]⁺, Rt |
| Inter- | | | | [min] |
| mediate | Structure | Chemical Name | Precursors | (Method) |
| INT- 036 | 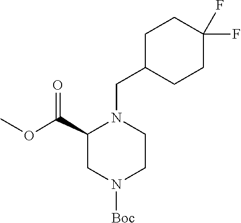 [SMILES: COC(=O)[C@@H]1CN(C(=O)OC(C)(C)C)CCN1CC1CCC(F)(F)CC1] | 1-(tert-butyl) 3-methyl (S)-4-((4,4-difluorocyclo- hexyl)methyl)piperazine- 1,3-dicarboxylate | 4,4- difluoro- cyclohexane- 1- carbaldehyde | 377, 3.76 (A) |
| INT- 037 | 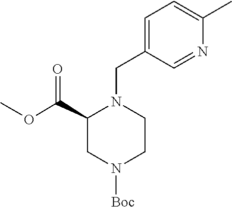 | 1-(tert-butyl) 3-methyl (S)-4-((6-methylpyridin- 3-yl)methyl)piperazine- 1,3-dicarboxylate | 6-methyl- nicotinaldehyde | 350, 3.10 (A) |
| INT- 038 | 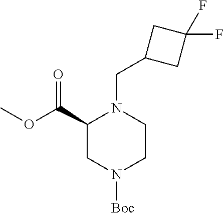 [SMILES: COC(=O)[C@@H]1CN(C(=O)OC(C)(C)C)CCN1CC1CC(F)(F)C1] | 1-(tert-butyl) 3-methyl (S)-4-((3,3-difluorocyclo- butyl)methyl)piperazine- 1,3-dicarboxylate | INT-033 | 349, 3.75 (A) |
| INT- 039 | 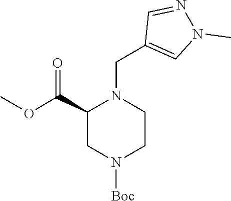 | 1-(tert-butyl) 3-methyl (S)-4-((1-methyl-1H- pyrazol-4-yl)methyl) piperazine-1,3- dicarboxylate | 1-methyl- 1H- pyrazole-4- carbaldehyde | 339, 2.91 (A) |
| INT- 040 | 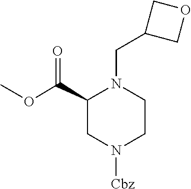 [SMILES: COC(=O)[C@@H]1CN(C(=O)OCc2ccccc2)CCN1CC1COC1] | 1-benzyl 3-methyl (S)-4- (oxetan-3-ylmethyl) piperazine-1,3- dicarboxylate | INT-045 and oxetane-3- carbaldehyde | 349, 1.70 (A) |

### Synthesis of INT-041: 1-(tert-butyl) 3-methyl (S)-4-benzylpiperazine-1,3-dicarboxylate

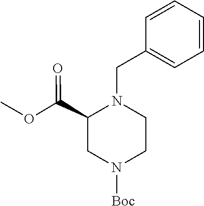 [SMILES: COC(=O)[C@@H]1CN(C(=O)OC(C)(C)C)CCN1Cc1ccccc1]

To a solution of 1-(tert-butyl) 3-methyl (S)-piperazine-1,3-dicarboxylate (250 mg, 1.02 mmol) in MeOH (10 mL) were added benzaldehyde (130 mg, 1.23 mmol) and 4 Å molecular sieves (150 mg) at room temperature and the mixture was stirred for 15 min. The mixture was cooled to 0° C. and NaBH₃CN (129 mg, 2.05 mmol) was added and stirring was continued at room temperature for 16 h. The mixture was filtered, the filter cake washed with MeOH (25 mL) and the organic solutions concentrated under reduced pressure. The residue was diluted with CH₂Cl₂ (50 mL) and washed with H₂O (50 mL), aq. sat. NaCl solution (50 mL), dried over anhyd. Na₂SO₄ and concentrated under reduced pressure. The residue was purified by column chromatography (silica, hexane/EtOAc 80:20-70:30) to obtain the title compound (150 mg, 44%). LCMS m/z=335 [M+H]⁺, Rₜ=3.81 min (Method A).

### Synthesis of INT-042: 1-(tert-butyl) 3-methyl (S)-4-((2-methylpyridin-4-yl)methyl)piperazine-1,3-dicarboxylate

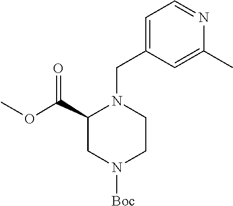

To a solution of 1-(tert-butyl) 3-methyl (S)-piperazine-1,3-dicarboxylate (500 mg, 2.04 mmol) in CH₂Cl₂/THF (4:1, 20 mL) was added 2-methylisonicotinaldehyde (372 mg, 3.07 mmol) at 0° C. and the mixture was stirred at room temperature for 2 h. The mixture was cooled to 0° C. before NaBH(OAc)₃ (869 mg, 4.09 mmol) was added. The mixture was allowed to warm to room temperature and stirring was continued for 16 h before it was concentrated, diluted with EtOAc (200 mL) and washed with H₂O (40 mL) and aq. sat. NaCl solution (40 mL). The organic layer was dried over anhyd. Na₂SO₄ and concentrated under reduced pressure. The residue was purified by column chromatography (silica gel, hexane/EtOAc 2:8) to obtain the title compound (700 mg, 98%). LCMS m/z=350 [M+H]⁺, Rₜ=3.28 min (Method A).

### Synthesis of INT-043: 1-(tert-butyl) 3-methyl (S)-4-(2-methoxyethyl)piperazine-1,3-dicarboxylate

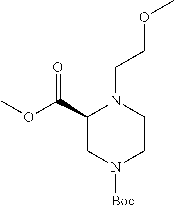

To a solution of 1-(tert-butyl) 3-methyl (S)-piperazine-1,3-dicarboxylate (400 mg, 1.63 mmol) and 1-bromo-2-methoxyethane (455 mg, 3.27 mmol) in DMF (6 mL) was added K₂CO₃ (565 mg, 4.09 mmol) at room temperature before the mixture was heated to 100° C. and stirred for 16 h at that temperature. The reaction was quenched by addition of cold H₂O (50 mL) and extracted in EtOAc (2×100 mL). The combined organic layers were washed with aq. sat. NaCl solution (50 mL), dried over anhyd, Na₂SO₄ and concentrated under reduced pressure. The residue was purified by column chromatography (silica, hexane/EtOAc 70:30-60:40) to obtain the title compound (180 mg, 36%). LCMS m/z=303 [M+H]⁺, Rₜ=1.68 min (Method A).

### Synthesis of INT-044: 1-(tert-butyl) 3-methyl (S)-4-(2,2,2-trifluoroethyl)piperazine-1,3-dicarboxylate

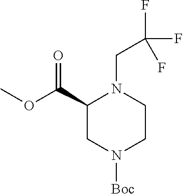 [SMILES: COC(=O)[C@@H]1CN(C(=O)OC(C)(C)C)CCN1CC(F)(F)F]

To a solution of 1-(tert-butyl) 3-methyl (S)-piperazine-1,3-dicarboxylate (0.8 g, 3.28 mmol) in MeCN (20 ml) was added K₂CO₃ (1.4 g, 9.8 mmol) at room temperature and the mixture was stirred for 15 min. 2,2,2-trifluoroethyl trifluoromethanesulfonate (2.4 ml, 16.3 mmol) was added and stirring was continued for 48 h. The mixture was extracted with EtOAc (2×10 mL) and the combined organic layers were washed with aq. sat. NaCl solution (2×10 mL), dried over anhyd. Na₂SO₄ and concentrated under reduced pressure. The residue was then purified by column chromatography (silica, hexane/EtOAc 1:0-1:1) to obtain the title compound (0.82 g, 76%). ¹H NMR (400 MHz, CDCl₃) δ 4.32 (d, 1H), 3.89 (m, 1H), 3.71 (s, 3H), 3.51 (s, 1H), 3.25-3.09 (m, 4H), 3.00 (m, 1H), 2.67 (m, 1H), 1.42 (s, 9H).

### Synthesis of INT-045: 1-benzyl 3-methyl (S)-piperazine-1,3-dicarboxylate

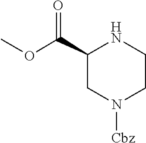 [SMILES: COC(=O)[C@@H]1CN(C(=O)OCc2ccccc2)CCN1]

To a solution of 1-(tert-butyl) 2-methyl (S)-piperazine-1,2-dicarboxylate (0.52 g, 2.13 mmol) in THF (10 mL) was added NaHCO₃ (0.54 g, 6.39 mmol) followed by benzyl carbonochloridate (0.46 ml, 3.19 mmol) and the mixture was stirred at room temperature for 16 h. Pyridine (3 drops) was added to the mixture before diluting with EtOAc (50 ml). The mixture was washed with H₂O (50 ml) and aq. sat. NaCl solution (30 ml), dried over anhyd. Na₂SO₄ and concentrated under reduced pressure to obtain the corresponding Cbz-protected amine (0.8μ) which was used in the next step without further purification.

To a solution of above carbamate (0.8 g, 2.11 mmol) in CH₂Cl₂ (10 ml) was added TFA (1 ml) at 0° C. and the mixture was allowed to warm to room temperature and stirred for 1 h before being concentrated under reduced pressure to obtain the title compound (0.5 g, 85% over two steps) as its TFA salt which was used in the next step without further purification. LCMS m/z=279 [M+H]⁺, Rₜ=2.80 min (Method A).

### Synthesis of INT-046: (S)-4-(tert-butoxycarbonyl)-1-methylpiperazine-2-carboxylic acid

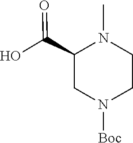 [SMILES: CN1CCN(C(=O)OC(C)(C)C)C[C@H]1C(=O)O]

A suspension of (S)-4-(tert-butoxycarbonyl)piperazine-2-carboxylic acid (50 g, 217 mmol) in MeOH (1.2 L) was purged with nitrogen for 10 minutes. To the mixture were added formaldehyde (37% aq. solution, 44 mL, 542 mmol) and 10%-Pd/C (25μ) and the mixture was set under hydrogen gas (2 bar) in a Parr shaker and stirred at room temperature for 16 h. The mixture was filtered through a pad of celite and the pad was washed with CH₂Cl₂/MeOH (9:1). The filtrate was concentrated under reduced pressure and the residue was triturate with hexane, filtered and dried under reduced pressure to afford the title compound (45 g, 84%). LCMS m/z=245 [M+H]⁺, Rₜ=1.74 min (Method A).

### Synthesis of INT-047: (S)-1-(tert-butoxycarbonyl)-4-methylpiperazine-2-carboxylic acid

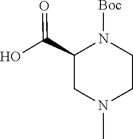 [SMILES: CN1CCN(C(=O)OC(C)(C)C)[C@H](C(=O)O)C1]

To a solution of ester INT-034 (2.74 g, 10.6 mmol) in THF/MeOH/H₂O (4:2:1, 66 mL) was added LiOH·H₂O (0.67 g, 15.9 mmol) at room temperature and the mixture was stirred 16 h. The mixture was concentrated under reduced pressure and dried by azeotrope water removal with PhMe (2×3 mL) under reduced pressure to obtain the title compound (2.6 g, 97%) as its lithium carboxylate salt which was used in the next step without further purification. LCMS m/z=245 [M+H]⁺, Rₜ=1.48 min (Method A).

In some cases, the lithium carboxylate was converted to the free carboxylic acid as follows: The product was dissolved in water and acidified to pH=5 with aq. NaHSO₄ solution and the mixture was extracted with EtOAc. The combined organic layers were washed with aq. sat. NaCl solution, dried over anhyd. Na₂SO₄ and concentrated under reduced pressure to obtain the corresponding carboxylic acid.

The following compounds were prepared in analogy:

| | | | | LC-MS m/z |
|---|---|---|---|---|
| | | | | [M + H]⁺, Rt |
| Inter- | | | | [min] |
| mediate | Structure | Chemical Name | Precursors | (Method) |
| INT- 048 | 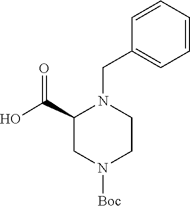 [SMILES: CC(C)(C)OC(=O)N1CCN(Cc2ccccc2)[C@H](C(=O)O)C1] | (S)-1-benzyl-4-(tert- butoxycarbonyl) piperazine- 2-carboxylic acid | INT-041 | 321, 2.44 (A) |
| INT- 049 | 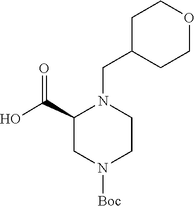 [SMILES: CC(C)(C)OC(=O)N1CCN(CC2CCOCC2)[C@H](C(=O)O)C1] | (S)-4-(tert- butoxycarbonyl)-1- ((tetrahydro- 2H-pyran-4-yl) methyl)piperazine-2- carboxylic acid | INT-035 | 329, 1.46 (C) |
| INT- 050 | 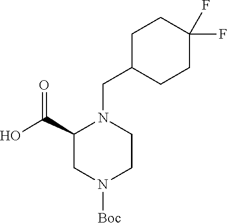 [SMILES: CC(C)(C)OC(=O)N1CCN(CC2CCC(F)(F)CC2)[C@H](C(=O)O)C1] | (S)-4-(tert- butoxycarbonyl)- 1-((4,4-difluoro- cyclohexyl)methyl) piperazine-2-carboxylic acid | INT-036 | 361 [M − H]⁻, 2.64 (A) |
| INT- 051 | 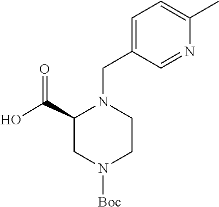 [SMILES: Cc1ccc(CN2CCN(C(=O)OC(C)(C)C)C[C@H]2C(=O)O)cn1] | (S)-4-(tert- butoxycarbonyl)- 1-((6-methylpyridin- 3-yl)methyl)piperazine- 2-carboxylic acid | INT-037 | 336, 2.08 (A) |
| INT- 052 | 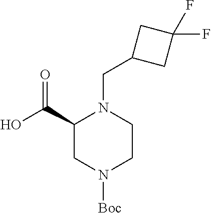 [SMILES: CC(C)(C)OC(=O)N1CCN(CC2CC(F)(F)C2)[C@H](C(=O)O)C1] | (S)-4-(tert- butoxycarbonyl)- 1-((3,3-difluoro- cyclobutyl)methyl) piperazine-2-carboxylic acid | INT-038 | 333 [M − H]⁻, 2.43 (A) |
| INT- 053 | 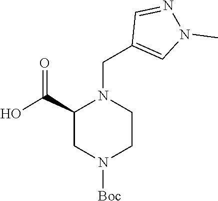 [SMILES: Cn1cc(CN2CCN(C(=O)OC(C)(C)C)C[C@H]2C(=O)O)cn1] | (S)-4-(tert- butoxycarbonyl)- 1-((1-methyl-1H- pyrazol-4-yl)methyl) piperazine-2-carboxylic acid | INT-039 | 325, 1.82 (A) |
| INT- 054 | 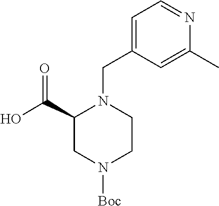 [SMILES: Cc1cc(CN2CCN(C(=O)OC(C)(C)C)C[C@H]2C(=O)O)ccn1] | (S)-4-(tert- butoxycarbonyl)- 1-((2-methylpyridin- 4-yl)methyl) piperazine- 2-carboxylic acid | INT-042 | 336, 2.06 (A) |
| INT- 055 | 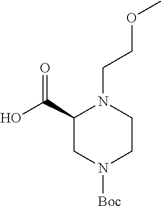 [SMILES: COCCN1CCN(C(=O)OC(C)(C)C)C[C@H]1C(=O)O] | (S)-4-(tert- butoxycarbonyl)- 1-(2-methoxy- ethyl)piperazine-2- carboxylic acid | INT-043 | 289, 1.24 (A) |
| INT- 056 | 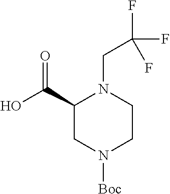 [SMILES: CC(C)(C)OC(=O)N1CCN(CC(F)(F)F)[C@H](C(=O)O)C1] | (S)-4-(tert- butoxycarbonyl)- 1-(2,2,2-trifluoro- ethyl)piperazine-2- carboxylic acid | INT-044 | 311 [M − H]⁻, 2.52 (A) |
| INT- 057 | 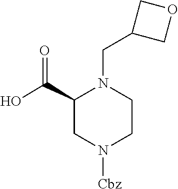 [SMILES: O=C(O)[C@@H]1CN(C(=O)OCc2ccccc2)CCN1CC1COC1] | (S)-4-((benzyloxy) carbonyl)-1-(oxetan-3- ylmethyl)piperazine-2- carboxylic acid | INT-040 | 335, 1.15 (B) |

### Synthesis of INT-145: (S)-4-(tert-butoxycarbonyl)-1-(2,2-difluoroethyl)piperazine-2-carboxylic acid

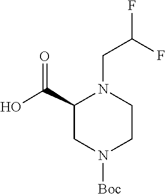 [SMILES: CC(C)(C)OC(=O)N1CCN(CC(F)F)[C@H](C(=O)O)C1]

To a solution of (S)-4-(tert-butoxy carbonyl) piperazine-2-carboxylic acid (0.25 g, 1.1 mmol) in 1,4-dioxane (5 mL) and water (5 mL) were added 2,2-difluoroethyl trifluoromethanesulfonate (0.35 g, 1.6 mmol, 1.5) and K₂CO₃ (0.31 g, 2.2 mmol) at room temperature and the mixture was stirred at 70° C. for 16 h in a sealed tube. Subsequently, the reaction mixture was diluted with water (10 ml) and washed with MTBE. The aqueous layer was acidified with NaHSO₄ solution to pH ˜4 and extracted with ethyl acetate (2×50 mL). The combined organic layers were washed with sat. aq. NaCl solution, dried over Na₂SO₄ and concentrated under reduced pressure to yield the title compound which was used in next step without further purification. LCMS m/z=295 [M+H]⁺, Rₜ=2.82 min (Method C).

### Synthesis of INT-058: rac-tert-butyl 3-((2-(8-methylimidazo[1,5-a]pyridin-3-yl)propan-2-yl)carbamoyl)piperazine-1-carboxylate

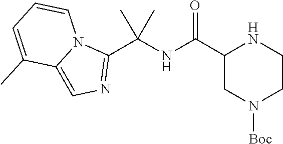 [SMILES: Cc1cccn2c(C(C)(C)NC(=O)C3CN(C(=O)OC(C)(C)C)CCN3)ncc12]

The title compound was prepared in analogy to its enantiopure counterpart INT-068 from racemic 4-(tert-butoxycarbonyl)piperazine-2-carboxylic acid as the starting material.

### Synthesis of INT-059: (S)-1-(((9H-fluoren-9-yl)methoxy)carbonyl)-4-(tert-butoxycarbonyl)piperazine-2-carboxylic acid

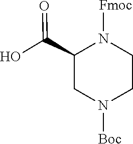

A mixture of (S)-4-(tert-butoxycarbonyl)piperazine-2-carboxylic acid (30 g, 130 mmol) in aq. 10% NaHCO₃ solution (500 mL) was stirred for 10 min at room temperature before a solution of (9H-fluoren-9-yl)methyl (2,5-dioxopyrrolidin-1-yl) carbonate (52.7 g, 156 mmol) in 1,4-dioxane (150 mL) was added and stirring was continued for 16.5 h. The mixture was diluted with H₂O (700 mL) and its pH adjusted to pH=3 with aq. HCl solution (3 M). The mixture was extracted with EtOAc (600 mL) and the organic layer was washed with aq. sat. NaCl solution (300 mL), dried over Na₂SO₄ and concentrated under reduced pressure. The residue was triturated with PE (500 mL) at room temperature for 1 h. The mixture was filtered to obtain the title compound (60.0 g, 99%). LCMS m/z=475 [M+Na]⁺; ¹H NMR (400 MHz, DMSO-d₆) δ 13.30-12.93 (m, 1H), 8.00-7.85 (m, 2H), 7.71-7.58 (m, 2H), 7.48-7.40 (m, 2H), 7.39-7.26 (m, 2H), 4.67-4.53 (m, 1H), 4.44-4.19 (m, 4H), 3.99-3.84 (m, 1H), 3.83-3.68 (m, 1H), 3.27-2.99 (m, 2H), 2.95-2.71 (m, 1H), 1.48-1.32 (m, 9H).

### Synthesis of INT-060: (1-(trifluoromethyl)cyclopropyl)methyl trifluoromethanesulfonate

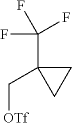 [SMILES: O=S(=O)(OCC1(C(F)(F)F)CC1)C(F)(F)F]

To a solution of 1-(trifluoromethyl)cyclopropane-1-carboxylic acid (300 mg, 1.94 mmol) in Et₂O (5 mL) was added LiAlH₄ (2.4 M in THF, 1.2 mL, 2.92 mmol) at 0° C. and the mixture was stirred at room temperature for 16 h. The reaction was quenched by addition of aq. HCl solution (1N, 5 mL) at 0° C. and extracted with Et₂O (2×30 mL). The combined organic layers were washed with aq. sat. NaCl solution, dried over anhyd. Na₂SO₄ and concentrated under reduced pressure to obtain the corresponding primary alcohol (260 mg, 95%) which was used in the next step without further purification.

To a solution of above alcohol (250 mg, 1.78 mmol) and 2,6-lutidine (0.3 mL, 2.32 mmol) in CH₂Cl₂ (5 mL) was added trifluoromethanesulfonic anhydride (0.4 mL, 2.32 mmol) at 0° C. and the mixture stirred for 2 h. The mixture was washed with aq. HCl solution (1M, 2×10 mL) and aq. sat. NaHCO₃ solution (2×10 mL). The aqueous layer was extracted with CH₂Cl₂ and the combined organic layers were dried over anhyd. Na₂SO₄ and concentrated under reduced pressure to obtain the title compound (350 mg, 72%) which was used without further purification in the next step. ¹H NMR (400 MHz, DMSO-d₆) (3.54 (s, 2H), 0.87-0.89 (m, 4H).

### Synthesis of INT-061: 3,3-difluorocyclobutyl trifluoromethanesulfonate

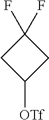 [SMILES: O=S(=O)(OC1CC(F)(F)C1)C(F)(F)F]

To a solution of 3,3-difluorocyclobutan-1-ol (100 mg, 0.92 mmol) and 2,6-lutidine (0.14 mL, 1.2 mmol) in CH₂Cl₂ (5 mL) was added trifluoromethanesulfonic anhydride (0.2 mL, 1.2 mmol) at 0° C. and the mixture stirred for 2 h. The mixture was washed with aq. HCl solution (1M, 2×20 mL) and aq. sat. NaHCO₃ solution (2×20 mL). The aqueous layer was extracted with CH₂Cl₂ (50 mL) and the combined organic layers were dried over anhyd. Na₂SO₄ and concentrated under reduced pressure to obtain the title compound (120 mg, 41%) which was used without further purification in the next step. ¹H NMR (400 MHz, DMSO-d₆) (5.02 (m, 1H), 3.13 (m, 2H), 2.95 (m, 2H).

### Synthesis of INT-062: methyl 2-cyclopropylisonicotinate

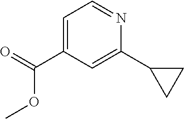

To a degassed solution of methyl 2-chloroisonicotinate (1.0 g, 5.81 mmol) in 1,4-dioxane (20 mL) were added cyclopropylboronic acid (750 mg, 8.72 mmol), Ag₂O (1.3 g, 5.81 mmol), K₃PO₄ (3.6 g, 17.44 mmol) and Pd(dppf)Cl₂ (426 mg, 0.58 mmol) at room temperature under nitrogen atmosphere and heated to 100° C. for 16 h. Reaction mixture was filtered, diluted with EtOAc (300 mL), washed with H₂O (80 mL) and aq. sat. NaCl solution (80 mL). The organic layer was dried over anhyd. Na₂SO₄, filtered and concentrated under reduced pressure. The residue was purified by column chromatography (silica gel, hexane/EtOAc 90:10-88:12) to obtain the title compound (700 mg, 67%). LCMS m/z=178 [M+H]⁺, Rₜ=3.16 min (Method A).

### Synthesis of INT-063: (2-cyclopropylpyridin-4-yl)methanol

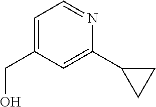 [SMILES: OCc1ccnc(C2CC2)c1]

To a solution of methyl ester INT-062 (1.1 g, 6.21 mmol) in MeOH (15 mL) was added NaBH₄ (708 mg, 18.6 mmol) at 0° C. and the mixture was stirred at room temperature for 24 h. The mixture was filtered, diluted with EtOAc (300 mL), washed with H₂O (100 mL) and aq. sat. NaCl solution (100 mL). The organic layer was dried over Na₂SO₄, concentrated under reduced pressure and the residue was purified by column chromatography (silica gel, hexane/EtOAc 70:30) to obtain the title compound (700 mg, 76%). LCMS m/z=150 [M+H]⁺, Rₜ=1.92 min (Method A).

### Synthesis of INT-064: (2-cyclopropylpyridin-4-yl)methyl methanesulfonate

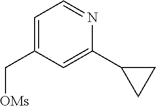 [SMILES: CS(=O)(=O)OCc1ccnc(C2CC2)c1]

To a solution of alcohol INT-063 (300 mg, 2.01 mmol) in CH₂Cl₂ (15 mL) was added Et₃N (0.8 mL, 6.04 mmol) at 0° C. followed by the addition of MsCl (0.2 mL, 3.02 mmol) and the mixture was stirred at room temperature for 30 min. The mixture was diluted with CH₂Cl₂ (200 mL), washed with H₂O (50 mL), aq. sat. NaCl solution (50 mL) and dried over Na₂SO₄. The mixture was concentrated under reduced pressure to obtain the title compound (400 mg, 66%) which was used without further purification in the next step. LCMS m/z=228 [M+H]⁺, Rₜ=1.51 min (Method H).

### Synthesis of INT-065: methyl 4,4-difluoro-1-methylcyclohexane-1-carboxylate

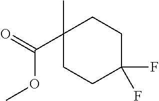

To a solution of methyl 4,4-difluorocyclohexane-1-carboxylate (2.0 g, 11.2 mmol) in THF (50 mL) was added LDA (2 M in THF, 8.5 mL, 16.9 mmol) at −78° C. and the mixture was stirred for 1 h. Methyl iodide (0.84 mL, 13.48 mmol) was added and the mixture was allowed to warm to room temperature and stirred for 16 h. The mixture was quenched by addition of aq. NH₄Cl solution (50 mL) and extracted with EtOAc (100 mL). The organic layer was washed with aq. sat. NaCl solution (50 mL), dried over anhyd. Na₂SO₄ and concentrated under reduced. The residue was purified by column chromatography (silica, hexane/EtOAc 80:20) to obtain the title compound (1.2 g, 56%). ¹H NMR (400 MHz, CDCl₃) δ 3.70 (s, 3H), 2.17 (d, 2H), 1.95 (m, 2H), 1.87-1.73 (m, 2H), 1.50 (m, 2H), 1.21 (2, 3H).

### Synthesis of INT-066: (4,4-difluoro-1-methylcyclohexyl)methanol

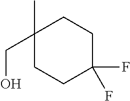

To a solution ester INT-065 (500 mg, 2.60 mmol) in THF (10 mL) was added LiAlH₄ (2.4 M in THF, 2.2 mL, 1.0 mmol) at 0° C. and the mixture was allowed to warm to room temperature and stirred for 16 h. The reaction was quenched by addition of a H₂O/THF (1:9) and the mixture was filtered through a pad of celite. The filtrate was concentrated under reduced pressure to obtain the title compound (400 mg, 94%). ¹H NMR (400 MHz, DMSO-d₆) δ 4.57 (t, 1H), 3.16 (d, 2H), 1.90-1.82 (m, 4H), 1.54-1.43 (m, 2H), 1.30-1.17 (m, 2H), 0.86 (s, 3H).

### Synthesis of INT-067: (4,4-difluoro-1-methylcyclohexyl)methyl trifluoromethanesulfonate

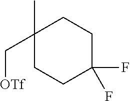 [SMILES: CC1(COS(=O)(=O)C(F)(F)F)CCC(F)(F)CC1]

To a solution of alcohol INT-066 (200 mg, 1.22 mmol) in CH₂Cl₂ (10 mL) were added 2,6-lutidine (0.7 mL, 6.09 mmol) and triflic anhydride (0.26 mL, 1.58 mmol) at 0° C. and the mixture was allowed to warm to room temperature and stirred for 3 h. The mixture was diluted with H₂O (20 mL) and extracted with EtOAc (30 mL), washed with aq. sat. NaCl solution, dried over anhyd. Na₂SO₄ and concentrated under reduced pressure to obtain the title compound (200 mg, 55%) which was used in the next step without further purification. ¹H NMR (400 MHz, CDCl₃) δ 4.25 (s, 2H), 1.98-1.88 (m, 4H), 1.66-1.55 (m, 4H), 1.10 (s, 3H).

### Synthesis of INT-068: tert-butyl (S)-3-((2-(8-methylimidazo[1,5-a]pyridin-3-yl)propan-2-yl)carbamoyl)piperazine-1-carboxylate

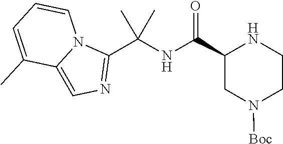 [SMILES: Cc1cccn2c(C(C)(C)NC(=O)[C@@H]3CN(C(=O)OC(C)(C)C)CCN3)ncc12]

To a solution of piperazine INT-059 (45.5 g, 100 mmol) in DMF (220 mL) were added HATU (36.4 g, 95.8 mmol) and DIPEA (32.5 g, 251 mmol) at room temperature and the mixture was stirred for 1 h under nitrogen atmosphere before amine INT-004 (22.0 g, 83.9 mmol) was added and stirring was continued for 16 h. H₂O (1 L) was added, the mixture was filtered and the filter cake washed with H₂O (100 mL). The filter cake was dissolved in CH₂Cl₂ (1 L) and washed with H₂O (3×50 mL), dried over anhyd. Na₂SO₄ and concentrated under reduced pressure. The crude carboxamide residue (69μ) was used in the next step without further purification. LCMS m/z=624 [M+H]⁺.

To a solution of above carboxamide (69 g, 110 mmol) in CH₂Cl₂ (690 mL) was added piperidine (10.5 g, 121 mmol) at room temperature under nitrogen atmosphere and the mixture was stirred for 16 h before it was filtered and concentrated under reduced pressure. The residue was purified by column chromatography (silica, PE/EtOAc 50:1-30:1) to obtain the title compound (31.3 g, 78% over two steps). LCMS m/z=402 [M+H]⁺; ¹H NMR (400 MHz, DMSO-d₆) δ 8.27 (s, 1H), 8.18-8.09 (m, 1H), 7.26 (s, 1H), 6.51 (d, 2H), 3.84 (s, 1H), 3.63 (d, 1H), 3.21-3.11 (m, 1H), 3.07-2.97 (m, 1H), 2.86 (d, 1H), 2.75 (d, 1H), 2.71-2.60 (m, 1H), 2.35 (s, 3H), 1.70 (d, 6H), 1.40 (s, 9H).

### Synthesis of INT-069: tert-butyl (S)-4-methyl-3-((2-(8-methylimidazo[1,5-a]pyridin-3-yl)propan-2-yl)carbamoyl)piperazine-1-carboxylate

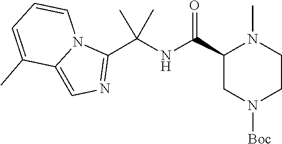 [SMILES: Cc1cccn2c(C(C)(C)NC(=O)[C@@H]3CN(C(=O)OC(C)(C)C)CCN3C)ncc12]

To a solution of carboxamide INT-068 (300 mg, 0.75 mmol) in MeOH (10 mL) were added formaldehyde (37% aq., 0.11 mL, 1.12 mmol) and AcOH (0.5 mL) at room temperature and the mixture was stirred for 2 h before it was cooled to 0° C. and NaBH₃CN (141 mg, 2.24 mmol) was added. The mixture was allowed to warm to room temperature and stirring was continued for 16 h. The reaction was quenched by addition of aq. NH₄Cl (10 mL) and the mixture was extracted with CH₂Cl₂ (2×25 mL). The combined organic layers were washed with aq. sat. NaCl solution (15 mL), dried over anhyd. Na₂SO₄ and concentrated under reduced pressure. The residue was purified by column chromatography (silica, CH₂Cl₂/MeOH 9:1) to obtain the title compound (250 mg, 81%). LCMS m/z=416 [M+H]⁺, Rₜ=2.98 min (Method A)

### Synthesis of INT-070: tert-butyl (S)-3-((2-(8-methylimidazo[1,5-a]pyridin-3-yl)propan-2-yl)carbamoyl)-4-(2,2,2-trifluoroethyl)piperazine-1-carboxylate

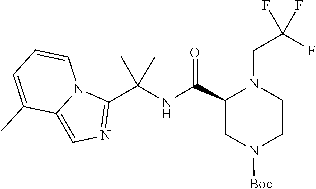 [SMILES: Cc1cccn2c(C(C)(C)NC(=O)[C@@H]3CN(C(=O)OC(C)(C)C)CCN3CC(F)(F)F)ncc12]

To a solution of carboxamide INT-068 (200 mg, 0.49 mmol) in THF (5 mL) were added DIPEA (0.08 mL, 0.49 mmol) followed by 2,2,2-trifluoroethyl trifluoromethanesulfonate (0.07 mL, 0.49 mmol) at room temperature. The mixture was stirred at 80° C. for 4 h before it was allowed to cool to room temperature and concentrated under reduced pressure. The residue was purified by column chromatography (silica, CH₂Cl₂/MeOH 9:1) to obtain the title compound (200 mg, 84%). LCMS m/z=484 [M+H]⁺, Rt=3.45 min (Method A).

The following compounds were prepared in analogy:

| | | | | LC-MS |
|---|---|---|---|---|
| | | | | m/z |
| | | | | [M + H]⁺, |
| Inter- | | | | Rt [min] |
| mediate | Structure | Chemical Name | Precursors | (Method) |
| INT- 071 | 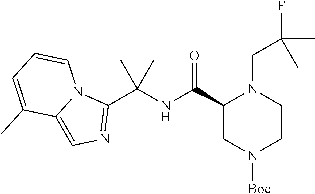 [SMILES: Cc1cccn2c(C(C)(C)NC(=O)[C@@H]3CN(C(=O)OC(C)(C)C)CCN3CC(C)(C)F)ncc12] | tert-butyl (S)-4-(2- fluoro-2- methylpropyl)- 3-((2-(8- methylimidazo[1,5- a]pyridin-3-yl) propan-2-yl) carbamoyl) piperazine-1- carboxylate | INT-068 and 2-fluoro-2- methylpropyl trifluoro- methane- sulfonate | 476, 1.91 (A) |
| INT- 072 | 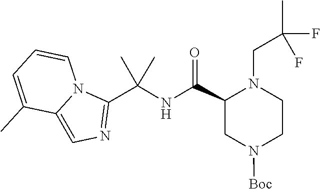 [SMILES: Cc1cccn2c(C(C)(C)NC(=O)[C@@H]3CN(C(=O)OC(C)(C)C)CCN3CC(C)(F)F)ncc12] | tert-butyl (S)-4-(2,2- difluoropropyl)-3- ((2-(8-methylimidazo [1,5-a]pyridin-3- yl)propan-2-yl) carbamoyl) piperazine-1- carboxylate | INT-068 and 2,2-difluoro- propyl trifluoro- methane- sulfonate | 480, 3.35 (A) |
| INT- 073 | 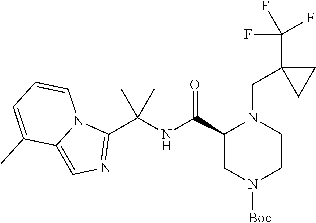 [SMILES: Cc1cccn2c(C(C)(C)NC(=O)[C@@H]3CN(C(=O)OC(C)(C)C)CCN3CC3(C(F)(F)F)CC3)ncc12] | tert-butyl (S)-3-((2- (8-methylimidazo- [1,5-a]pyridin-3- yl)propan-2-yl) carbamoyl)-4-((1- (trifluoromethyl) cyclopropyl)methyl) piperazine-1- carboxylate | INT-068 and INT-060 | 524, 1.75 (A) |
| INT- 074 | 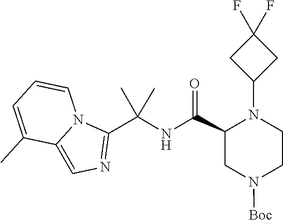 [SMILES: Cc1cccn2c(C(C)(C)NC(=O)[C@@H]3CN(C(=O)OC(C)(C)C)CCN3C3CC(F)(F)C3)ncc12] | tert-butyl (S)-4-(3,3- difluorocyclobutyl)- 3-((2-(8- methylimidazo [1,5-a]pyridin-3- yl)propan-2-yl) carbamoyl) piperazine-1- carboxylate | INT-068 and INT-061 | 492, 3.34 (A) |

### Synthesis of INT-075: tert-butyl (S)-4-((2-cyclopropylpyridin-4-yl)methyl)-3-((2-(8-methylimidazo[1,5-a]pyridin-3-yl)propan-2-yl)carbamoyl)piperazine-1-carboxylate

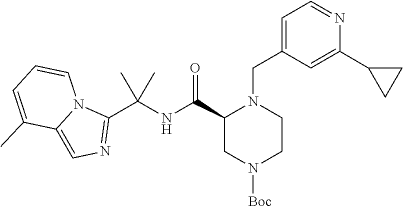 [SMILES: Cc1cccn2c(C(C)(C)NC(=O)[C@@H]3CN(C(=O)OC(C)(C)C)CCN3Cc3ccnc(C4CC4)c3)ncc12]

To a solution of carboxamide INT-068 (150 mg, 0.37 mmol) in DMF (5 mL) were added K₂CO₃ (103 mg, 0.74 mmol) followed by methansulfonate INT-064 (170 mg, 0.74 mmol) at room temperature. The mixture was stirred at 80° C. for 16 h before it was allowed to cool to room temperature, filtered and diluted with EtOAc (150 mL). The mixture was washed with H₂O (2×30 mL) and aq. sat. NaCl solution (50 mL). The organic layer was dried over anhyd. Na₂SO₄ and concentrated under reduced pressure. The residue was purified by column chromatography (silica, CH₂Cl₂/MeOH 8:2) to obtain the title compound (150 mg, 76%). LCMS m/z=533 [M+H]⁺, Rₜ=3.38 min (Method A).

### Synthesis of INT-076: tert-butyl (S)-4-(2-hydroxy-2-methylpropyl)-3-((2-(8-methylimidazo[1,5-a]pyridin-3-yl)propan-2-yl)carbamoyl)piperazine-1-carboxylate

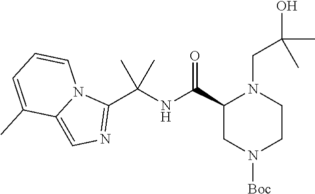 [SMILES: Cc1cccn2c(C(C)(C)NC(=O)[C@@H]3CN(C(=O)OC(C)(C)C)CCN3CC(C)(C)O)ncc12]

To a solution of carboxamide INT-068 (500 mg, 1.25 mmol) in MeOH (8 mL) and THF (1 mL) was added 2,2-dimethyloxirane (1.0 g, 14.9 mmol) at room temperature and the mixture was stirred at 40° C. for 72 h. The mixture was concentrated under reduced pressure to yield the title compound (550 mg, 93%) which was used in the next step without further purification. LCMS m/z=474 [M+H]⁺, Rₜ=3.14 min (Method A).

### Synthesis of INT-077: tert-butyl (S)-4-((4,4-difluoro-1-methylcyclohexyl)methyl)-3-((2-(8-methylimidazo[1,5-a]pyridin-3-yl)propan-2-yl)carbamoyl)piperazine-1-carboxylate

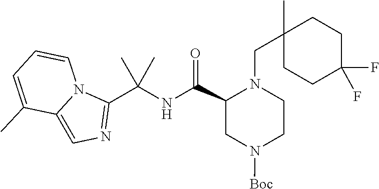 [SMILES: Cc1cccn2c(C(C)(C)NC(=O)[C@@H]3CN(C(=O)OC(C)(C)C)CCN3CC3(C)CCC(F)(F)CC3)ncc12]

To a solution of trifluormethansulfonate INT-067 (100 mg, 0.24 mmol) in MeCN (5 mL) were added DIPEA (0.4 mL, 0.74 mmol) and carboxamide INT-068 (110 mg, 0.37 mmol) at room temperature and the mixture was stirred at reflux for 16 h before it was allowed to cool to room temperature. The mixture was concentrated under reduced pressure, diluted with H₂O (30 mL) and extracted with EtOAc (50 mL). The organic layer was washed with aq. sat. NaCl solution (30 mL), dried over anhyd. Na₂SO₄ and concentrated under reduced pressure. The residue was purified by column chromatography (silica, CH₂Cl₂/MeOH 99:1) to obtain the title compound (75 mg, 57%) which was used in the next step without further purification. LCMS m/z=549 [M+H]⁺, Rₜ=2.14 min (Method G).

### Synthesis of INT-078: tert-butyl (S)-3-((2-(8-methylimidazo[1,5-a]pyridin-3-yl)propan-2-yl)carbamoyl)-4-(3-(trifluoromethyl)benzyl)piperazine-1-carboxylate

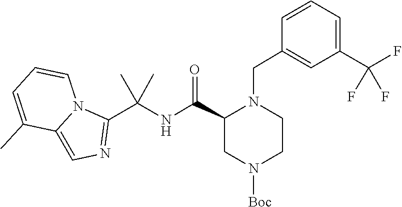 [SMILES: Cc1cccn2c(C(C)(C)NC(=O)[C@@H]3CN(C(=O)OC(C)(C)C)CCN3Cc3cccc(C(F)(F)F)c3)ncc12]

To a solution of carboxamide INT-068 (150 mg, 0.37 mmol) in CH₂Cl₂ (10 mL) was added 3-(trifluoromethyl)benzaldehyde (0.094 mL, 0.56 mmol) at room temperature and the mixture was stirred for 20 min before NaBH(OAc)₃ (119 mg, 0.56 mmol) was added at 0° C. The mixture was allowed to warm to room temperature and stirring was continued for 16 h. The reaction was quenched by addition of aq. NaHCO₃ solution (30 mL) and the mixture was extracted with CH₂Cl₂ (2×100 mL). The combined organic layers were washed with aq. sat. NaCl solution (50 mL), dried over Na₂SO₄ and concentrated under reduced pressure. The residue was purified by column chromatography (silica, hexane/EtOAc 1:3-1:9) to obtain the title compound (170 mg, 82%). LCMS m/z=560 [M+H]⁺, Rₜ=3.66 min (Method A).

The following compounds were prepared in analogy:

| | | | | LC-MS |
|---|---|---|---|---|
| | | | | m/z |
| | | | | [M + H]⁺, |
| Inter- | | | | Rt [min] |
| mediate | Structure | Chemical Name | Precursors | (Method) |
| INT- 079 | 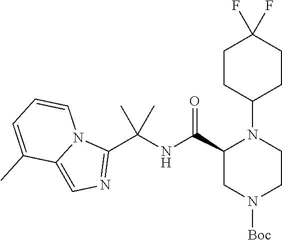 [SMILES: Cc1cccn2c(C(C)(C)NC(=O)[C@@H]3CN(C(=O)OC(C)(C)C)CCN3C3CCC(F)(F)CC3)ncc12] | tert-butyl (S)-4-(4,4- difluorocyclohexyl)-3-((2- (8-methylimidazo[1,5- a]pyridin-3-yl)propan-2- yl)carbamoyl)piperazine- 1-carboxylate | INT-068 and 4,4- difluoro- cyclohexan-1- one | 520, 1.97 (U) |
| INT- 080 | 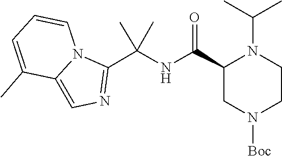 [SMILES: Cc1cccn2c(C(C)(C)NC(=O)[C@@H]3CN(C(=O)OC(C)(C)C)CCN3C(C)C)ncc12] | tert-butyl (S)-4-isopropyl- 3-((2-(8- methylimidazo [1,5-a]pyridin-3- yl)propan-2-yl) carbamoyl) piperazine-1- carboxylate | INT-068 and propan- 2-one | 444, 1.84 (U) |
| INT- 081 | 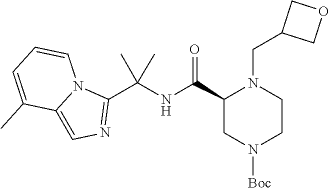 [SMILES: Cc1cccn2c(C(C)(C)NC(=O)[C@@H]3CN(C(=O)OC(C)(C)C)CCN3CC3COC3)ncc12] | tert-butyl (S)-3-((2-(8- methylimidazo[1,5-a] pyridin-3-yl) propan-2-yl) carbamoyl)-4-(oxetan-3- ylmethyl)piperazine-1- carboxylate | INT-068 and oxetane-3- carbaldehyde | 472, 2.93 (A) |

### Synthesis of INT-082: tert-butyl (S)-4-(2-fluorobenzyl)-3-((2-(8-methylimidazo[1,5-a]pyridin-3-yl)propan-2-yl)carbamoyl)piperazine-1-carboxylate

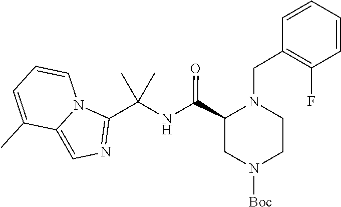 [SMILES: Cc1cccn2c(C(C)(C)NC(=O)[C@@H]3CN(C(=O)OC(C)(C)C)CCN3Cc3ccccc3F)ncc12]

To a solution of carboxamide INT-068 (150 mg, 0.37 mmol) in MeOH (10 mL) were added 2-fluorobenzaldehyde (55 mg, 0.44 mmol), AcOH (0.1 mL) and 4 Å molecular sieves (150 mg) at room temperature and the mixture was stirred for 30 min before it was cooled to 0° C. and NaBH₃CN (47 mg, 0.74 mmol) was added. The mixture was allowed to warm to room temperature and stirring was continued for 16 before it was filtered and the filter cake washed with MeOH (15 mL). The mixture was concentrated under reduced pressure and the residue diluted with CH₂Cl₂ (30 mL) and washed with H₂O (20 mL) and aq. sat. NaCl solution (20 mL). The organic layer was dried over anhyd. Na₂SO₄ and concentrated under reduced pressure. The residue was purified by column chromatography (silica, CH₂Cl₂/MeOH 95:5) to obtain the title compound (120 mg, 76%). LCMS m/z=510 [M+H]⁺, Rₜ=3.51 min (Method A).

The following compounds were prepared in analogy:

| | | | | LC-MS |
|---|---|---|---|---|
| | | | | m/z |
| | | | | [M + H]⁺, |
| Inter- | | | | Rt [min] |
| mediate | Structure | Chemical Name | Precursors | (Method) |
| INT- 083 | 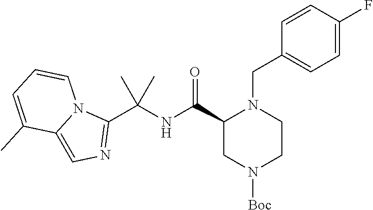 [SMILES: Cc1cccn2c(C(C)(C)NC(=O)[C@@H]3CN(C(=O)OC(C)(C)C)CCN3Cc3ccc(F)cc3)ncc12] | tert-butyl (S)-4-(4- fluorobenzyl)-3-((2- (8-methylimidazo [1,5-a]pyridin-3- yl)propan-2-yl) carbamoyl) piperazine-1- carboxylate | INT-068 and 2- fluoro- benzalde- hyde | 510, 3.60 (A) |
| INT- 084 | 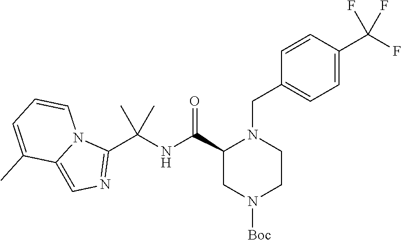 [SMILES: Cc1cccn2c(C(C)(C)NC(=O)[C@@H]3CN(C(=O)OC(C)(C)C)CCN3Cc3ccc(C(F)(F)F)cc3)ncc12] | tert-butyl (S)-3-((2- (8-methylimidazo [1,5-a]pyridin-3- yl)propan-2-yl) carbamoyl)-4- (4-(trifluoromethyl) benzyl)piperazine- 1-carboxylate | INT-068 and 4-(tri- fluoro- methyl) benzalde- hyde | 560, 3.69 (A) |
| INT- 085 | 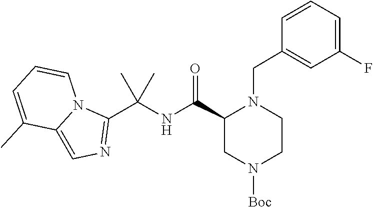 [SMILES: Cc1cccn2c(C(C)(C)NC(=O)[C@@H]3CN(C(=O)OC(C)(C)C)CCN3Cc3cccc(F)c3)ncc12] | tert-butyl (S)-4-(3- fluorobenzyl)-3-((2- (8-methylimidazo [1,5-a]pyridin-3- yl)propan-2-yl) carbamoyl) piperazine-1- carboxylate | INT-068 and 3- fluoro- benzalde- hyde | 510, 3.63 (A) |
| INT- 086 | 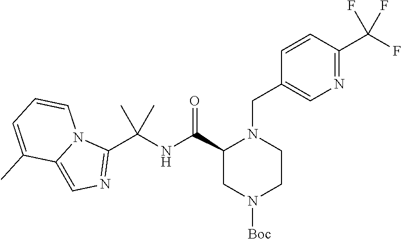 [SMILES: Cc1cccn2c(C(C)(C)NC(=O)[C@@H]3CN(C(=O)OC(C)(C)C)CCN3Cc3ccc(C(F)(F)F)nc3)ncc12] | tert-butyl (S)-3-((2- (8-methylimidazo [1,5-a]pyridin-3- yl)propan-2-yl) carbamoyl)-4-((6- (trifluoromethyl) pyridin- 3-yl)methyl) piperazine- 1-carboxylate | INT-068 and 6- (trifluoro- methyl) nicotinalde- hyde | 561, 3.49 (A) |
| INT- 087 | 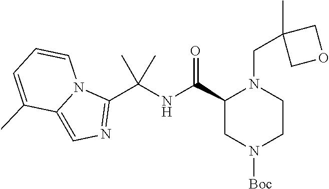 [SMILES: Cc1cccn2c(C(C)(C)NC(=O)[C@@H]3CN(C(=O)OC(C)(C)C)CCN3CC3(C)COC3)ncc12] | tert-butyl (S)-3-((2- (8-methylimidazo [1,5-a]pyridin-3- yl)propan-2-yl) carbamoyl)-4-((3- methyloxetan-3- yl)methyl)piperazine- 1-carboxylate | INT-068 and 3- methyl- oxetane-3- carbalde- hyde | 486, 3.51 (A) |
| INT- 088 | 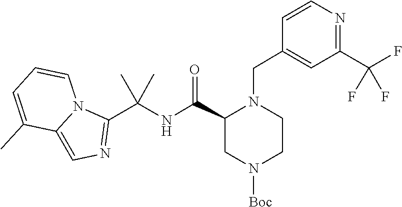 [SMILES: Cc1cccn2c(C(C)(C)NC(=O)[C@@H]3CN(C(=O)OC(C)(C)C)CCN3Cc3ccnc(C(F)(F)F)c3)ncc12] | tert-butyl (S)-3-((2-(8- methylimidazo[1,5- a]pyridin-3-yl) propan-2-yl) carbamoyl)- 4-((2-(trifluoro- methyl)pyridin- 4-yl)methyl) piperazine-1- carboxylate | INT-068 and 2- (trifluoro- methyl) isonicotin- aldehyde | 561, 1.56 (A) |
| INT- 089 | 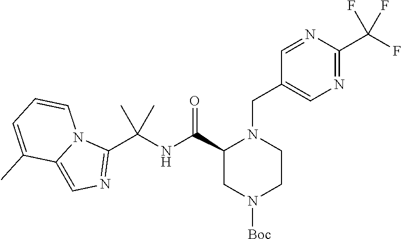 [SMILES: Cc1cccn2c(C(C)(C)NC(=O)[C@@H]3CN(C(=O)OC(C)(C)C)CCN3Cc3cnc(C(F)(F)F)nc3)ncc12] | tert-butyl (S)-3-((2-(8- methylimidazo[1,5- a]pyridin-3-yl) propan-2-yl) carbamoyl)- 4-((2-(trifluoro- methyl)pyrimidin-5- yl)methyl)piperazine- 1-carboxylate | INT-068 and 2- (trifluoro- methyl) pyrimidine- 5-carb- aldehyde | 562, 3.50 (A) |
| INT- 090 | 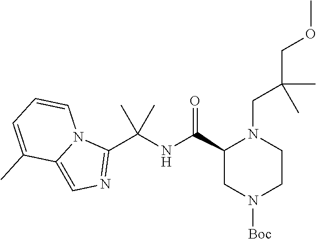 [SMILES: COCC(C)(C)CN1CCN(C(=O)OC(C)(C)C)C[C@H]1C(=O)NC(C)(C)c1ncc2c(C)cccn12] | tert-butyl (S)-4-(3- methoxy-2,2- dimethylpropyl)- 3-((2-(8- methylimidazo[1,5- a]pyridin-3-yl) propan-2-yl) carbamoyl) piperazine-1- carboxylate | INT-068 and 3- methoxy- 2,2- dimethyl- propanal | 502, 1.81 (A) |

### Synthesis of INT-091: tert-butyl (S)-4-(cyclobutylmethyl)-3-((2-(8-methylimidazo[1,5-a]pyridin-3-yl)propan-2-yl)carbamoyl)piperazine-1-carboxylate

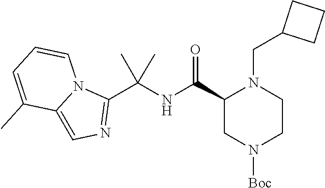 [SMILES: Cc1cccn2c(C(C)(C)NC(=O)[C@@H]3CN(C(=O)OC(C)(C)C)CCN3CC3CCC3)ncc12]

To a solution of carboxamide INT-068 (200 mg, 0.49 mmol) in THF (8 mL) were added cyclobutanecarbaldehyde (126 mg, 1.49 mmol) and AcOH (2 drops) at room temperature and the mixture was stirred for 1 h before NaBH(OAc)₃ (211 mg, 0.99 mmol) was added and stirring was continued for 16 h. The reaction was quenched by addition of aq. NaHCO₃ (50 mL) and the mixture was extracted with CH₂Cl₂ (2×50 mL). The combined organic layers were washed with H₂O (20 mL) and aq. sat. NaCl solution (20 mL), dried over anhyd. Na₂SO₄ and concentrated under reduced pressure. The residue was purified by column chromatography (silica, hexane/EtOAc 45:55-40:60) to obtain the title compound (130 mg, 56%). LCMS m/z=471 [M+H]⁺, Rₜ=1.54 min (Method A).

The following compound was prepared in analogy:

| | | | | LC-MS |
|---|---|---|---|---|
| | | | | m/z |
| | | | | [M + H]⁺, Rt |
| Inter- | | | | [min] |
| mediate | Structure | Chemical Name | Precursors | (Method) |
| INT-092 | 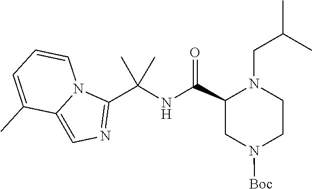 [SMILES: Cc1cccn2c(C(C)(C)NC(=O)[C@@H]3CN(C(=O)OC(C)(C)C)CCN3CC(C)C)ncc12] | tert-butyl (S)-4-isobutyl- 3-((2-(8-methylimidazo [1,5-a]pyridin-3- yl)propan-2-yl) carbamoyl) piperazine-1- carboxylate | INT-068 and iso- butyralde- hyde | 458, 3.53 (A) |

### Synthesis of INT-093: tert-butyl (S)-3-((2-(8-methylimidazo[1,5-a]pyridin-3-yl)propan-2-yl)carbamoyl)-4-((4-methyltetrahydro-2H-pyran-4-yl)methyl)piperazine-1-carboxylate

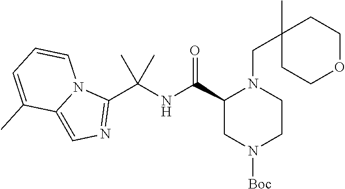 [SMILES: Cc1cccn2c(C(C)(C)NC(=O)[C@@H]3CN(C(=O)OC(C)(C)C)CCN3CC3(C)CCOCC3)ncc12]

To a solution of carboxamide INT-068 (250 mg, 0.62 mmol) in CH₂Cl₂ (15 mL) were added 4-methyltetrahydro-2H-pyran-4-carbaldehyde (120 mg, 0.93 mmol), AcOH (0.1 mL) and Na₂SO₄ (500 mg) and the mixture was stirred at room temperature for 3 h followed by addition of NaBH(OAc)₃ (394 mg, 1.87 mmol) and stirring was continued for 16 h. The mixture was diluted with CH₂Cl₂ (100 mL), washed with H₂O (50 mL) and aq. sat. NaCl solution (50 mL), dried over anhyd. Na₂SO₄ and concentrated under reduced pressure. The residue was purified by column chromatography (silica gel, CH₂Cl₂/MeOH 98:2) to obtain the title compound (70 mg, 15%). LCMS m/z=515 [M+H]⁺, Rₜ=1.88 min (Method C).

### Synthesis of INT-094: 1-benzyl 4-(tert-butyl) 2-methyl (S)-piperazine-1,2,4-tricarboxylate

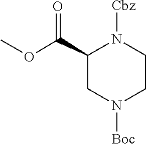 [SMILES: COC(=O)[C@@H]1CN(C(=O)OC(C)(C)C)CCN1C(=O)OCc1ccccc1]

To a solution of 1-(tert-butyl) 3-methyl (S)-piperazine-1,3-dicarboxylate (2.0 g, 8.18 mmol) in THF (10 mL) were added DIPEA (4.2 mL, 24.5 mmol), benzyl chloroformate (1.3 mL, 9.82 mmol) and the mixture was stirred at room temperature for 16 h. The mixture was extracted with EtOAc (2×10 mL) and the combined organic layers were washed with aq. sat. NaCl solution (2×10 mL) and dried over anhyd. Na₂SO₄ and concentrated under reduced pressure. The residue was purified by column chromatography (hexane/EtOH 1:0-1:1) to obtain the title compound (2.0 g, 65%). LCMS m/z=379 [M+H]⁺, Rₜ=3.55 min (Method A).

### Synthesis of INT-095: (S)-1-((benzyloxy)carbonyl)-4-(tert-butoxycarbonyl)piperazine-2-carboxylic acid

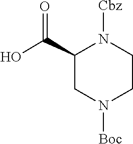 [SMILES: CC(C)(C)OC(=O)N1CCN(C(=O)OCc2ccccc2)[C@H](C(=O)O)C1]

To a stirred solution of ester INT-094 (4.0 g, 10.6 mmol) in THF/Water (10:1, 45.0 mL) was added LiOH·H₂O (1.2 g, 31.7 mmol) and the mixture was stirred at room temperature for 16 h. The mixture was acidified with aq. NaHSO₄ solution (30 mL) and extracted with EtOAc (2×50 mL). The combined organic layers were washed with aq. sat. NaCl solution (2×10 mL) and dried over anhyd. Na₂SO₄ and concentrated under reduced pressure to obtain the title compound (150 mg, 83%). LCMS m/z=365 [M+H]⁺, Rₜ=0.64 min (Method A).

### Synthesis of INT-096: 1-benzyl 4-(tert-butyl) (S)-2-((2-(7-methyl-1-((2-(trimethylsilyl)ethoxy)methyl)-1H-indazol-3-yl)propan-2-yl)carbamoyl)piperazine-1,4-dicarboxylate

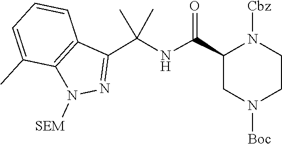 [SMILES: Cc1cccc2c(C(C)(C)NC(=O)[C@@H]3CN(C(=O)OC(C)(C)C)CCN3C(=O)OCc3ccccc3)nn(COCC[Si](C)(C)C)c12]

To a solution of carboxylic acid INT-095 (250 mg, 0.69 mmol) in DMF (5 mL) were added DIPEA (0.35 mL, 2.05 mmol), HATU (0.31 g, 0.82 mmol) and the mixture was stirred for 10 min at room temperature. Amine INT-010 (0.22 g, 0.69 mmol) was added and the mixture was continued to stir for 16 h. The mixture was extracted with EtOAc (2×10 mL) and the combined organic layers were washed with aq. sat. NaCl solution (2×10 mL), dried over anhyd. Na₂SO₄ and concentrated under reduced pressure. The residue was purified by column chromatography (silica, CH₂Cl₂/MeOH 1:0-9:1) to obtain the title compound (100 mg, 55%). LCMS m/z=666 [M+H]⁺, Rₜ=2.60 min (Method A).

### Synthesis of INT-097: tert-butyl (S)-3-((2-(7-methyl-1-((2-(trimethylsilyl)ethoxy)methyl)-1H-indazol-3-yl)propan-2-yl)carbamoyl)piperazine-1-carboxylate

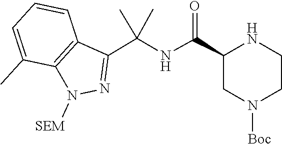 [SMILES: Cc1cccc2c(C(C)(C)NC(=O)[C@@H]3CN(C(=O)OC(C)(C)C)CCN3)nn(COCC[Si](C)(C)C)c12]

To a solution of carboxamide INT-096 (0.45 g, 0.676 mmol) in EtOAc (20 mL) was added Pd/C (200 mg) and the mixture was stirred under hydrogen atmosphere at room temperature for 16 h. The mixture was filtered through a pad of celite and concentrated under reduced pressure to obtain the title compound (0.35 g, 97%). LCMS m/z=532 [M+H]⁺, Rₜ=2.45 min (Method H).

### Synthesis of INT-098: tert-butyl (S)-4-isopropyl-3-((2-(7-methyl-1-((2-(trimethylsilyl)ethoxy)methyl)-1H-indazol-3-yl)propan-2-yl)carbamoyl)piperazine-1-carboxylate

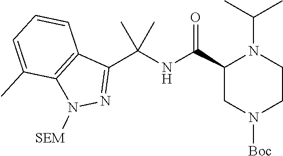 [SMILES: Cc1cccc2c(C(C)(C)NC(=O)[C@@H]3CN(C(=O)OC(C)(C)C)CCN3C(C)C)nn(COCC[Si](C)(C)C)c12]

To a solution of carboxamide INT-097 (350 mg, 0.66 mmol) in MeOH (10 mL) were added 2-propanone (10 μL, 1.31 mmol) and AcOH (0.1 mL) and the mixture was stirred for 2 h at room temperature before it was cooled to 0° C. and NaBH₃CN (125 mg, 1.97 mmol) was added. The mixture was allowed to warm to room temperature and stirred for 16 h before it was diluted with CH₂Cl₂, filtered and the filter cake washed with CH₂Cl₂. The combined organic layers were washed with H₂O (50 mL), dried over anhyd. Na₂SO₄ and concentrated under reduced pressure. The residue was purified by column chromatography (silica gel, CH₂Cl₂/MeOH 98:2-97:3) to obtain the title compound (280 mg, 74%). LCMS m/z=574 [M+H]⁺, Rₜ=2.70 min (Method A).

The following compound was prepared in analogy:

| | | | | LC-MS m/z |
|---|---|---|---|---|
| | | | | [M + H]⁺, Rt |
| Inter- | | | | [min] |
| mediate | Structure | Chemical Name | Precursors | (Method) |
| INT- 099 | 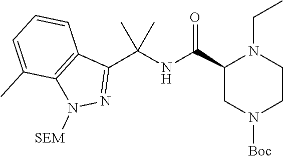 [SMILES: CCN1CCN(C(=O)OC(C)(C)C)C[C@H]1C(=O)NC(C)(C)c1nn(COCC[Si](C)(C)C)c2c(C)cccc12] | tert-butyl (S)-4-ethyl-3- ((2-(7-methyl-1-((2-(tri- methylsilyl)ethoxy)methyl)- 1H-indazol-3-yl)propan-2- yl)carbamoyl)piperazine- 1-carboxylate | INT-097 and acetaldehyde | 560, 2.72 (A) |

### Synthesis of INT-100: (S)-4-(((9H-fluoren-9-yl)methoxy)carbonyl)-1-(tert-butoxycarbonyl)piperazine-2-carboxylic acid

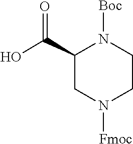

To a solution of (S)-1-(tert-butoxycarbonyl)piperazine-2-carboxylic acid (4.5 g, 19.5 mmol) in H₂O (40 mL) and 1,4-dioxane (50 mL) was added DIPEA (6.3 g, 49 mmol) at 0° C. under nitrogen atmosphere. A solution of (9H-Fluoren-9-yl)methyl carbonochloridate (6.3 g, 19.5 mmol) in 1,4-dioxane (50 mL) was added dropwise over 1 h and subsequently the resulting mixture was allowed to warm to room temperature and stirred for 12 h. The reaction was quenched by addition of aq. HCl (1N, 60 mL) and the mixture was extracted with CH₂Cl₂ (4×60 mL). The combined organic layers were concentrated under reduced pressure to obtain the title compound (10.0 g, 74%, 65% purity) which was used without further purification. ¹H NMR (400 MHz, CDCl₃) δ 7.93-7.81 (m, 2H), 7.79-7.54 (m, 2H), 7.46-7.37 (m, 2H), 7.37-7.26 (m, 2H), 4.75-4.42 (m, 1H), 4.33-4.22 (m, 4H), 3.84-3.61 (m, 4H), 3.33-2.82 (m, 4H), 1.39 (m, 9H).

### Synthesis of INT-101: 4-((9H-fluoren-9-yl)methyl) 1-(tert-butyl) (S)-2-((2-(8-methylimidazo[1,5-a]pyridin-3-yl)propan-2-yl)carbamoyl)piperazine-1,4-dicarboxylate

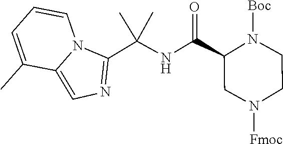

To piperazine INT-100 (16.1 g, 23.2 mmol, 65% purity) in DMF (250 mL) were added HATU (10.1 g, 26.5 mmol) and DIPEA (8.6 g, 66.4 mmol) at room temperature under nitrogen atmosphere. The mixture was stirred at that temperature for 2.5 h before amine INT-004 (5.0 g, 22.1 mmol, HCl salt) was added. Stirring at room temperature was continued for 12 h before H₂O (400 mL) was added. The mixture was extracted with EtOAc (3×500 mL) and the combined organic layers were concentrated under reduced pressure. The residue was purified by column chromatography (silica, PE/EtOAc 5:1 0:1) to obtain the title compound (11.0 g, 80%). LCMS m/z=624 [M+H]⁺; ¹H NMR (400 MHz, CDCl₃) δ 8.01 (s, 1H), 7.89 (s, 1H), 7.75 (d, 2H), 7.65-7.50 (m, 3H), 7.38 (t, 3H), 7.32-7.27 (m, 3H), 6.55-6.35 (m, 2H), 4.66-4.21 (m, 4H), 3.30-3.08 (m, 3H), 2.35 (s, 3H), 1.87-1.89 (m, 6H), 1.61-1.38 (m, 9H).

### Synthesis of INT-102: tert-butyl (S)-2-((2-(8-methylimidazo[1,5-a]pyridin-3-yl)propan-2-yl)carbamoyl)piperazine-1-carboxylate

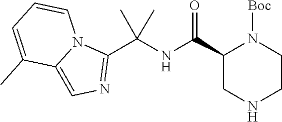 [SMILES: Cc1cccn2c(C(C)(C)NC(=O)[C@@H]3CNCCN3C(=O)OC(C)(C)C)ncc12]

To a solution of carboxamide INT-101 (9.0 g, 14.4 mmol) in CH₂Cl₂ (60 mL) was added piperidine (1.3 g, 15.2 mmol) at room temperature under nitrogen atmosphere. The mixture was stirred for 1 h at the same temperature before it was concentrated under reduced pressure. The residue was purified by column chromatography (silica, PE/EtOAc 5:1-0:1) to obtain the title compound (2.9 g, 50%). ¹H NMR (400 MHz, DMSO-d₆) δ 8.28-8.00 (m, 2H), 7.23 (s, 1H), 6.57-6.33 (m, 2H), 4.43-4.17 (m, 1H), 3.53-3.40 (m, 1H), 3.28 (s, 1H), 3.20-2.82 (m, 2H), 2.82-2.56 (m, 2H), 2.37-2.28 (m, 3H), 1.77-1.61 (m, 6H), 1.45-1.21 (m, 9H).

### Synthesis of INT-103: tert-butyl (S)-4-benzyl-3-((2-(8-methylimidazo[1,5-a]pyridin-3-yl)propan-2-yl)carbamoyl)piperazine-1-carboxylate

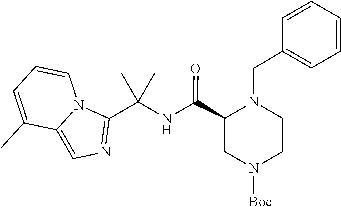 [SMILES: Cc1cccn2c(C(C)(C)NC(=O)[C@@H]3CN(C(=O)OC(C)(C)C)CCN3Cc3ccccc3)ncc12]

To a solution of carboxylic acid INT-048 (120 mg, 0.38 mmol) in DMF (3 mL) were added HATU (214 mg, 0.56 mmol), DIPEA (0.3 mL, 1.9 mmol) and amine INT-004 (85 mg, 0.38 mmol, HCl salt) at 0° C. The mixture was allowed to warm to room temperature and stirred for 16 h before it was poured into ice cold water and extracted with EtOAc (30 mL). The organic layer was washed with H₂O (25 mL), sat. aq. NaCl solution (25 mL) and dried over anhyd. Na₂SO₄. The organic layer was concentrated under reduced pressure and the residue purified by column chromatography (silica, hexane/EtOAc 1:1) to obtain the title compound (160 mg, 87%). LCMS m/z=492 [M+H]⁺, Rₜ=3.47 min (Method A).

The following compounds were prepared in analogy:

| | | | | LC-MS |
|---|---|---|---|---|
| | | | | m/z |
| | | | | [M + H]⁺, |
| Inter- | | | | Rt [min] |
| mediate | Structure | Chemical Name | Precursors | (Method) |
| INT- 104 | 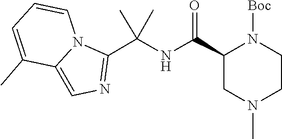 [SMILES: Cc1cccn2c(C(C)(C)NC(=O)[C@@H]3CN(C)CCN3C(=O)OC(C)(C)C)ncc12] | tert-butyl (S)-4-methyl-2- ((2-(8-methylimidazo [1,5-a]pyridin-3- yl)propan-2-yl) carbamoyl)piperazine- 1-carboxylate | INT-004 and INT-047 | 416, 2.94 (A) |
| INT- 105 | 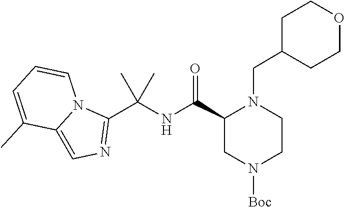 [SMILES: Cc1cccn2c(C(C)(C)NC(=O)[C@@H]3CN(C(=O)OC(C)(C)C)CCN3CC3CCOCC3)ncc12] | tert-butyl (S)-3-((2-(8- methylimidazo[1,5-a] pyridin-3-yl)propan- 2-yl)carbamoyl)- 4-((tetrahydro- 2H-pyran-4-yl)methyl) piperazine-1-carboxylate | INT-004 and INT-049 | 500, 3.16 (A) |
| INT- 106 | 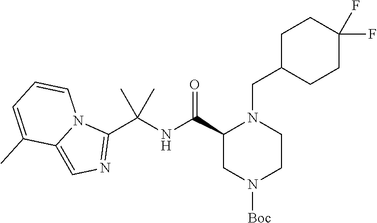 [SMILES: Cc1cccn2c(C(C)(C)NC(=O)[C@@H]3CN(C(=O)OC(C)(C)C)CCN3CC3CCC(F)(F)CC3)ncc12] | tert-butyl (S)-4-((4,4- difluorocyclohexyl) methyl)-3-((2-(8-methyl- imidazo[1,5-a]pyridin-3- yl)propan-2-yl) carbamoyl)piperazine-1- carboxylate | INT-004 and INT-050 | 534, 3.54 (A) |
| INT- 107 | 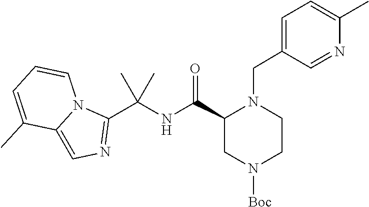 | tert-butyl (S)-3-((2-(8- methylimidazo[1,5-a] pyridin-3-yl)propan-2-yl) carbamoyl)-4-((6-methyl- pyridin-3-yl)methyl) piperazine-1-carboxylate | INT-004 and INT-051 | 507, 3.00 (A) |
| INT- 108 | 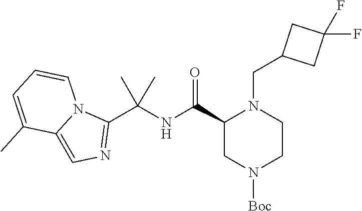 [SMILES: Cc1cccn2c(C(C)(C)NC(=O)[C@@H]3CN(C(=O)OC(C)(C)C)CCN3CC3CC(F)(F)C3)ncc12] | tert-butyl (S)-4-((3,3- difluorocyclobutyl) methyl)-3-((2-(8-methyl- imidazo[1,5-a]pyridin-3- yl)propan-2-yl) carbamoyl)piperazine- 1-carboxylate | INT-004 and INT-052 | 506, 3.44 (A) |
| INT- 109 | 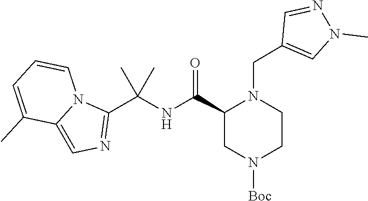 | tert-butyl (S)-4-((1- methyl-1H-pyrazol-4-yl) methyl)-3-((2-(8-methyl- imidazo[1,5-a]pyridin-3- yl)propan-2-yl) carbamoyl)piperazine- 1-carboxylate | INT-004 and INT-053 | 496, 2.94 (A) |
| INT- 110 | 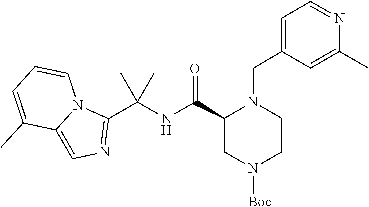 | tert-butyl (S)-3-((2-(8- methylimidazo[1,5-a] pyridin-3-yl)propan-2-yl) carbamoyl)-4-((2-methyl- pyridin-4-yl)methyl) piperazine-1-carboxylate | INT-004 and INT-054 | 507, 3.01 (A) |
| INT- 111 | 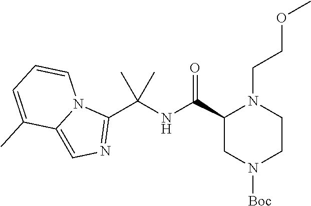 | tert-butyl (S)-4-(2- methoxyethyl)-3-((2- (8-methylimidazo [1,5-a]pyridin- 3-yl)propan-2-yl) carbamoyl)piperazine- 1-carboxylate | INT-004 and INT-055 | 460, 3.17 (A) |
| INT- 112 | 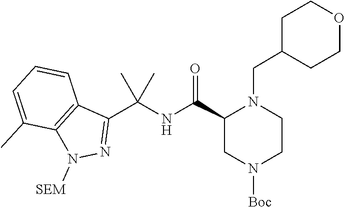 [SMILES: Cc1cccc2c(C(C)(C)NC(=O)[C@@H]3CN(C(=O)OC(C)(C)C)CCN3CC3CCOCC3)nn(COCC[Si](C)(C)C)c12] | tert-butyl (S)-3-((2-(7- methyl-1-((2-(trimethyl- silyl)ethoxy)methyl)-1H- indazol-3-yl)propan-2- yl)carbamoyl)-4-((tetra- hydro-2H-pyran-4-yl) methyl)piperazine-1- carboxylate | INT-010 and INT-049 | 630, 2.54 (A) |
| INT- 113 | 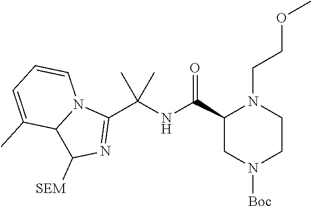 | tert-butyl (S)-4-(2- methoxyethyl)-3-((2-(7- methyl-1-((2-(trimethyl- silyl)ethoxy)methyl)-1H- indazol-3-yl)propan-2- yl)carbamoyl)piperazine- 1-carboxylate | INT-010 and INT-055 | 590, 2.55 (A) |
| INT- 114 | 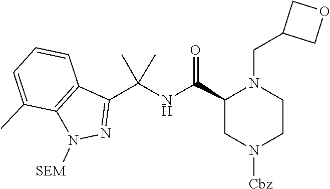 [SMILES: Cc1cccc2c(C(C)(C)NC(=O)[C@@H]3CN(C(=O)OCc4ccccc4)CCN3CC3COC3)nn(COCC[Si](C)(C)C)c12] | benzyl (S)-3-((2-(7- methyl-1-((2-(trimethyl- silyl)ethoxy)methyl)-1H- indazol-3-yl)propan-2- yl)carbamoyl)-4-(oxetan- 3-ylmethyl)piperazine-1- carboxylate | INT-010 and INT-057 | 636, 3.33 (G) |
| INT- 115 | 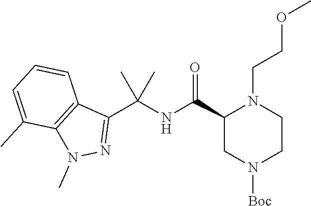 | tert-butyl (S)-4-(2- methoxyethyl)-3-((2-(1- methyl-1H-indazol-3-yl) propan-2-yl)carbamoyl) piperazine-1-carboxylate | INT-012 and INT-055 | 460, 3.41 (A) |
| INT- 116 | 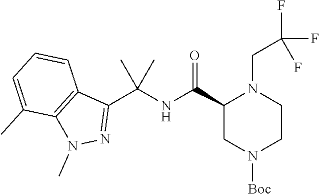 [SMILES: Cc1cccc2c(C(C)(C)NC(=O)[C@@H]3CN(C(=O)OC(C)(C)C)CCN3CC(F)(F)F)nn(C)c12] | tert-butyl (S)-3-((2-(1- methyl-1H-indazol-3- yl)propan-2-yl) carbamoyl)-4-(2,2,2- trifluoroethyl)piperazine- 1-carboxylate | INT-012 and INT-056 | 484, 3.65 (A) |
| INT- 117 | 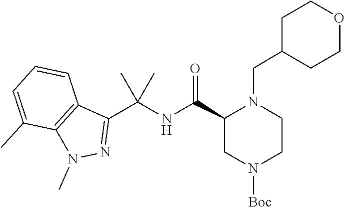 [SMILES: Cc1cccc2c(C(C)(C)NC(=O)[C@@H]3CN(C(=O)OC(C)(C)C)CCN3CC3CCOCC3)nn(C)c12] | tert-butyl (S)-3-((2-(1- methyl-1H-indazol-3- yl)propan-2-yl) carbamoyl)-4-((tetrahydro- 2H-pyran-4-yl)methyl) piperazine-1-carboxylate | INT-012 and INT-049 | 500, 3.51 (A) |
| INT- 118 | 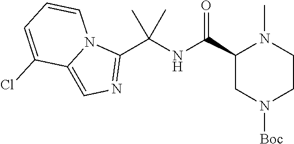 [SMILES: CN1CCN(C(=O)OC(C)(C)C)C[C@H]1C(=O)NC(C)(C)c1ncc2c(Cl)cccn12] | tert-butyl (S)-3-((2-(8- chloroimidazo[1,5-a] pyridin-3-yl)propan-2-yl) carbamoyl)-4- methylpiperazine- 1-carboxylate | INT-017 and INT-046 | 436, 3.10 (A) |
| INT- 119 | 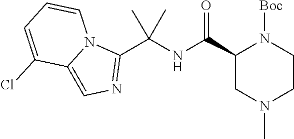 [SMILES: CN1CCN(C(=O)OC(C)(C)C)[C@H](C(=O)NC(C)(C)c2ncc3c(Cl)cccn23)C1] | tert-butyl (S)-2-((2-(8- chloroimidazo[1,5-a] pyridin-3-yl)propan-2-yl) carbamoyl)-4- methylpiperazine- 1-carboxylate | INT-017 and INT-047 | 436, 1.75 (H) |
| INT- 120 | 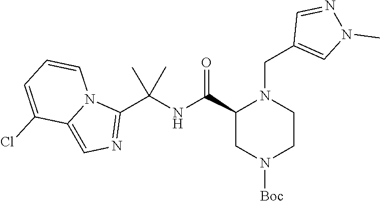 [SMILES: Cn1cc(CN2CCN(C(=O)OC(C)(C)C)C[C@H]2C(=O)NC(C)(C)c2ncc3c(Cl)cccn23)cn1] | tert-butyl (S)-3-((2-(8- chloroimidazo[1,5-a]pyri- din-3-yl)propan-2-yl)car- bamoyl)-4-((1-methyl- 1H-pyrazol-4-yl)me- thyl)piperazine-1-carbox- ylate | INT-017 and INT-053 | 516, 1.72 (H) |
| INT- 121 | 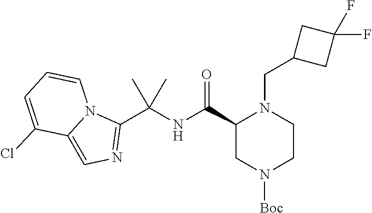 [SMILES: CC(C)(C)OC(=O)N1CCN(CC2CC(F)(F)C2)[C@H](C(=O)NC(C)(C)c2ncc3c(Cl)cccn23)C1] | tert-butyl (S)-3-((2-(8- chloroimidazo[1,5-a] pyridin-3-yl)propan- 2-yl)carbamoyl)-4- ((3,3-difluoro- cyclobutyl)methyl) piperazine-1-carboxylate | INT-017 and INT-052 | 526, 3.43 (A) |
| INT- 122 | 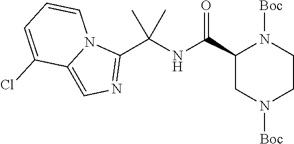 [SMILES: CC(C)(C)OC(=O)N1CCN(C(=O)OC(C)(C)C)[C@H](C(=O)NC(C)(C)c2ncc3c(Cl)cccn23)C1] | di-tert-butyl (S)-2-((2-(8- chloroimidazo[1,5-a] pyridin-3-yl)propan-2-yl) carbamoyl)piperazine- 1,4-dicarboxylate | INT-017 and (S)- 1,4- bis(tert- butoxy- carbonyl) piperazine- 2- carboxylic acid | 522, 1.55 (A) |
| INT- 123 | 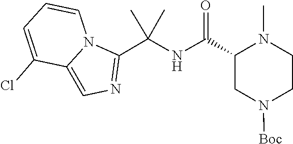 [SMILES: CN1CCN(C(=O)OC(C)(C)C)C[C@@H]1C(=O)NC(C)(C)c1ncc2c(Cl)cccn12] | tert-butyl (R)-3-((2-(8- chloroimidazo[1,5-a] pyridin-3-yl)propan-2-yl) carbamoyl)-4- methylpiperazine- 1-carboxylate | INT-017 and (R)- 4-(tert- butoxy- carbonyl)- 1- methyl- piperazine- 2- carboxylic acid | 436, 3.06 (A) |
| INT- 124 | 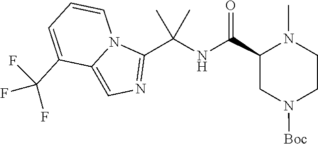 [SMILES: CN1CCN(C(=O)OC(C)(C)C)C[C@H]1C(=O)NC(C)(C)c1ncc2c(C(F)(F)F)cccn12] | tert-butyl (S)-4-methyl-3- ((2-(8-(trifluoro- methyl)imidazo[1,5-a] pyridin-3-yl)propan-2-yl) carbamoyl)piperazine- 1-carboxylate | INT-030 and INT-046 | 470, 3.20 (A) |
| INT- 125 | 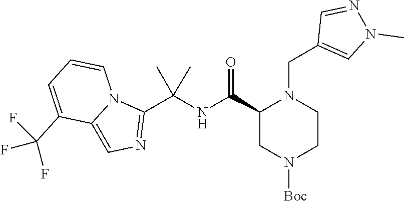 [SMILES: Cn1cc(CN2CCN(C(=O)OC(C)(C)C)C[C@H]2C(=O)NC(C)(C)c2ncc3c(C(F)(F)F)cccn23)cn1] | tert-butyl (S)-4-((1- methyl-1H-pyrazol-4-yl) methyl)-3-((2-(8-(trifluoro- methyl)imidazo[1,5- a]pyridin-3-yl)propan-2- yl)carbamoyl)piperazine- 1-carboxylate | INT-030 and INT-053 | 550, 3.13 (A) |
| INT- 126 | 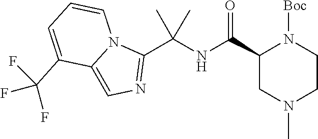 [SMILES: CN1CCN(C(=O)OC(C)(C)C)[C@H](C(=O)NC(C)(C)c2ncc3c(C(F)(F)F)cccn23)C1] | tert-butyl (S)-4-methyl-2- ((2-(8-(trifluoro- methyl)imidazo[1,5-a] pyridin-3-yl)propan-2-yl) carbamoyl)piperazine-1- carboxylate | INT-030 and INT-047 | 470, 3.73 (A) |
| INT- 127 | 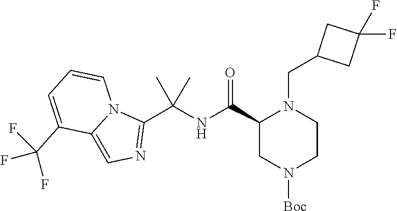 [SMILES: CC(C)(C)OC(=O)N1CCN(CC2CC(F)(F)C2)[C@H](C(=O)NC(C)(C)c2ncc3c(C(F)(F)F)cccn23)C1] | tert-butyl (S)-4-((3,3- difluorocyclobutyl) methyl)-3-((2-(8-(trifluoro- methyl)imidazo[1,5- a]pyridin-3-yl)propan-2- yl)carbamoyl)piperazine- 1-carboxylate | INT-030 and INT-052 | 560, 1.63 (A) |
| INT- 128 | 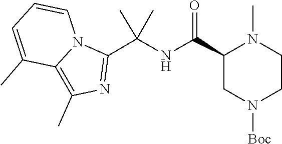 [SMILES: Cc1cccn2c(C(C)(C)NC(=O)[C@@H]3CN(C(=O)OC(C)(C)C)CCN3C)nc(C)c12] | tert-butyl (S)-3-((2-(1,8- dimethylimidazo[1,5- a]pyridin-3-yl)propan-2- yl)carbamoyl)-4- methylpiperazine-1- carboxylate | INT-031 and INT-046 | 430, 3.15 (A) |
| INT- 129 | 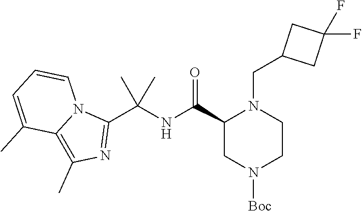 [SMILES: Cc1cccn2c(C(C)(C)NC(=O)[C@@H]3CN(C(=O)OC(C)(C)C)CCN3CC3CC(F)(F)C3)nc(C)c12] | tert-butyl (S)-4-((3,3- difluorocyclobutyl) methyl)-3-((2-(1,8- dimethylimidazo[1,5-a] pyridin-3-yl)propan-2-yl) carbamoyl)piperazine-1- carboxylate | INT-031 and INT-052 | 520, 3.46 (A) |
| INT- 130 | 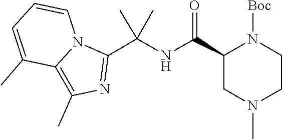 [SMILES: Cc1cccn2c(C(C)(C)NC(=O)[C@@H]3CN(C)CCN3C(=O)OC(C)(C)C)nc(C)c12] | tert-butyl (S)-2-((2-(1,8- dimethylimidazo[1,5- a]pyridin-3-yl)propan-2- yl)carbamoyl)-4- methylpiperazine-1- carboxylate | INT-031 and INT-047 | 430, 3.24 (A) |
| INT- 131 | 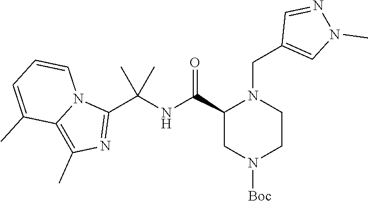 | tert-butyl (S)-3-((2-(1,8- dimethylimidazo[1,5- a]pyridin-3-yl)propan-2- yl)carbamoyl)-4-((1- methyl-1H-pyrazol-4-yl) methyl)piperazine-1- carboxylate | INT-031 and INT-053 | 510, 2.97 (A) |
| INT- 132 | 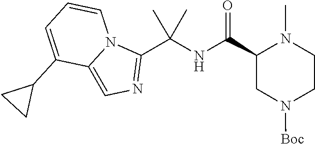 [SMILES: CN1CCN(C(=O)OC(C)(C)C)C[C@H]1C(=O)NC(C)(C)c1ncc2c(C3CC3)cccn12] | tert-butyl (S)-3-((2-(8- cyclopropylimidazo[1,5- a]pyridin-3-yl)propan-2- yl)carbamoyl)-4- methylpiperazine-1- carboxylate | INT-032 and INT-046 | 442, 3.23 (A) |
| INT- 133 | 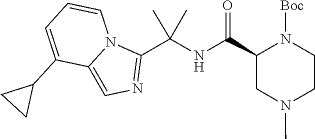 [SMILES: CN1CCN(C(=O)OC(C)(C)C)[C@H](C(=O)NC(C)(C)c2ncc3c(C4CC4)cccn23)C1] | tert-butyl (S)-2-((2-(8- cyclopropylimidazo[1,5- a]pyridin-3-yl)propan-2- yl)carbamoyl)-4- methylpiperazine-1- carboxylate | INT-032 and INT-047 | 442, 3.13 (A) |
| INT- 134 | 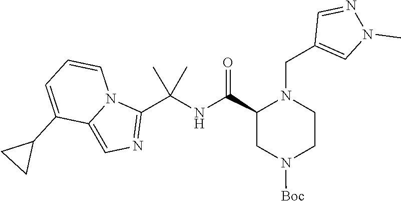 [SMILES: Cn1cc(CN2CCN(C(=O)OC(C)(C)C)C[C@H]2C(=O)NC(C)(C)c2ncc3c(C4CC4)cccn23)cn1] | tert-butyl (S)-3-((2-(8- cyclopropylimidazo[1,5- a]pyridin-3-yl)propan-2- yl)carbamoyl)-4-((1- methyl-1H-pyrazol-4-yl) methyl)piperazine-1- carboxylate | INT-032 and INT-053 | 522, 3.19 (A) |
| INT- 135 | 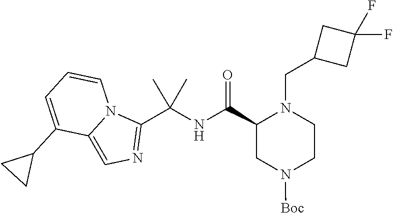 [SMILES: CC(C)(C)OC(=O)N1CCN(CC2CC(F)(F)C2)[C@H](C(=O)NC(C)(C)c2ncc3c(C4CC4)cccn23)C1] | tert-butyl (S)-3-((2-(8- cyclopropylimidazo[1,5- a]pyridin-3-yl)propan-2- yl)carbamoyl)-4-((3,3- difluorocyclobutyl) methyl)piperazine-1- carboxylate | INT-032 and INT-052 | 532, 3.50 (A) |
| INT- 136 | 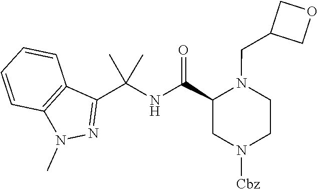 [SMILES: Cn1nc(C(C)(C)NC(=O)[C@@H]2CN(C(=O)OCc3ccccc3)CCN2CC2COC2)c2ccccc21] | benzyl (S)-3-((2-(1- methyl-1H-indazol-3-yl) propan-2-yl)carbamoyl)-4- (oxetan-3-ylmethyl) piperazine-1-carboxylate | INT-012 and INT-057 | 506, 1.23 (A) |
| INT- 144 | 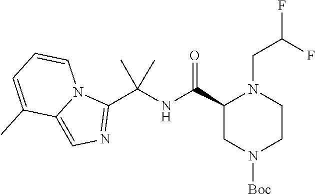 [SMILES: Cc1cccn2c(C(C)(C)NC(=O)[C@@H]3CN(C(=O)OC(C)(C)C)CCN3CC(F)F)ncc12] | tert-butyl (S)-4-(2,2- difluoroethyl)-3-((2-(8- methylimidazo[1,5-a] pyridin-3-yl)propan-2-yl) carbamoyl)piperazine-1- carboxylate | INT-004 and INT-145 | 467, 2.58 (C) |
| INT- 167 | 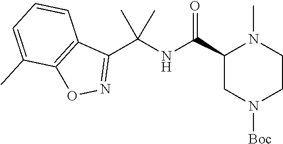 [SMILES: Cc1cccc2c(C(C)(C)NC(=O)[C@@H]3CN(C(=O)OC(C)(C)C)CCN3C)noc12] | tert-butyl (S)-4-methyl-3- ((2-(7-methylbenzo[d] isoxazol-3-yl)propan-2- yl)carbamoyl)piperazine- 1-carboxylate | INT-155 and INT-046 | 417, 0.52 (Q) |
| INT- 168 | 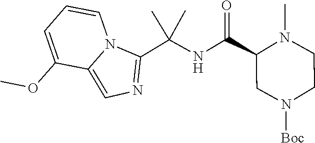 [SMILES: COc1cccn2c(C(C)(C)NC(=O)[C@@H]3CN(C(=O)OC(C)(C)C)CCN3C)ncc12] | tert-butyl (S)-3-((2-(8- methoxyimidazo[1,5- a]pyridin-3-yl)propan-2- yl)carbamoyl)-4-methyl- piperazine-1-carboxylate | INT-166 and INT-046 | 432, 0.53 (O) |
| INT- 169 | 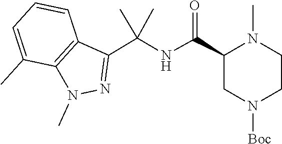 [SMILES: Cc1cccc2c(C(C)(C)NC(=O)[C@@H]3CN(C(=O)OC(C)(C)C)CCN3C)nn(C)c12] | tert-butyl (S)-3-((2-(1,7- dimethyl-1H-indazol-3- yl)propan-2- yl)carbamoyl)-4-methyl- piperazine-1-carboxylate | INT-158 and INT-046 | 430, 0.67 (O) |
| INT- 170 | 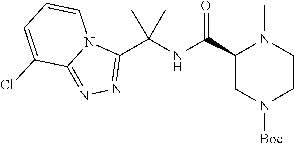 [SMILES: CN1CCN(C(=O)OC(C)(C)C)C[C@H]1C(=O)NC(C)(C)c1nnc2c(Cl)cccn12] | tert-butyl (S)-3-((2-(8- chloro-[1,2,4] triazolo[4,3-a]pyridin-3- yl)propan-2-yl) carbamoyl)-4- methylpiperazine- 1-carboxylate | INT-162 and INT-046 | 437, 1.19 (P) |

### Synthesis of TNT-137: rac-tert-butyl 3-((2-(8-methylimidazo[1,5-a]pyridin-3-yl)propan-2-yl)carbamoyl)-4-phenylpiperazine-1l-carboxylate

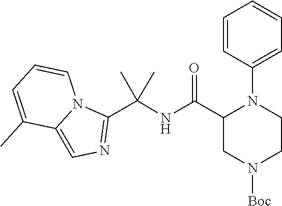 [SMILES: Cc1cccn2c(C(C)(C)NC(=O)C3CN(C(=O)OC(C)(C)C)CCN3c3ccccc3)ncc12]

To a solution of 4-(tert-butoxycarbonyl)-1-phenylpiperazine-2-carboxylic acid (75 mg, 0.25 mmol) in DMF (1 mL) were added HATU (140 mg, 0.37 mmol), Et₃N (0.1 mL, 0.74 mmol) at 0° C. and the mixture was stirred for 15 min before amine INT-004 (63 mg, 0.25 mmol, HCl salt) was added. The mixture was allowed to warm to room temperature and stirred for 16 h and diluted with EtOAc (20 mL) and H₂O (10 mL). The organic layer was separated and washed with aq. sat. NaCl solution (10 mL), dried over anhyd. Na₂SO₄ and concentrated under reduced pressure to obtain the title compound (105 mg, 89%) which was used without further purification. LCMS m/z=478 [M+H]⁺, Rₜ=1.81 min (Method C).

### Synthesis of INT-138: tert-butyl (S)-4-methyl-3-((2-(7-methyl-1-((2-(trimethylsilyl)ethoxy)methyl)1H-indazol-3-yl)propan-2-yl)carbamoyl)piperazine-1-carboxylate

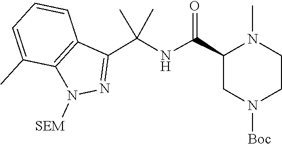 [SMILES: Cc1cccc2c(C(C)(C)NC(=O)[C@@H]3CN(C(=O)OC(C)(C)C)CCN3C)nn(COCC[Si](C)(C)C)c12]

To a suspension of amine INT-010 (200 mg, 0.62 mmol) in CH₂Cl₂ (10 mL) were added DIPEA (0.5 mL, 3.1 mmol), HATU (353 mg, 0.93 mmol) and carboxylic acid INT-046 (153 mg, 0.62 mmol) at 0° C. The mixture was allowed to warm to room temperature und was stirred for 16 h before it was diluted with CH₂Cl₂ (150 mL). The mixture was washed with H₂O (60 mL) and aq. sat. NaCl solution (60 mL) and the organic layer was dried over anhyd. Na₂SO₄ and concentrated under reduced pressure. The residue was purified by column chromatography (silica, hexane/EtOAc 60:40-55:45) to obtain the title compound (210 mg, 61%). LCMS m/z=546 [M+H]⁺, Rₜ=2.33 min (Method A).

The following compounds were prepared in analogy:

| | | | | LC-MS |
|---|---|---|---|---|
| | | | | m/z |
| | | | | [M + H]⁺, |
| Inter- | | | | Rt [min] |
| mediate | Structure | Chemical Name | Precursors | (Method) |
| INT- 139 | 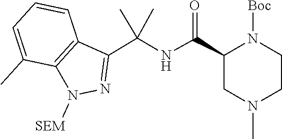 [SMILES: Cc1cccc2c(C(C)(C)NC(=O)[C@@H]3CN(C)CCN3C(=O)OC(C)(C)C)nn(COCC[Si](C)(C)C)c12] | tert-butyl (S)-4-methyl-2- ((2-(7-methyl-1-((2-(tri- methylsilyl)ethoxy)methyl)- 1H-indazol-3-yl)propan-2- yl)carbamoyl)piperazine-1- carboxylate | INT- 010 and INT- 047 | 547, 2.54 (H) |
| INT- 140 | 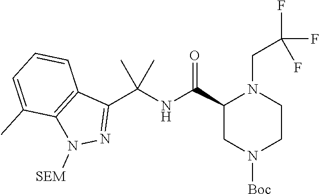 [SMILES: Cc1cccc2c(C(C)(C)NC(=O)[C@@H]3CN(C(=O)OC(C)(C)C)CCN3CC(F)(F)F)nn(COCC[Si](C)(C)C)c12] | tert-butyl (S)-3-((2-(7- methyl-1-((2-(trimethylsilyl) ethoxy)methyl)-1H-indazol- 3-yl)propan-2-yl) carbamoyl)-4- (2,2,2-trifluoro- ethyl)piperazine-1- carboxylate | INT- 010 and INT- 056 | 614, 4.17 (A) |
| INT- 141 | 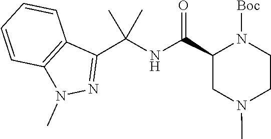 [SMILES: CN1CCN(C(=O)OC(C)(C)C)[C@H](C(=O)NC(C)(C)c2nn(C)c3ccccc23)C1] | tert-butyl (S)-4-methyl-2- ((2-(1-methyl-1H-indazol-3- yl)propan-2-yl) carbamoyl)piperazine- 1-carboxylate | INT- 012 and INT- 047 | 416, 1.58 (C) |
| INT- 142 | 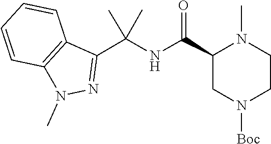 [SMILES: CN1CCN(C(=O)OC(C)(C)C)C[C@H]1C(=O)NC(C)(C)c1nn(C)c2ccccc12] | tert-butyl (S)-4-methyl-3- ((2-(1-methyl-1H-indazol-3- yl)propan-2-yl) carbamoyl)piperazine- 1-carboxylate | INT- 012 and INT- 046 | 416, 1.58 (C) |

### Synthesis of INT-143: (S)—N-(2-(7-methyl-1-((2-(trimethylsilyl)ethoxy)methyl)-1H-indazol-3-yl)propan-2-yl)-1-(oxetan-3-ylmethyl)piperazine-2-carboxamide

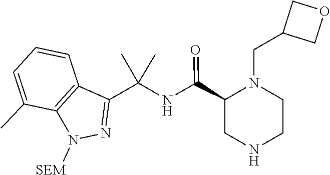 [SMILES: Cc1cccc2c(C(C)(C)NC(=O)[C@@H]3CNCCN3CC3COC3)nn(COCC[Si](C)(C)C)c12]

To a solution of INT-114 (160 mg, 0.25 mmol) in MeOH (5 mL) was added Pd/C (80 mg) and the mixture was stirred under hydrogen atmosphere at room temperature for 2 h. The mixture was filtered through a pad of celite und concentrated under reduced pressure to yield the title compound (110 mg, 87%) which was used without further purification. LCMS m/z=503 [M+H]⁺, Rₜ=1.44 min (Method A).

### Synthesis of INT-171: tert-butyl (S)-3-((2-(7-fluoro-1H-indazol-3-yl)propan-2-yl)carbamoyl)-4-methylpiperazine-1-carboxylate

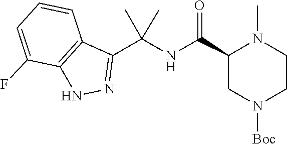 [SMILES: CN1CCN(C(=O)OC(C)(C)C)C[C@H]1C(=O)NC(C)(C)c1n[nH]c2c(F)cccc12]

To a stirred solution of INT-046 (179 mg, 0.77 mmol) and DIPEA (200 mg, 1.6 mmol) in CH₂Cl₂ (2.5 mL) under nitrogen atmosphere was added propanephosphonic acid anhydride (50 wt. % in EtOAc, 0.46 mL, 0.77 mmol) and the mixture was stirred at room temperature for 30 min. Amine INT-149 (100 mg, 0.52 mmol) in CH₂Cl₂ (2.5 mL) was added and the mixture stirred for 16 hours at room temperature. The mixture was diluted with EtOAc (15 mL) and washed with aq. sat. NaHCO₃ solution (6 mL). The layers were separated and the aqueous layer was washed with EtOac (10 mL). The combined organic layers were dried over MgSO₄ and concentrated under reduced pressure to obtain the title compound (200 mg, 95%) which was used in the next step without further purification. LCMS m/z=420 [M+H]⁺, Rₜ=0.63 min (Method O).

### Synthesis of Products:

### Synthesis of SC-001: rac-N-(2-(8-methylimidazo[1,5-a]pyridin-3-yl)propan-2-yl)-1-(pyrimidin-2-ylmethyl)piperazine-2-carboxamide

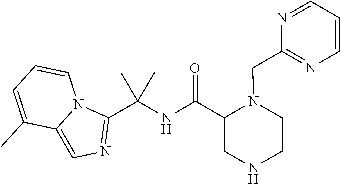 [SMILES: Cc1cccn2c(C(C)(C)NC(=O)C3CNCCN3Cc3ncccn3)ncc12]

To a solution of carboxamide INT-058 (180 mg, 0.45 mmol) in MeOH (3 mL) in a vial were added Et₃N (0.2 mL) and AcOH (0.1 mL) at room temperature. Pyrimidine-2-carbaldehyde (52 mg, 0.48 mmol) and NaBH₃CN (50 mg, 0.81 mmol) were added and the vial was sealed and the mixture shaken at room temperature for 12 h. The mixture was concentrated under reduced pressure and the residue was subjected to the next step without further purification.

To a solution of above crude carboxamide in CH₂Cl₂ (3 mL) was added TFA (0.5 mL) at room temperature and the flask was sealed and shaken for 4 h at that temperature. The mixture was concentrated under reduced pressure and the residue purified by preparative HPLC (Phenomenex Luna C18 (150×40 mm, 5μ), elution with 10 mM NH₄HCO₃ in H₂O/MeCN (92:8-62:38) at ambient temperature) to obtain the title compound (50 mg, 32%). LCMS m/z=394 [M+H]⁺; ¹H NMR (400 MHz, methanol-d₄) δ 8.87 (d, 2H), 8.22 (d, 1H), 7.50 (t, 1H), 7.39-7.31 (m, 1H), 6.55 (d, 1H), 6.45 (t, 1H), 4.11 (d, 1H), 3.95 (d, 1H), 3.52-3.47 (m, 2H), 3.24-3.15 (m, 2H), 3.09-3.00 (m, 2H), 2.75-2.64 (m, 1H), 2.42 (s, 3H), 1.90 (d, 6H).

The following compounds were prepared in analogy:

| | | | | LC-MS m/z |
|---|---|---|---|---|
| | | | | [M + H]⁺, Rt |
| | | | | [min] |
| Example | Structure | Chemical Name | Precursor | (Method) |
| SC- 002 | 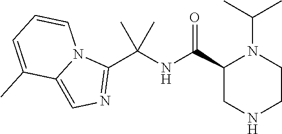 [SMILES: Cc1cccn2c(C(C)(C)NC(=O)[C@@H]3CNCCN3C(C)C)ncc12] | rac-1-isopropyl-N-(2- (8-methylimidazo[1,5- a]pyridin-3-yl)propan- 2-yl)piperazine-2- carboxamide | INT-058 and propan-2- one | 344, 1.25 (M) |
| SC- 003 | 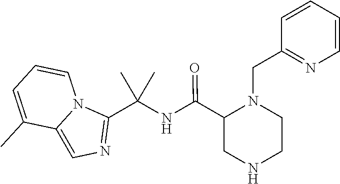 [SMILES: Cc1cccn2c(C(C)(C)NC(=O)C3CNCCN3Cc3ccccn3)ncc12] | rac-N-(2-(8-methyl- imidazo[1,5-a]pyridin-3- yl)propan-2-yl)-1- (pyridin-2-ylmethyl) piperazine-2- carboxamide | INT-058 and picolin- aldehyde | 393, 2.40 (M) |
| SC- 004 | 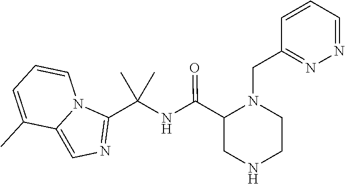 [SMILES: Cc1cccn2c(C(C)(C)NC(=O)C3CNCCN3Cc3cccnn3)ncc12] | rac-N-(2-(8-methyl- imidazo[1,5-a]pyridin-3- yl)propan-2-yl)-1- (pyridazin-3-ylmethyl) piperazine-2- carboxamide | INT-058 and pyridazine- 3-carbalde- hyde | 394, 2.10 (M) |
| SC- 005 | 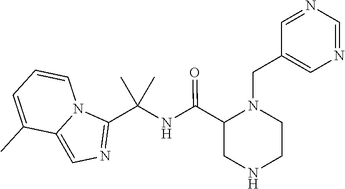 [SMILES: Cc1cccn2c(C(C)(C)NC(=O)C3CNCCN3Cc3cncnc3)ncc12] | rac-N-(2-(8-methyl- imidazo[1,5-a]pyridin-3- yl)propan-2-yl)-1- (pyrimidin-5-ylmethyl) piperazine-2- carboxamide | INT-058 and pyrimidine- 5- carbalde- hyde | 394, 2.12 (M) |
| SC- 006 | 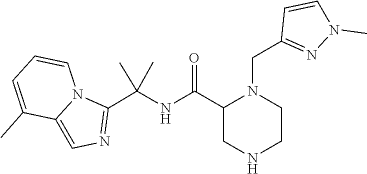 | rac-1-((1-methyl-1H- pyrazol-3-yl)methyl)- N-(2-(8-methylimidazo [1,5-a]pyridin-3- yl)propan-2-yl) piperazine-2- carboxamide | INT-058 and 1-methyl- 1H- pyrazole-3- carbalde- hyde | 396, 2.27 (M) |
| SC- 007 | 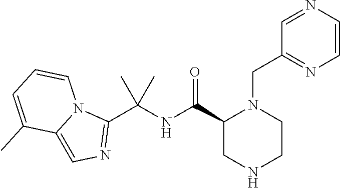 [SMILES: Cc1cccn2c(C(C)(C)NC(=O)[C@@H]3CNCCN3Cc3cnccn3)ncc12] | rac-N-(2-(8-methyl- imidazo[1,5-a]pyridin-3- yl)propan-2-yl)-1- (pyrazin-2-ylmethyl) piperazine-2- carboxamide | INT-058 and pyrazine- 2-carbalde- hyde | 394, 2.20 (M) |
| SC- 008 | 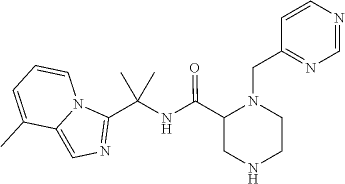 [SMILES: Cc1cccn2c(C(C)(C)NC(=O)C3CNCCN3Cc3ccncn3)ncc12] | rac-N-(2-(8-methyl- imidazo[1,5-a]pyridin-3- yl)propan-2-yl)-1- (pyrimidin-4-ylmethyl) piperazine-2- carboxamide | INT-058 and pyrimidine- 4- carbalde- hyde | 394, 1.99 (M) |

### Synthesis of SC-009: rac-1-acetyl-N-(2-(8-methylimidazo[1,5-a]pyridin-3-yl)propan-2-yl)piperazine-2-carboxamide

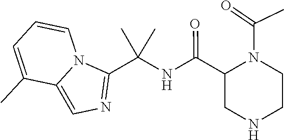 [SMILES: CC(=O)N1CCNCC1C(=O)NC(C)(C)c1ncc2c(C)cccn12]

To a solution of carboxamide INT-058 (180 mg, 0.45 mmol) in CH₂Cl₂ (3 mL) in a glass vial were added DIPEA (0.2 mL) and acetic anhydride (100 mg, 0.98 mmol) at room temperature. The vial was sealed and the mixture shaken at room temperature for 4 h. The mixture was concentrated under reduced pressure and the residue was subjected to the next step without further purification.

To a solution of above crude carboxamide in CH₂Cl₂ (3 mL) was added TFA (0.5 mL) at room temperature and the flask was sealed and shaken for 4 h at that temperature. The mixture was concentrated under reduced pressure and the residue purified by preparative HPLC (Phenomenex Luna C18 (150×40 mm, 5μ), elution with 10 mM NH₄HCO₃ in H₂O/MeCN (92:8-62:38) at ambient temperature) to obtain the title compound (115 mg, 76%). LCMS m/z=344 [M+H]⁺; ¹H NMR (400 MHz, methanol-d₄) δ 9.01 (s, 1H), 8.31 (d, 1H), 8.13 (s, 1H), 7.19-7.01 (m, 2H), 5.33 (d, 1H), 4.01 (d, 1H), 3.74-3.60 (m, 1H), 3.49-3.40 (m, 1H), 3.40-3.34 (m, 1H), 3.32-3.23 (m, 2H), 3.22-3.13 (m, 1H), 2.55 (s, 3H), 2.21 (s, 3H), 2.05-1.89 (m, 6H).

### Synthesis of SC-010: rac-1-(1-methyl-1H-pyrazole-4-carbonyl)-N-(2-(8-methylimidazo[1,5-a]pyridin-3-yl)propan-2-yl)piperazine-2-carboxamide

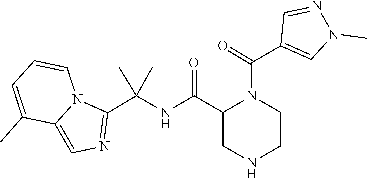

To a solution of carboxamide INT-058 (180 mg, 0.45 mmol) in DMF (3 mL) in a glass vial were added DIPEA (0.2 mL), 1-methyl-1H-pyrazole-4-carboxylic acid (100 mg, 0.79 mmol) and HATU (170 mg, 0.45 mmol) at room temperature. The vial was sealed and the mixture shaken at room temperature for 12 h. The mixture was concentrated under reduced pressure and the residue was subjected to the next step without further purification.

To a solution of above crude carboxamide in CH₂Cl₂ (3 mL) was added TFA (0.5 mL) at room temperature and the flask was sealed and shaken for 4 h at that temperature. The mixture was concentrated under reduced pressure and the residue purified by preparative HPLC (Phenomenex Luna C18 (150×40 mm, 5μ), elution with 10 mM NH₄HCO₃ in H₂O/MeCN (92:8-62:38) at ambient temperature) to obtain the title compound (151 mg, 82%). LCMS m/z=410 [M+H]⁺; ¹H NMR (400 MHz, DMSO-d₆) δ 10.21-10.10 (m, 1H), 9.49 (s, 1H), 8.92 (s, 1H), 8.43 (d, 1H), 8.24 (s, 1H), 8.11 (s, 1H), 7.66 (s, 1H), 7.07-6.90 (m, 2H), 5.15 (s, 1H), 4.13 (d, 1H), 3.87 (s, 3H), 3.69-3.57 (m, 1H), 3.30 (d, 1H), 3.17-3.12 (m, 1H), 3.00 (d, 1H), 2.46 (s, 3H), 1.92 (s, 3H), 1.82 (s, 3H).

The following compounds were prepared in analogy:

| | | | | LC-MS m/z |
|---|---|---|---|---|
| | | | | [M + H]⁺, Rt |
| | | | | [min] |
| Example | Structure | Chemical Name | Precursor | (Method) |
| SC- 011 | 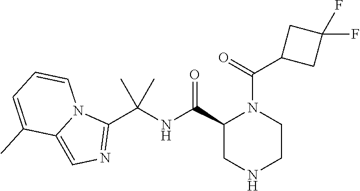 [SMILES: Cc1cccn2c(C(C)(C)NC(=O)[C@@H]3CNCCN3C(=O)C3CC(F)(F)C3)ncc12] | rac-1-(3,3-difluoro- cyclobutane-1-carbonyl)- N-(2-(8-methylimidazo [1,5-a]pyridin-3- yl)propan-2-yl) piperazine-2- carboxamide] | INT-058 and 3,3-difluoro- cyclo- butane-1- carboxylic acid | 420, 1.52 (M) |
| SC- 012 | 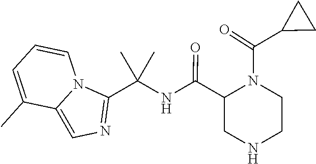 [SMILES: Cc1cccn2c(C(C)(C)NC(=O)C3CNCCN3C(=O)C3CC3)ncc12] | rac-1-(cyclopropane- carbonyl)-N- (2-(8-methylimidazo [1,5-a]pyridin- 3-yl)propan-2-yl) piperazine-2- carboxamide | INT-058 and cyclo- propane- carboxylic acid | 370, 1.31 (M) |

### Synthesis of SC-013: rac-N-(2-(8-methylimidazo[1,5-a]pyridin-3-yl)propan-2-yl)-1-(oxetane-3-carbonyl)piperazine-2-carboxamide

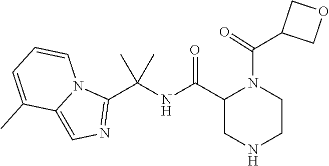 [SMILES: Cc1cccn2c(C(C)(C)NC(=O)C3CNCCN3C(=O)C3COC3)ncc12]

To a solution of carboxamide INT-058 (180 mg, 0.45 mmol) in DMF (3 mL) in a glass vial were added DIPEA (0.2 mL), oxetane-3-carboxylic acid (100 mg, 0.98 mmol) and HATU (170 mg, 0.45 mmol) at room temperature. The vial was sealed and the mixture shaken at room temperature for 12 h. The mixture was concentrated under reduced pressure and the residue was subjected to the next step without further purification.

A solution of above crude carboxamide in H₂O (3 mL) in a glass vial was sealed and heated to 100° C. for 12 h with shaking. The mixture was concentrated under reduced pressure and purified by preparative HPLC (Phenomenex Luna C18 (150×40 mm, 5μ), elution with 10 mM NH₄HCO₃ in H₂O/MeCN (92:8-62:38) at ambient temperature) to obtain the title compound (25 mg, 14%). LCMS m/z=386 [M+H]⁺; ¹H NMR (400 MHz, methanol-d₄) δ 8.14 (d, 1H), 7.33 (s, 1H), 6.68-6.51 (m, 2H), 5.00 (d, 1H), 4.85 (d, 2H), 4.82-4.69 (m, 2H), 4.24-4.09 (m, 1H), 3.44-3.37 (m, 1H), 3.29-3.22 (m, 1H), 3.03-2.92 (m, 2H), 2.75-2.68 (m, 1H), 2.41 (s, 3H), 1.92-1.73 (m, 6H).

### Synthesis of SC-014: rac-N-(2-(8-methylimidazo[1,5-a]pyridin-3-yl)propan-2-yl)-1-(oxetan-3-ylmethyl)piperazine-2-carboxamide

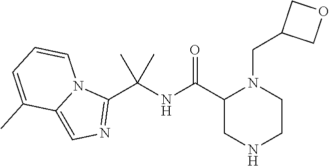 [SMILES: Cc1cccn2c(C(C)(C)NC(=O)C3CNCCN3CC3COC3)ncc12]

To a solution of carboxamide INT-058 (180 mg, 0.45 mmol) in MeOH (3 mL) in a vial were added Et₃N (0.2 mL) and AcOH (0.1 mL) at room temperature. Oxetane-3-carbaldehyde (41 mg, 0.48 mmol) and NaBH₃CN (50 mg, 0.81 mmol) were added and the vial was sealed and the mixture shaken at room temperature for 12 h. The mixture was concentrated under reduced pressure and the residue was subjected to the next step without further purification.

A solution of above crude carboxamide in H₂O (3 mL) in a glass vial was sealed and heated to 100° C. for 12 h with shaking. The mixture was concentrated under reduced pressure and purified by preparative HPLC (Phenomenex Luna C18 (150×40 mm, 5μ), elution with 10 mM NH₄HCO₃ in H₂O/MeCN (92:8-62:38) at ambient temperature) to obtain the title compound (25 mg, 15%). LCMS m/z=372 [M+H]⁺; ¹H NMR (400 MHz, methanol-d₄) δ 8.15 (d, 1H), 7.34 (s, 1H), 6.68-6.48 (m, 2H), 4.81-4.63 (m, 2H), 4.38-4.21 (m, 2H), 3.28-3.17 (m, 1H), 2.94 (dd, 1H), 2.89-2.67 (m, 6H), 2.49-2.35 (m, 4H), 2.14-2.00 (m, 1H), 1.92-1.76 (m, 6H).

### Synthesis of SC-015: (S)—N-(2-(8-methylimidazo[1,5-a]pyridin-3-yl)propan-2-yl)piperazine-2-carboxamide

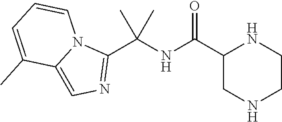 [SMILES: Cc1cccn2c(C(C)(C)NC(=O)C3CNCCN3)ncc12]

To a solution carboxamide INT-068 (160 mg, 0.37 mmol) in CH₂Cl₂ (13.5 mL) was added TFA (1.5 mL) at 0° C. and stirred at room temperature for 16 h. The mixture was concentrated under reduced pressure and the residue was purified by preparative HPLC (YMC-Actus Triart C18 (250×20 mm, 5μ), elution with 20 mM NH₄HCO₃ in H₂O/MeCN (90:10-5:95) at ambient temperature) to obtain the title compound (40 mg, 35%). LCMS m/z=302 [M+H]⁺; ¹H NMR (400 MHz, DMSO-d₆) δ 8.13 (t, 1H), 8.01 (s, 1H), 7.23 (s, 1H), 6.52-6.49 (m, 2H), 3.07 (dd, 1H), 2.81 (dd, 1H), 2.76-2.73 (m, 1H), 2.65-2.58 (m, 1H), 2.57-2.52 (m, 1H), 2.47-2.41 (m, 1H), 2.36-2.31 (m, 5H), 1.68 (s, 3H), 1.66 (s, 3H).

The following compounds were prepared in analogy:

| | | | | LC-MS m/z |
|---|---|---|---|---|
| Ex- | | | Pre- | [M + H]⁺, Rt |
| am- | | | cur- | [min] (Me- |
| ple | Structure | Chemical Name | sor | thod) |
| SC- 016 | 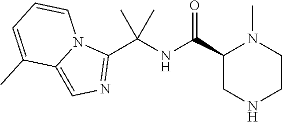 [SMILES: Cc1cccn2c(C(C)(C)NC(=O)[C@@H]3CNCCN3C)ncc12] | (S)-1-methyl-N-(2-(8- methylimidazo[1,5- a]pyridin-3-yl)propan-2- yl)piperazine-2-carbox- amide | INT- 069 | 316, 1.78 (I) |
| SC- 017 | 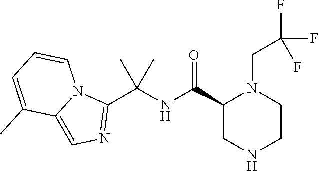 [SMILES: Cc1cccn2c(C(C)(C)NC(=O)[C@@H]3CNCCN3CC(F)(F)F)ncc12] | (S)-N-(2-(8-methylimid- azo[1,5-a]pyridin-3- yl)propan-2-yl)-1-(2,2,2- trifluoroethyl)piperazine- 2-carboxamide | INT- 070 | 384, 2.17 (I) |
| SC- 018 | 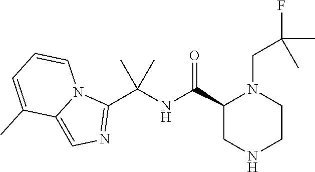 [SMILES: Cc1cccn2c(C(C)(C)NC(=O)[C@@H]3CNCCN3CC(C)(C)F)ncc12] | (S)-1-(2-fluoro-2- methylpropyl)-N-(2-(8- methylimidazo[1,5- a]pyridin-3-yl)propan-2- yl)piperazine-2-carbox- amide | INT- 071 | 376, 2.14 (I) |
| SC- 019 | 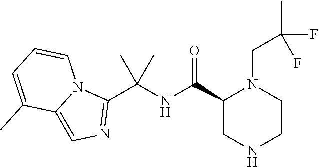 [SMILES: Cc1cccn2c(C(C)(C)NC(=O)[C@@H]3CNCCN3CC(C)(F)F)ncc12] | (S)-1-(2,2-difluoropro- pyl)-N-(2-(8-methylim- idazo[1,5-a]pyridin-3- yl)propan-2-yl)pipera- zine-2-carboxamide | INT- 072 | 380, 2.13 (I) |
| SC- 020 | 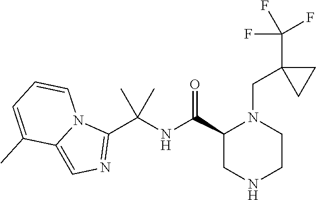 [SMILES: Cc1cccn2c(C(C)(C)NC(=O)[C@@H]3CNCCN3CC3(C(F)(F)F)CC3)ncc12] | (S)-N-(2-(8-methylimid- azo[1,5-a]pyridin-3- yl)propan-2-yl)-1-((1- (trifluoromethyl)cyclo- propyl)methyl)pipera- zine-2-carboxamide | INT- 073 | 424, 2.29 (I) |
| SC- 021 | 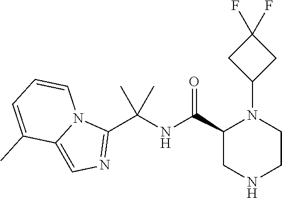 [SMILES: Cc1cccn2c(C(C)(C)NC(=O)[C@@H]3CNCCN3C3CC(F)(F)C3)ncc12] | (S)-1-(3,3-difluorocyclo- butyl)-N-(2-(8-me- thylimidazo[1,5-a]pyri- din-3-yl)propan-2-yl)pi- perazine-2-carboxamide | INT- 074 | 392, 2.09 (I) |
| SC- 022 | 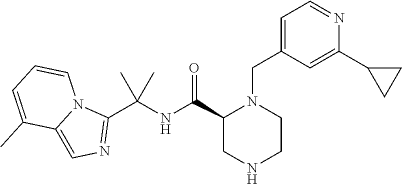 [SMILES: Cc1cccn2c(C(C)(C)NC(=O)[C@@H]3CNCCN3Cc3ccnc(C4CC4)c3)ncc12] | (S)-1-((2-cyclo- propylpyridin-4-yl)me- thyl)-N-(2-(8-methylim- idazo[1,5-a]pyridin-3- yl)propan-2-yl)pipera- zine-2-carboxamide | INT- 075 | 433, 2.12 (I) |
| SC- 023 | 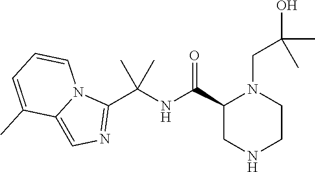 [SMILES: Cc1cccn2c(C(C)(C)NC(=O)[C@@H]3CNCCN3CC(C)(C)O)ncc12] | (S)-1-(2-hydroxy-2- methylpropyl)-N-(2-(8- methylimidazo[1,5- a]pyridin-3-yl)propan-2- yl)piperazine-2-carbox- amide | INT- 076 | 374, 1.93 (I) |
| SC- 024 | 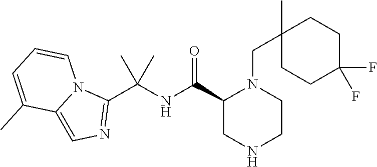 [SMILES: Cc1cccn2c(C(C)(C)NC(=O)[C@@H]3CNCCN3CC3(C)CCC(F)(F)CC3)ncc12] | (S)-1-((4,4-difluoro-1- methylcyclohexyl)me- thyl)-N-(2-(8-methylim- idazo[1,5-a]pyridin-3- yl)propan-2-yl)pipera- zine-2-carboxamide | INT- 077 | 448, 2.15 (J) |
| SC- 025 | 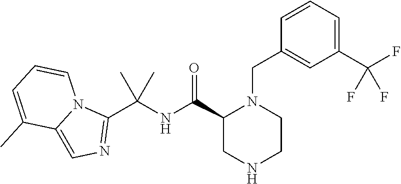 [SMILES: Cc1cccn2c(C(C)(C)NC(=O)[C@@H]3CNCCN3Cc3cccc(C(F)(F)F)c3)ncc12] | (S)-N-(2-(8-methylimid- azo[1,5-a]pyridin-3- yl)propan-2-yl)-1-(3-(tri- fluoromethyl)benzyl)pi- perazine-2-carboxamide | INT- 078 | 460, 2.56 (F) |
| SC- 026 | 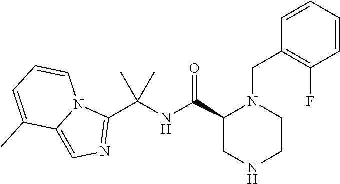 [SMILES: Cc1cccn2c(C(C)(C)NC(=O)[C@@H]3CNCCN3Cc3ccccc3F)ncc12] | (S)-1-(2-fluorobenzyl)- N-(2-(8-methylimid- azo[1,5-a]pyridin-3- yl)propan-2-yl)pipera- zine-2-carboxamide | INT- 082 | 410, 2.29 (I) |
| SC- 027 | 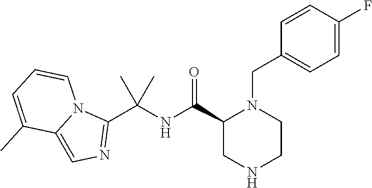 [SMILES: Cc1cccn2c(C(C)(C)NC(=O)[C@@H]3CNCCN3Cc3ccc(F)cc3)ncc12] | (S)-1-(4-fluorobenzyl)- N-(2-(8-methylimid- azo[1,5-a]pyridin-3- yl)propan-2-yl)pipera- zine-2-carboxamide | INT- 083 | 410, 2.32 (F) |
| SC- 028 | 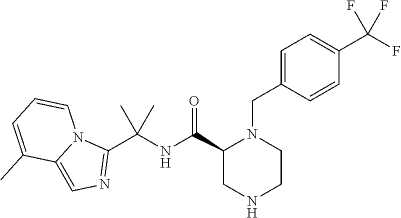 [SMILES: Cc1cccn2c(C(C)(C)NC(=O)[C@@H]3CNCCN3Cc3ccc(C(F)(F)F)cc3)ncc12] | (S)-N-(2-(8-methylimid- azo[1,5-a]pyridin-3- yl)propan-2-yl)-1-(4-(tri- fluoromethyl)benzyl)pi- perazine-2-carboxamide | INT- 084 | 460, 2.66 (I) |
| SC- 029 | 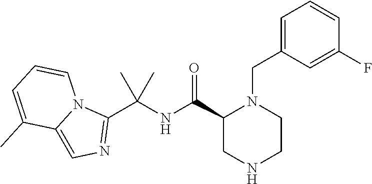 [SMILES: Cc1cccn2c(C(C)(C)NC(=O)[C@@H]3CNCCN3Cc3cccc(F)c3)ncc12] | (S)-1-(3-fluorobenzyl)- N-(2-(8-methylimid- azo[1,5-a]pyridin-3- yl)propan-2-yl)pipera- zine-2-carboxamide | INT- 085 | 410, 2.29 (I) |
| SC- 030 | 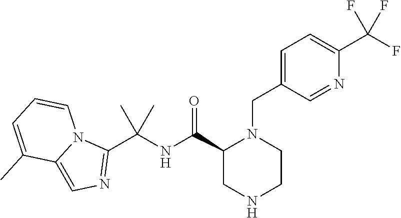 [SMILES: Cc1cccn2c(C(C)(C)NC(=O)[C@@H]3CNCCN3Cc3ccc(C(F)(F)F)nc3)ncc12] | (S)-N-(2-(8-methylimid- azo[1,5-a]pyridin-3- yl)propan-2-yl)-1-((6- (trifluoromethyl)pyridin- 3-yl)methyl)piperazine- 2-carboxamide | INT- 086 | 461, 2.26 (I) |
| SC- 031 | 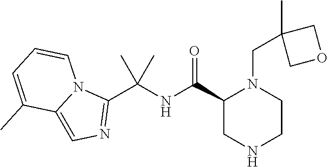 [SMILES: Cc1cccn2c(C(C)(C)NC(=O)[C@@H]3CNCCN3CC3(C)COC3)ncc12] | (S)-N-(2-(8-methylimid- azo[1,5-a]pyridin-3- yl)propan-2-yl)-1-((3- methyloxetan-3-yl)me- thyl)piperazine-2-car- boxamide | INT- 087 | 386, 2.10 (I) |
| SC- 032 | 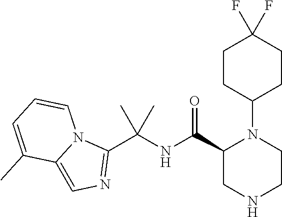 [SMILES: Cc1cccn2c(C(C)(C)NC(=O)[C@@H]3CNCCN3C3CCC(F)(F)CC3)ncc12] | (S)-1-(4,4-difluorocyclo- hexyl)-N-(2-(8-me- thylimidazo[1,5-a]pyri- din-3-yl)propan-2-yl)pi- perazine-2-carboxamide | INT- 079 | 418 [M − H]⁻, 2.14 (I) |
| SC- 033 | 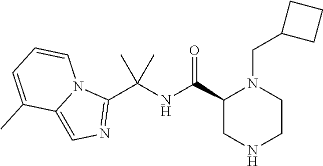 [SMILES: Cc1cccn2c(C(C)(C)NC(=O)[C@@H]3CNCCN3CC3CCC3)ncc12] | (S)-1-(cyclobutylme- thyl)-N-(2-(8-methylim- idazo[1,5-a]pyridin-3- yl)propan-2-yl)pipera- zine-2-carboxamide | INT- 091 | 370, 2.18 (I) |
| SC- 034 | 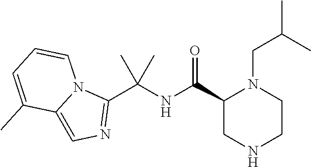 [SMILES: Cc1cccn2c(C(C)(C)NC(=O)[C@@H]3CNCCN3CC(C)C)ncc12] | (S)-1-isobutyl-N-(2-(8- methylimidazo[1,5- a]pyridin-3-yl)propan-2- yl)piperazine-2-carbox- amide | INT- 092 | 358, 2.19 (I) |
| SC- 035 | 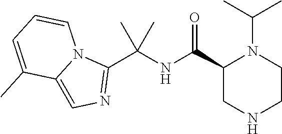 [SMILES: Cc1cccn2c(C(C)(C)NC(=O)[C@@H]3CNCCN3C(C)C)ncc12] | (S)-1-isopropyl-N-(2-(8- methylimidazo[1,5- a]pyridin-3-yl)propan-2- yl)piperazine-2-carbox- amide | INT- 080 | 344, 1.95 (I) |
| SC- 036 | 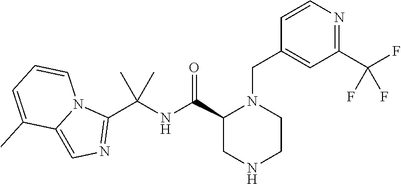 [SMILES: Cc1cccn2c(C(C)(C)NC(=O)[C@@H]3CNCCN3Cc3ccnc(C(F)(F)F)c3)ncc12] | (S)-N-(2-(8-methylimid- azo[1,5-a]pyridin-3- yl)propan-2-yl)-1-((2- (trifluoromethyl)pyridin- 4-yl)methyl)piperazine- 2-carboxamide | INT- 088 | 461, 2.18 (I) |
| SC 037 | 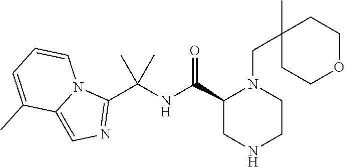 [SMILES: Cc1cccn2c(C(C)(C)NC(=O)[C@@H]3CNCCN3CC3(C)CCOCC3)ncc12] | (S)-N-(2-(8-methylimid- azo[1,5-a]pyridin-3- yl)propan-2-yl)-1-((4- methyltetrahydro-2H-py- ran-4-yl)methyl)pipera- zine-2-carboxamide | INT- 093 | 414, 2.05 (I) |
| SC- 038 | 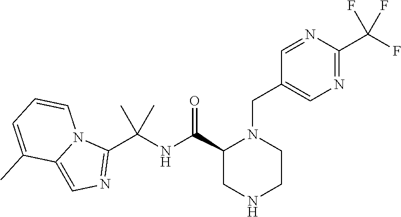 [SMILES: Cc1cccn2c(C(C)(C)NC(=O)[C@@H]3CNCCN3Cc3cnc(C(F)(F)F)nc3)ncc12] | (S)-N-(2-(8-methylimid- azo[1,5-a]pyridin-3- yl)propan-2-yl)-1-((2- (trifluoromethyl)pyrim- idin-5-yl)methyl)pipera- zine-2-carboxamide | INT- 089 | 462, 2.19 (I) |
| SC- 039 | 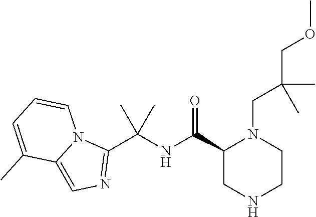 [SMILES: COCC(C)(C)CN1CCNC[C@H]1C(=O)NC(C)(C)c1ncc2c(C)cccn12] | (S)-1-(3-methoxy-2,2-di- methylpropyl)-N-(2-(8- methylimidazo[1,5- a]pyridin-3-yl)propan-2- yl)piperazine-2-carbox- amide | INT- 090 | 402, 2.27 (I) |
| SC- 040 | 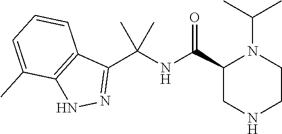 [SMILES: Cc1cccc2c(C(C)(C)NC(=O)[C@@H]3CNCCN3C(C)C)n[nH]c12] | (S)-1-isopropyl-N-(2-(7- methyl-1H-indazol-3- yl)propan-2-yl)pipera- zine-2-carboxamide | INT- 098 | 344, 1.96 (F) |
| SC- 041 | 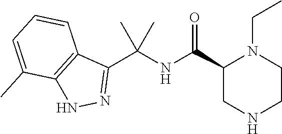 [SMILES: CCN1CCNC[C@H]1C(=O)NC(C)(C)c1n[nH]c2c(C)cccc12] | (S)-1-ethyl-N-(2-(7-me- thyl-1H-indazol-3- yl)propan-2-yl)pipera- zine-2-carboxamide | INT- 099 | 330, 1.91 (F) |

### Synthesis of SC-042: (S)—N-(2-(8-methylimidazo[1,5-a]pyridin-3-yl)propan-2-yl)-1-(oxetan-3-ylmethyl)piperazine-2-carboxamide

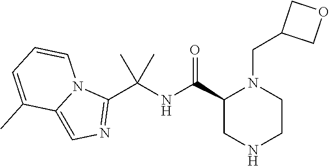 [SMILES: Cc1cccn2c(C(C)(C)NC(=O)[C@@H]3CNCCN3CC3COC3)ncc12]

A solution carbamate INT-081 (400 mg, 0.84 mmol) in hexafluoro isopropanol (5 mL) was stirred at 110° C. for 48 h. The mixture was concentrated under reduced pressure and the residue was purified by preparative HPLC (YMC-Actus Triart C18 (250×20 mm, 5μ), elution with 20 mM NH₄HCO₃ in H₂O/MeCN (80:20-5:95) at ambient temperature) to obtain the title compound (93 mg, 29%). LCMS m/z=372 [M+H]⁺; ¹H NMR (400 MHz, DMSO-d₆) δ 8.21 (s, 1H), 8.14 (d, 1H), 7.24 (s, 1H), 6.53-6.50 (m, 2H), 4.62-4.54 (m, 2H), 4.22 (t, 1H), 4.11 (t, 1H), 3.16-3.13 (m, 1H), 2.74-2.56 (m, 6H), 2.35-2.31 (m, 4H), 1.93-1.88 (m, 1H), 1.73 (s, 3H), 1.66 (s, 3H).

### Synthesis of SC-043: (S)-4-methyl-N-(2-(8-methylimidazo[1,5-a]pyridin-3-yl)propan-2-yl)piperazine-2-carboxamide

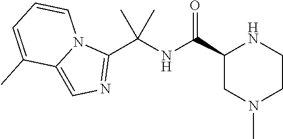 [SMILES: Cc1cccn2c(C(C)(C)NC(=O)[C@@H]3CN(C)CCN3)ncc12]

To a solution of carboxamide INT-104 (0.51 g, 1.23 mmol) in CH₂Cl₂ (18 mL) was added TFA (2.0 mL) at 0° C. and the mixture was allowed to warm to room temperature and stirred for 16 h. The mixture was concentrated under reduced pressure and the residue was purified by preparative HPLC (XBRIDGE C18 (250×19 mm, 10μ), elution with 20 mM NH₄HCO₃ in H₂O/MeOH (80:20-5:95) at ambient temperature) to afford the title compound (100 mg, 25%). LCMS m/z=316 [M+H]⁺; ¹H NMR (400 MHz, DMSO-d₆) δ 8.11 (s, 2H), 7.23 (s, 1H), 6.50 (s, 2H), 3.19 (d, 1H), 2.81 (d, 1H), 2.65-2.59 (m, 2H), 2.33 (s, 3H), 2.09 (s, 3H), 1.83-1.78 (m, 1H), 1.73-1.66 (m, 6H).

### Synthesis of SC-044: (S)-4-methyl-1-((1-methyl-1H-pyrazol-4-yl)methyl)-N-(2-(8-methylimidazo[1,5-a]pyridin-3-yl)propan-2-yl)piperazine-2-carboxamide

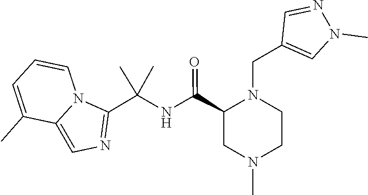

To a solution amine SC-043 (50 mg, 0.16 mmol) in CH₂Cl₂ (5 mL) was added 1-methyl-1H-pyrazole-4-carbaldehyde (44 mg, 0.40 mmol) at room temperature and the mixture was stirred for 2 h before being cooled to 0° C. NaBH(OAc)₃ (0.100 g, 0.48 mmol) was added and the mixture was allowed to warm to room temperature and stirring was continued for 16 h. The reaction was quenched by addition of aq. NaHCO₃ solution (10 mL) and the mixture was extracted with CH₂Cl₂ (2×15 mL). The combined organic layers were washed with aq. sat. NaCl solution (15 mL), dried over anhyd. Na₂SO₄, filtered and concentrated under reduced pressure. The residue was purified by preparative HPLC (YMC-Actus Triart C18 (250×20 mm, 5μ), elution with 20 mM NH₄HCO₃ in H₂O/MeCN (80:20-5:95) at ambient temperature) to obtain the title compound (11 mg, 65%). LCMS m/z=410 [M+H]⁺; ¹H NMR (400 MHz, DMSO-d₆) δ 8.32 (s, 1H), 8.13 (d, 1H), 7.52 (s, 1H), 7.28 (d, 2H), 6.46-6.49 (m, 2H), 3.78 (s, 3H), 3.49 (d, 1H), 3.13 (d, 1H), 2.80-2.69 (m, 2H), 2.57-2.54 (m, 2H), 2.34 (s, 3H), 2.11-2.05 (m, 6H), 1.73 (s, 3H), 1.71 (s, 3H).

### Synthesis of SC-045: (S)-4-methyl-N-(2-(8-methylimidazo[1,5-a]pyridin-3-yl)propan-2-yl)-1-(2,2,2-trifluoroethyl)piperazine-2-carboxamide

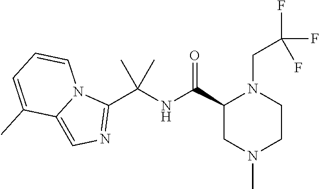 [SMILES: Cc1cccn2c(C(C)(C)NC(=O)[C@@H]3CN(C)CCN3CC(F)(F)F)ncc12]

To a solution of carboxamide SC-043 (0.110 g, 0.16 mmol) in MeCN (6 mL) were added K₂CO₃ (145 mg, 1.05 mmol) and 2,2,2-trifluoroethyl trifluoromethanesulfonate (0.122 g, 0.52 mmol) at room temperature and the mixture was stirred for 48 h. The mixture was extracted with CH₂Cl₂ (2×15 mL) and the combined organic layers were washed with aq. sat. NaCl solution (15 mL), dried over anhyd. Na₂SO₄, filtered and concentrated under reduced pressure. The residue was purified by preparative HPLC (YMC-Actus Triart C18 (250×20 mm, 5μ), elution with 20 mM NH₄HCO₃ in H₂O/MeCN (80:20-5:95) at ambient temperature) to afford partially racemized title product. The enantiomers were separated by preparative chiral HPLC (Chiralart Cellulose SC (250×20 mm, 5μ), elution with isocratic CH₂Cl₂/EtOH 90:10 at ambient temperature) to afford the title compound (15 mg, 10%). LCMS m/z=399 [M+H]⁺; ¹H NMR (400 MHz, DMSO-d₆, 79° C.) δ 8.18 (s, 1H), 8.10 (s, 1H), 7.25 (s, 1H), 6.50 (s, 2H), 3.30 (s, 1H), 2.67 (s, 2H), 2.45 (m, 1H), 2.35 (m, 5H), 2.18 (s, 3H), 1.71 (s, 6H). Chiral LC: Colum Chiralpak IC (4.6×250 mm, 5μ); elution with hexane/CH₂Cl₂/EtOH/iPrNH₂ (60:20:20:0.1) at 1.0 mL/min; (S)-enantiomer Rₜ=5.75 min.

### Synthesis of SC-046: (S)-1,4-dimethyl-N-(2-(8-methylimidazo[1,5-a]pyridin-3-yl)propan-2-yl)piperazine-2-carboxamide

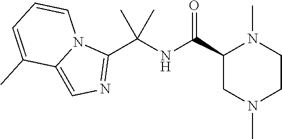 [SMILES: Cc1cccn2c(C(C)(C)NC(=O)[C@@H]3CN(C)CCN3C)ncc12]

To a solution of carboxamide SC-043 (80 m, 0.25 mmol) in MeOH (5 mL) were added formaldehyde (aq. 37% solution, 0.037 mL, 0.37 mmol), NaOAc (62 mg, 0.76 mmol) and AcOH (0.05 mL) at room temperature and the mixture was stirred for 2 h. The mixture was cooled to 0° C. and NaBH₃CN (47 mg, 0.76 mmol) was added. The mixture was allowed to warm to room temperature and stirring was continued for 16 h. The reaction was quenched by addition of aq. NaHCO₃ solution (10 mL) and the mixture was extracted with CH₂Cl₂ (2×15 mL). The combined organic layers were washed with aq. sat. NaCl solution (15 mL), dried over anhyd. Na₂SO₄, filtered and concentrated under reduced pressure. The residue was purified by preparative HPLC (YMC-Actus Triart C18 (250×20 mm, 5μ), elution with 20 mM NH₄HCO₃ in H₂O/MeCN (80:20-5:95) at ambient temperature) to afford the title compound (30 mg, 35%). LCMS m/z=330 [M+H]⁺; ¹H NMR (400 MHz, DMSO-d₆) δ 8.17 (s, 1H), 8.12 (d, 1H), 7.24 (s, 1H), 6.53-6.50 (m, 2H), 2.73-2.70 (m, 1H), 2.59-2.55 (m, 4H), 2.33 (s, 3H), 2.12-2.10 (m, 4H), 2.05 (s, 4H), 2.02-1.93 (m, 1H), 1.70 (s, 3H), 1.66 (s, 3H).

### Synthesis of SC-047: (S)-4-methyl-N-(2-(8-methylimidazo[1,5-a]pyridin-3-yl)propan-2-yl)-1-((tetrahydro-2H-pyran-4-yl)methyl)piperazine-2-carboxamide

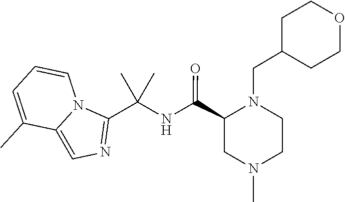 [SMILES: Cc1cccn2c(C(C)(C)NC(=O)[C@@H]3CN(C)CCN3CC3CCOCC3)ncc12]

To a solution of carboxamide SC-043 (70 mg, 0.22 mmol) in CH₂Cl₂ (3 mL) were added tetrahydro-2H-pyran-4-carbaldehyde (64 mg, 0.56 mmol) and NaOAc (55 mg, 0.67 mmol) at room temperature and the mixture was stirred for 2 h. The mixture was cooled to 0° C. and NaBH(OAc)₃ (0.142 g, 0.67 mmol) was added. The mixture was allowed to warm to room temperature and stirring was continued for 16 h. The mixture was quenched by addition of aq. NaHCO₃ solution of (10 mL) and extracted with CH₂Cl₂ (2×15 mL). The combined organic layers were washed with aq. sat. NaCl solution (15 mL), dried over anhyd. Na₂SO₄, filtered and concentrated under reduced pressure. The residue was purified by preparative HPLC (XBRIDGE C18 (250×19 mm, 10μ), elution with 20 mM NH₄HCO₃ in H₂O/MeOH (80:20-5:95) at ambient temperature) to obtain the title compound (20 mg, 21%). LCMS m/z=414 [M+H]⁺; ¹H NMR (400 MHz, DMSO-d₆) δ 8.15-8.12 (m, 2H), 7.24 (s, 1H), 6.52-6.50 (m, 2H), 3.76-3.73 (m, 2H), 3.23-3.17 (m, 2H), 2.92-2.90 (m, 1H), 2.75-2.73 (m, 1H), 2.33 (s, 3H), 2.19-2.05 (m, 7H), 1.96-1.93 (m, 1H), 1.73-1.66 (m, 9H), 1.43 (d, 1H), 0.99-0.91 (m, 2H).

The following compound was prepared in analogy:

| | | | | LC-MS m/z |
|---|---|---|---|---|
| Ex- | | | | [M + H]⁺, Rt |
| am- | | | | [min] (Me- |
| ple | Structure | Chemical Name | Precursor | thod) |
| SC- 048 | 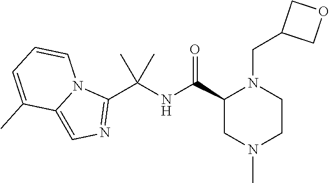 [SMILES: Cc1cccn2c(C(C)(C)NC(=O)[C@@H]3CN(C)CCN3CC3COC3)ncc12] | (S)-4-methyl-N-(2-(8-me- thylimidazo[1,5-a]pyridin- 3-yl)propan-2-yl)-1-(ox- etan-3-ylmethyl)pipera- zine-2-carboxamide | SC-043 and oxetane-3- carbalde- hyde | 386, 1.89 (I) |

### Synthesis of SC-049: (S)—N-(2-(8-methylimidazo[1,5-a]pyridin-3-yl)propan-2-yl)-4-(2,2,2-trifluoroethyl)piperazine-2-carboxamide

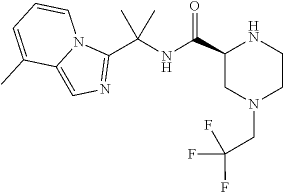 [SMILES: Cc1cccn2c(C(C)(C)NC(=O)[C@@H]3CN(CC(F)(F)F)CCN3)ncc12]

To a solution of carboxamide INT-102 (100 mg, 0.25 mmol) in CH₂Cl₂ (3 mL) were added 2,2,2-trifluoroethyl trifluoromethanesulfonate (58 mg, 0.25 mmol) and Et₃N (38 mg, 0.37 mmol) at room temperature under nitrogen atmosphere. The reaction mixture was stirred for 12 h at the same temperature before TFA (1 mL) was added to the solution. The mixture was stirred for 12 h at room temperature and was subsequently concentrated under reduced pressure. The residue was purified by preparative HPLC (Phenomenex C18 (100×40 mm, 3μ), elution with 0.1% TFA in H₂O/MeCN (90:10-48:52) to obtain the title compound (74 mg, 78%) as its TFA salt. LCMS m/z=384 [M+H]⁺; ¹H NMR (400 MHz, DMSO-d₆) δ 9.38 (s, 1H), 9.23 (br, 1H), 8.74 (s, 1H), 8.16 (d, 1H), 7.73 (s, 1H), 6.85-6.72 (m, 2H), 4.02 (s, 1H), 3.50-3.30 (m, 3H), 3.14-2.98 (m, 2H), 2.88 (d, 1H), 2.72-2.63 (m, 1H), 2.58 (t, 1H), 2.43-2.36 (m, 3H), 1.80 (s, 3H), 1.75 (s, 3H).

### Synthesis of SC-050: (S)-4-(2,2-difluoroethyl)-N-(2-(8-methylimidazo[1,5-a]pyridin-3-yl)propan-2-yl)piperazine-2-carboxamide

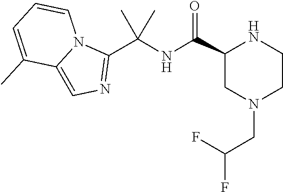 [SMILES: Cc1cccn2c(C(C)(C)NC(=O)[C@@H]3CN(CC(F)F)CCN3)ncc12]

To a solution of carboxamide INT-102 (100 mg, 0.25 mmol) in THF (3 mL) were added KI (124 mg, 0.75 mmol), DIPEA (120 μL, 0.75 mmol) and 2-bromo-1,1-difluoroethane (72 mg, 0.25 mmol) at room temperature under nitrogen atmosphere. The mixture was stored for 72 h at 60° C. before it was allowed to cool and concentrated under reduced pressure. The residue was dissolved in CH₂Cl₂ (3 mL) and TFA (1 mL) was added at room temperature and the mixture stirred for 12 h. The mixture was concentrated under reduced pressure and the residue was purified by preparative HPLC (Phenomenex C18 (100×40 mm, 3μ), elution with 0.1% TFA in H₂O/MeCN (90:10-48:52) to obtain the title compound (14 mg, 15%) as its TFA salt. LCMS m/z=366 [M+H]⁺; ¹H NMR (400 MHz, DMSO-d₆) δ 8.15-8.10 (m, 2H), 7.23 (s, 1H), 6.54-6.48 (m, 2H), 6.20 (tt, 1H), 3.22 (s, 1H), 2.82 (d, 2H), 2.70-2.62 (m, 4H), 2.33 (s, 3H), 2.23-2.06 (m, 2H), 1.99 (t, 1H), 1.69 (s, 3H), 1.67 (s, 3H).

The following compound was prepared in analogy:

| | | | | LC-MS m/z |
|---|---|---|---|---|
| Ex- | | | | [M + H]⁺, Rt |
| am- | | | | [min] (Me- |
| ple | Structure | Chemical Name | Precursor | thod) |
| SC- 051 | 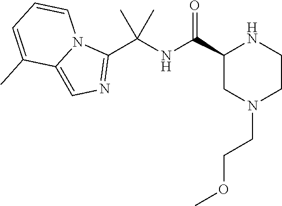 [SMILES: COCCN1CCN[C@H](C(=O)NC(C)(C)c2ncc3c(C)cccn23)C1] | (S)-4-(2-methoxyethyl)- N-(2-(8-methylimid- azo[1,5-a]pyridin-3- yl)propan-2-yl)piperazine- 2-carboxamide | INT-102 and 1-chloro-2- methoxye- thane | 360, 2.14 (L) |

### Synthesis of SC-052: (S)-4-isopropyl-N-(2-(8-methylimidazo[1,5-a]pyridin-3-yl)propan-2-yl)piperazine-2-carboxamide

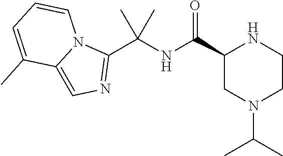 [SMILES: Cc1cccn2c(C(C)(C)NC(=O)[C@@H]3CN(C(C)C)CCN3)ncc12]

To a solution of carboxamide INT-102 (100 mg, 0.25 mmol) and acetone (15 mg, 0.25 mmol) in MeOH (3 mL) were added NaBH₃CN (16 mg, 0.25 mmol) and trimethylamine (30% in EtOH, 0.1 mL, 0.5 mmol) at room temperature under nitrogen atmosphere. The mixture was stirred for 1 h before AcOH (15 mg, 0.25 mmol) was added and stirring was continued for 72 h. The mixture was concentrated under reduced pressure and the residue was dissolved in CH₂Cl₂ (3 mL) and TFA (1 mL) was added. The mixture was stirred for 12 h at room temperature and subsequently concentrated under reduced pressure. The residue was purified by preparative HPLC (Phenomenex C18 (80×40 mm, 3μ), elution with 10 mM NH₄HCO₃ in H₂O/MeCN (85:15-55:45) to obtain the title product (49 mg, 57%) as its TFA salt. LCMS m/z=344 [M+H]⁺; ¹H NMR (400 MHz, DMSO-d₆) δ 8.25-8.12 (m, 2H), 7.30 (s, 1H), 6.61-6.51 (m, 2H), 3.24-3.19 (m, 1H), 2.90 (d, 1H), 2.78-2.72 (m, 1H), 2.70-2.58 (m, 3H), 2.40 (s, 3H), 2.25 (br, 1H), 2.15-2.07 (m, 1H), 1.99 (t, 1H), 1.75 (s, 3H), 1.73 (s, 3H), 1.02-0.95 (m, 6H) 356.

The following compounds were prepared in analogy:

| | | | | LC-MS m/z |
|---|---|---|---|---|
| Ex- | | | | [M + H]⁺, Rt |
| am- | | | | [min] (Me- |
| ple | Structure | Chemical Name | Precursor | thod) |
| SC- 053 | 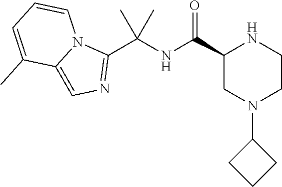 [SMILES: Cc1cccn2c(C(C)(C)NC(=O)[C@@H]3CN(C4CCC4)CCN3)ncc12] | (S)-4-cyclobutyl-N-(2-(8- methylimidazo[1,5-a]pyri- din-3-yl)propan-2-yl)pi- perazine-2-carboxamide | INT-102 and cyclobutanone | 356, 1.36 (L) |
| SC- 054 | 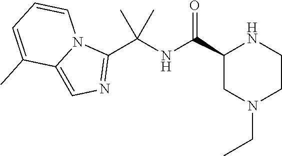 [SMILES: CCN1CCN[C@H](C(=O)NC(C)(C)c2ncc3c(C)cccn23)C1] | (S)-4-ethyl-N-(2-(8-me- thylimidazo[1,5-a]pyridin- 3-yl)propan-2-yl)pipera- zine-2-carboxamide | INT-102 and acetaldehyde | 330, 1.54 (L) |
| SC- 055 | 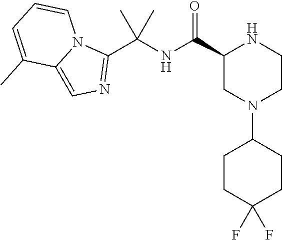 [SMILES: Cc1cccn2c(C(C)(C)NC(=O)[C@@H]3CN(C4CCC(F)(F)CC4)CCN3)ncc12] | (S)-4-(4,4-difluorocyclo- hexyl)-N-(2-(8-methylim- idazo[1,5-a]pyridin-3- yl)propan-2-yl)piperazine- 2-carboxamide | INT-102 and 4,4-difluorocy- clohexan-1-one | 420, 2.51 (L) |
| SC- 056 | 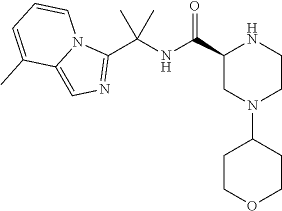 [SMILES: Cc1cccn2c(C(C)(C)NC(=O)[C@@H]3CN(C4CCOCC4)CCN3)ncc12] | (S)-N-(2-(8-methylimid- azo[1,5-a]pyridin-3- yl)propan-2-yl)-4-(tetrahy- dro-2H-pyran-4-yl)pipera- zine-2-carboxamide | INT-102 and tetrahydro-4H- pyran-4-one | 386, 1.25 (L) |
| SC- 057 | 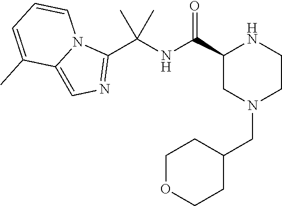 [SMILES: Cc1cccn2c(C(C)(C)NC(=O)[C@@H]3CN(CC4CCOCC4)CCN3)ncc12] | (S)-N-(2-(8-methylimid- azo[1,5-a]pyridin-3- yl)propan-2-yl)-4-((tetra- hydro-2H-pyran-4-yl)me- thyl)piperazine-2-carbox- amide | INT-102 and tetrahydro-2H- pyran-4-carbal- dehyde | 400, 2.26 (L) |
| SC- 058 | 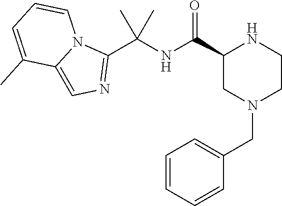 [SMILES: Cc1cccn2c(C(C)(C)NC(=O)[C@@H]3CN(Cc4ccccc4)CCN3)ncc12] | (S)-4-benzyl-N-(2-(8-me- thylimidazo[1,5-a]pyridin- 3-yl)propan-2-yl)pipera- zine-2-carboxamide | INT-102 and benzaldehyde | 392, 1.58 (L) |
| SC- 059 | 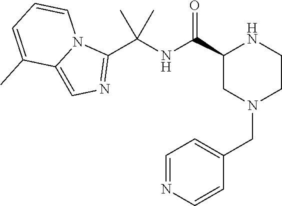 [SMILES: Cc1cccn2c(C(C)(C)NC(=O)[C@@H]3CN(Cc4ccncc4)CCN3)ncc12] | (S)-N-(2-(8-methylimid- azo[1,5-a]pyridin-3- yl)propan-2-yl)-4-(pyri- din-4-ylmethyl)pipera- zine-2-carboxamide | INT-102 and isonicotinalde- hyde | 393, 1.74 (L) |
| SC- 060 | 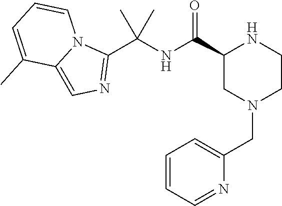 [SMILES: Cc1cccn2c(C(C)(C)NC(=O)[C@@H]3CN(Cc4ccccn4)CCN3)ncc12] | (S)-N-(2-(8-methylimid- azo[1,5-a]pyridin-3- yl)propan-2-yl)-4-(pyri- din-2-ylmethyl)pipera- zine-2-carboxamide | INT-102 and picolinalde- hyde | 393, 1.74 (L) |
| SC- 061 | 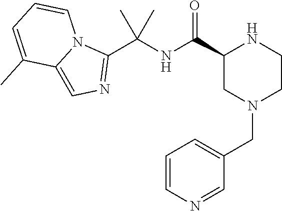 [SMILES: Cc1cccn2c(C(C)(C)NC(=O)[C@@H]3CN(Cc4cccnc4)CCN3)ncc12] | (S)-N-(2-(8-methylimid- azo[1,5-a]pyridin-3- yl)propan-2-yl)-4-(pyri- din-3-ylmethyl)pipera- zine-2-carboxamide | INT-102 and nicotinalde- hyde | 393, 1.77 (L) |
| SC- 062 | 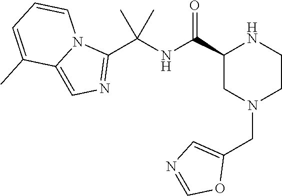 [SMILES: Cc1cccn2c(C(C)(C)NC(=O)[C@@H]3CN(Cc4cnco4)CCN3)ncc12] | (S)-N-(2-(8-methylimid- azo[1,5-a]pyridin-3- yl)propan-2-yl)-4-(oxazol- 5-ylmethyl)piperazine-2- carboxamide | INT-102 and oxazole-5- carbaldehyde | 383, 2.06 (L) |
| SC- 063 | 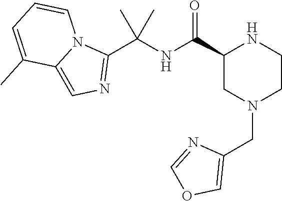 [SMILES: Cc1cccn2c(C(C)(C)NC(=O)[C@@H]3CN(Cc4cocn4)CCN3)ncc12] | (S)-N-(2-(8-methylimid- azo[1,5-a]pyridin-3- yl)propan-2-yl)-4-(oxazol- 4-ylmethyl)piperazine-2- carboxamide | INT-102 and oxazole-4- carbaldehyde | 383, 2.03 (L) |
| SC- 064 | 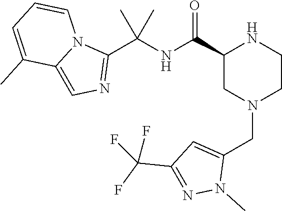 [SMILES: Cc1cccn2c(C(C)(C)NC(=O)[C@@H]3CN(Cc4cc(C(F)(F)F)nn4C)CCN3)ncc12] | (S)-4-((1-methyl-3-(tri- fluoromethyl)-1H-pyrazol- 5-yl)methyl)-N-(2-(8-me- thylimidazo[1,5-a]pyridin- 3-yl)propan-2-yl)pipera- zine-2-carboxamide | INT-102 and 1-methyl-3- (trifluorome- thyl)-1H-pyra- zole-5-carbal- dehyde | 464, 2.57 (L) |
| SC- 065 | 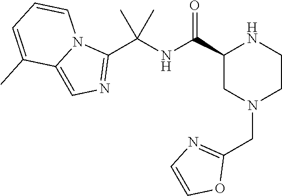 [SMILES: Cc1cccn2c(C(C)(C)NC(=O)[C@@H]3CN(Cc4ncco4)CCN3)ncc12] | (S)-N-(2-(8-methylimid- azo[1,5-a]pyridin-3- yl)propan-2-yl)-4-(oxazol- 2-ylmethyl)piperazine-2- carboxamide | INT-102 and oxazole-2- carbaldehyde | 383, 1.50 (L) |
| SC- 066 | 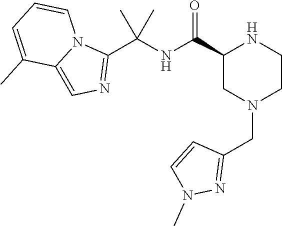 [SMILES: Cc1cccn2c(C(C)(C)NC(=O)[C@@H]3CN(Cc4ccn(C)n4)CCN3)ncc12] | (S)-4-((1-methyl-1H-pyra- zol-3-yl)methyl)-N-(2-(8- methylimidazo[1,5-a]pyri- din-3-yl)propan-2-yl)pi- perazine-2-carboxamide | INT-102 and 1-methyl-1H- pyrazole-3- carbaldehyde | 396, 1.59 (L) |
| SC- 067 | 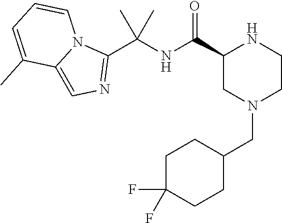 [SMILES: Cc1cccn2c(C(C)(C)NC(=O)[C@@H]3CN(CC4CCC(F)(F)CC4)CCN3)ncc12] | (S)-4-((4,4-difluorocyclo- hexyl)methyl)-N-(2-(8- methylimidazo[1,5-a]pyri- din-3-yl)propan-2-yl)pi- perazine-2-carboxamide | INT-102 and 4,4-difluorocy- clohexane-1- carbaldehyde | 434, 2.68 (L) |
| SC- 068 | 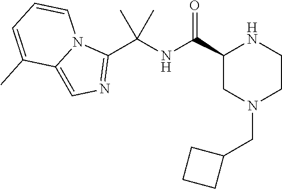 [SMILES: Cc1cccn2c(C(C)(C)NC(=O)[C@@H]3CN(CC4CCC4)CCN3)ncc12] | (S)-4-(cyclobutylmethyl)- N-(2-(8-methylimid- azo[1,5-a]pyridin-3- yl)propan-2-yl)piperazine- 2-carboxamide | INT-102 and cyclobutane- carbaldehyde | 370, 2.39 (L) |
| SC- 069 | 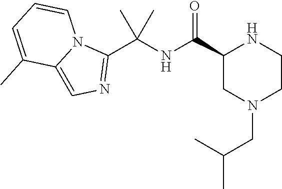 [SMILES: Cc1cccn2c(C(C)(C)NC(=O)[C@@H]3CN(CC(C)C)CCN3)ncc12] | (S)-4-isobutyl-N-(2-(8- methylimidazo[1,5-a]pyri- din-3-yl)propan-2-yl)pi- perazine-2-carboxamide | INT-102 and isobutyralde- hyde | 358, 2.39 (L) |
| SC- 070 | 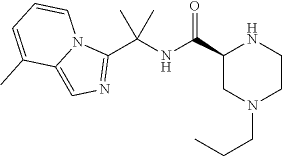 [SMILES: CCCN1CCN[C@H](C(=O)NC(C)(C)c2ncc3c(C)cccn23)C1] | (S)-N-(2-(8-methylimid- azo[1,5-a]pyridin-3- yl)propan-2-yl)-4- propylpiperazine-2-car- boxamide | INT-102 and propionalde- hyde | 344, 1.97 (L) |
| SC- 071 | 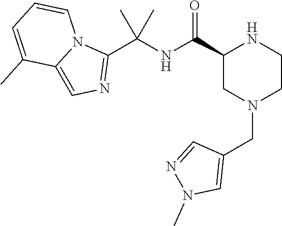 [SMILES: Cc1cccn2c(C(C)(C)NC(=O)[C@@H]3CN(Cc4cnn(C)c4)CCN3)ncc12] | (S)-4-((1-methyl-1H-pyra- zol-4-yl)methyl)-N-(2-(8- methylimidazo[1,5-a]pyri- din-3-yl)propan-2-yl)pi- perazine-2-carboxamide | INT-102 and 1-methyl-1H- pyrazole-4- carbaldehyde | 396, 1.52 (L) |
| SC- 072 | 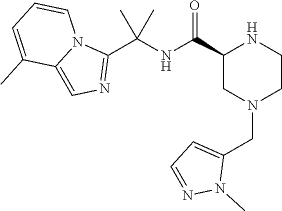 [SMILES: Cc1cccn2c(C(C)(C)NC(=O)[C@@H]3CN(Cc4ccnn4C)CCN3)ncc12] | (S)-4-((1-methyl-1H-pyra- zol-5-yl)methyl)-N-(2-(8- methylimidazo[1,5-a]pyri- din-3-yl)propan-2-yl)pi- perazine-2-carboxamide | INT-102 and 1-methyl-1H- pyrazole-5- carbaldehyde | 396, 1.52 (L) |
| SC- 073 | 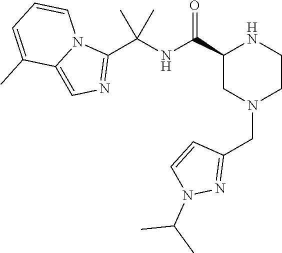 [SMILES: Cc1cccn2c(C(C)(C)NC(=O)[C@@H]3CN(Cc4ccn(C(C)C)n4)CCN3)ncc12] | (S)-4-((1-isopropyl-1H- pyrazol-3-yl)methyl)-N- (2-(8-methylimidazo[1,5- a]pyridin-3-yl)propan-2- yl)piperazine-2-carbox- amide | INT-102 and 1-isopropyl- 1H-pyrazole-3- carbaldehyde | 424, 1.91 (L) |
| SC- 074 | 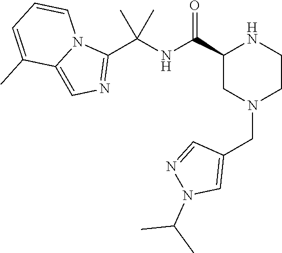 [SMILES: Cc1cccn2c(C(C)(C)NC(=O)[C@@H]3CN(Cc4cnn(C(C)C)c4)CCN3)ncc12] | (S)-4-((1-isopropyl-1H- pyrazol-4-yl)methyl)-N- (2-(8-methylimidazo[1,5- a]pyridin-3-yl)propan-2- yl)piperazine-2-carbox- amide | INT-102 and 1-isopropyl- 1H-pyrazole-4- carbaldehyde | 424, 1.90 (L) |

### Synthesis of SC-075: (S)—N-(2-(8-methylimidazo[1,5-a]pyridin-3-yl)propan-2-yl)-4-(oxetan-3-yl)piperazine-2-carboxamide

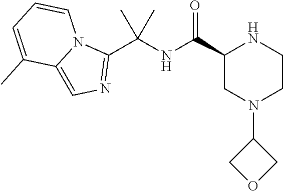 [SMILES: Cc1cccn2c(C(C)(C)NC(=O)[C@@H]3CN(C4COC4)CCN3)ncc12]

To a solution of carboxamide INT-102 (100 mg, 0.25 mmol) and oxetan-3-one (18 mg, 0.25 mmol) in MeOH (3 mL) were added NaBH₃CN (16 mg, 0.25 mmol) and trimethylamine (30% in EtOH, 0.1 mL, 0.5 mmol) at room temperature under nitrogen atmosphere. The mixture was stirred for 1 h before AcOH (15 mg, 0.25 mmol) was added and stirring was continued for 72 h. The mixture was concentrated under reduced pressure and the residue was dissolved in H₂O (3 mL) and stirred at 100° C. for 12 h. The mixture was allowed to cool to room temperature and subsequently concentrated under reduced pressure. The residue was purified by preparative HPLC (Phenomenex C18 (80×40 mm, 3μ), elution with 10 mM NH₄HCO₃ in H₂O/MeCN (90:10-60:40) to obtain the title product (24 mg, 27%). LCMS m/z=358 [M+H]⁺; ¹H NMR (400 MHz, DMSO-d₆) δ 8.20-8.12 (m, 2H), 7.32-7.17 (m, 1H), 6.60-6.38 (m, 2H), 4.62-4.20 (m, 2H), 3.60 (t, 1H), 3.49-3.42 (m, 1H), 3.26-3.12 (m, 2H), 2.89-2.56 (m, 5H), 2.45 (d, 1H), 2.33 (s, 3H), 2.27-2.08 (m, 1H), 1.78-1.70 (m, 3H), 1.70-1.64 (m, 3H).

The following compound was prepared in analogy:

| | | | | LC-MS m/z |
|---|---|---|---|---|
| Ex- | | | | [M + H]⁺, Rt |
| am- | | | | [min] (Me- |
| ple | Structure | Chemical Name | Precursor | thod) |
| SC- 076 | 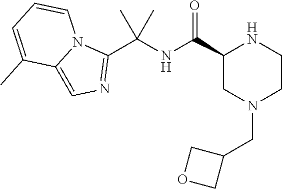 [SMILES: Cc1cccn2c(C(C)(C)NC(=O)[C@@H]3CN(CC4COC4)CCN3)ncc12] | (S)-N-(2-(8-methylimid- azo[1,5-a]pyridin-3- yl)propan-2-yl)-4-(oxetan- 3-ylmethyl)piperazine-2- carboxamide | INT-102 and ox- etane-3- carbalde- hyde | 372, 2.04 (L) |

### Synthesis of SC-077: (S)-4-(cyclopropanecarbonyl)-N-(2-(8-methylimidazo[1,5-a]pyridin-3-yl)propan-2-yl)piperazine-2-carboxamide

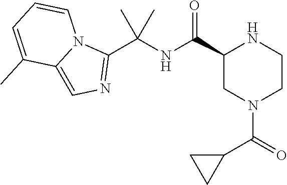 [SMILES: Cc1cccn2c(C(C)(C)NC(=O)[C@@H]3CN(C(=O)C4CC4)CCN3)ncc12]

To a solution of cyclopropanecarboxylic acid (22 mg, 0.25 mmol) in DMF (3 mL) were added HATU (95 mg, 0.25 mmol) and DIPEA (64 mg, 0.5 mmol) at room temperature under nitrogen atmosphere. The mixture was stirred for 1 h at that temperature before carboxamide INT-102 (100 mg, 0.25 mmol) was added and the mixture continued to stir for 72 h. The mixture was concentrated under reduced pressure and the residue was dissolved in CH₂Cl₂ (3 mL). TFA (1 mL) was added and the mixture was stirred for 12 h at room temperature before being concentrated under reduced pressure. The residue was purified by preparative HPLC (Phenomenex C18 (80×40 mm, 3μ), elution with 10 mM NH₄HCO₃ in H₂O/MeCN (85:15-55:45) at ambient temperature) to obtain the title compound (55 mg, 60%) as its TFA salt. LCMS m/z=370 [M+H]⁺; ¹H NMR (400 MHz, DMSO-d₆) δ 8.26 (s, 1H), 8.13 (s, 1H), 7.25 (s, 1H), 6.50 (m, 2H), 4.32-3.70 (m, 2H), 3.28-3.03 (m, 2H), 2.96-2.54 (m, 3H), 2.34 (s, 3H), 1.92 (s, 1H), 1.68 (s, 6H), 0.70 (s, 4H).

The following compounds were prepared in analogy:

| | | | | LC-MS m/z |
|---|---|---|---|---|
| | | | | [M + H]⁺, Rt |
| Exam- | | | | [min] (Me- |
| ple | Structure | Chemical Name | Precursor | thod) |
| SC- 078 | 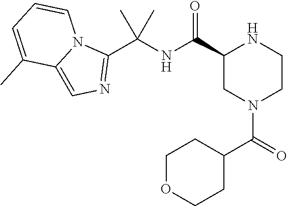 [SMILES: Cc1cccn2c(C(C)(C)NC(=O)[C@@H]3CN(C(=O)C4CCOCC4)CCN3)ncc12] | (S)-N-(2-(8-methylimid- azo[1,5-a]pyridin-3-yl)pro- pan-2-yl)-4-(tetrahydro- 2H-pyran-4-carbonyl)pi- perazine-2-carboxamide | INT-102 and tetrahy- dro-2H-py- ran-4-car- boxylic acid | 414, 2.09 (L) |
| SC- 079 | 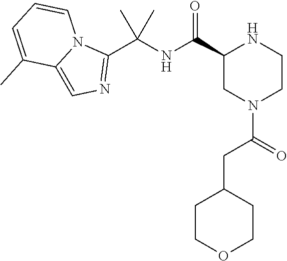 [SMILES: Cc1cccn2c(C(C)(C)NC(=O)[C@@H]3CN(C(=O)CC4CCOCC4)CCN3)ncc12] | (S)-N-(2-(8-methylimid- azo[1,5-a]pyridin-3-yl)pro- pan-2-yl)-4-(2-(tetrahydro- 2H-pyran-4-yl)acetyl)pi- perazine-2-carboxamide | INT-102 and 2-(tet- rahydro- 2H-pyran- 4-yl)acetic acid | 428, 2.14 (L) |
| SC- 080 | 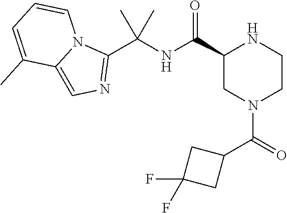 [SMILES: Cc1cccn2c(C(C)(C)NC(=O)[C@@H]3CN(C(=O)C4CC(F)(F)C4)CCN3)ncc12] | (S)-4-(3,3-difluorocyclo- butane-1-carbonyl)-N-(2- (8-methylimidazo[1,5- a]pyridin-3-yl)propan-2- yl)piperazine-2-carbox- amide | INT-102 and 3,3- difluorocy- clobutane- 1-carbox- ylic acid | 420, 1.97 (L) |
| SC- 081 | 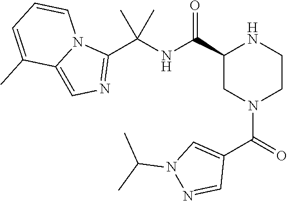 [SMILES: Cc1cccn2c(C(C)(C)NC(=O)[C@@H]3CN(C(=O)c4cnn(C(C)C)c4)CCN3)ncc12] | (S)-4-(1-isopropyl-1H-py- razole-4-carbonyl)-N-(2- (8-methylimidazo[1,5- a]pyridin-3-yl)propan-2- yl)piperazine-2-carbox- amide | INT-102 and 1-iso- propyl-1H- pyrazole-4- carboxylic acid | 438, 1.78 (L) |
| SC- 082 | 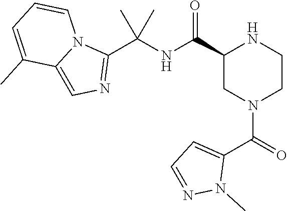 [SMILES: Cc1cccn2c(C(C)(C)NC(=O)[C@@H]3CN(C(=O)c4ccnn4C)CCN3)ncc12] | (S)-4-(1-methyl-1H-pyra- zole-5-carbonyl)-N-(2-(8- methylimidazo[1,5-a]pyri- din-3-yl)propan-2-yl)pi- perazine-2-carboxamide | INT-102 and 1-me- thyl-1H-py- razole-5- carboxylic acid | 410, 1.52 (L) |
| SC- 083 | 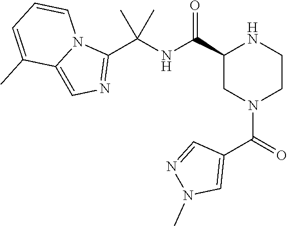 [SMILES: Cc1cccn2c(C(C)(C)NC(=O)[C@@H]3CN(C(=O)c4cnn(C)c4)CCN3)ncc12] | (S)-4-(1-methyl-1H-pyra- zole-4-carbonyl)-N-(2-(8- methylimidazo[1,5-a]pyri- din-3-yl)propan-2-yl)pi- perazine-2-carboxamide | INT-102 and 1-me- thyl-1H-py- razole-4- carboxylic acid | 410, 1.39 (L) |
| SC- 084 | 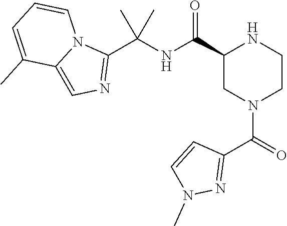 [SMILES: Cc1cccn2c(C(C)(C)NC(=O)[C@@H]3CN(C(=O)c4ccn(C)n4)CCN3)ncc12] | (S)-4-(1-methyl-1H-pyra- zole-3-carbonyl)-N-(2-(8- methylimidazo[1,5-a]pyri- din-3-yl)propan-2-yl)pi- perazine-2-carboxamide | INT-102 and 1-me- thyl-1H-py- razole-3- carboxylic acid | 410, 1.42 (L) |

### Synthesis of SC-085: (S)-1-benzyl-N-(2-(8-methylimidazo[1,5-a]pyridin-3-yl)propan-2-yl)piperazine-2-carboxamide

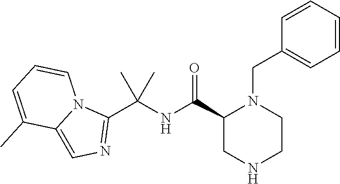 [SMILES: Cc1cccn2c(C(C)(C)NC(=O)[C@@H]3CNCCN3Cc3ccccc3)ncc12]

To a solution carboxamide INT-103 (160 mg, 0.33 mmol) in CH₂Cl₂ (10 mL) was added TFA (3.0 mL) at 0° C. and stirred at room temperature for 3 h. The mixture was concentrated under reduced pressure, diluted with MeOH (5 mL) and stirred with amberlyst-A21 resin (500 mg) for 2 h. The mixture was filtered, washed with MeOH and concentrated under reduced pressure. The residue was purified by preparative HPLC (Lymc-SG column, 20 mM NH₄CO₃ in H₂O/MeCN 80:20-5:95, ambient temperature) to obtain the title compound (50 mg, 39%). LCMS m/z=392 [M+H]⁺; ¹H NMR (400 MHz, DMSO-d₆, 100° C.) δ 8.11-8.10 (m, 2H), 7.45-7.22 (m, 6H), 6.48-6.46 (m, 1H), 6.42-6.39 (m, 1H), 3.77-3.71 (m, 1H), 3.29-3.20 (m, 1H), 2.96-2.91 (m, 2H), 2.88-2.81 (m, 2H), 2.78-2.63 (m, 4H), 2.35 (s, 3H), 2.10-2.04 (m, 1H), 1.76-1.75 (m, 6H).

The following compounds were prepared in analogy:

| | | | | LC-MS |
|---|---|---|---|---|
| | | | | m/z |
| Ex- | | | Pre- | [M + H]⁺, |
| am- | | | cur- | Rt [min] |
| ple | Structure | Chemical Name | sor | (Method) |
| SC- 086 | 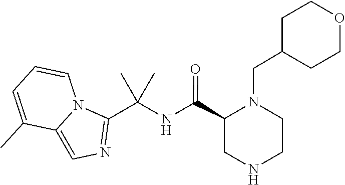 [SMILES: Cc1cccn2c(C(C)(C)NC(=O)[C@@H]3CNCCN3CC3CCOCC3)ncc12] | (S)-N-(2-(8-methylimid- azo[1,5-a]pyridin-3-yl)pro- pan-2-yl)-1-((tetrahydro- 2H-pyran-4-yl)methyl)pi- perazine-2-carboxamide | INT- 105 | 400, 1.95 (I) |
| SC- 087 | 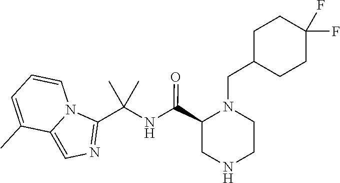 [SMILES: Cc1cccn2c(C(C)(C)NC(=O)[C@@H]3CNCCN3CC3CCC(F)(F)CC3)ncc12] | (S)-1-((4,4-difluorocyclo- hexyl)methyl)-N-(2-(8-me- thylimidazo[1,5-a]pyridin-3- yl)propan-2-yl)piperazine-2- carboxamide | INT- 106 | 432 [M − H]⁻, 2.27 (I) |
| SC- 088 | 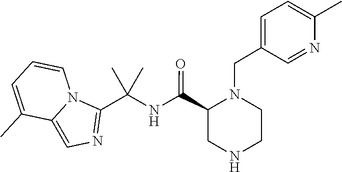 | (S)-N-(2-(8-methylimid- azo[1,5-a]pyridin-3-yl)pro- pan-2-yl)-1-((6-methylpyri- din-3-yl)methyl)piperazine- 2-carboxamide | INT- 107 | 407, 2.00 (I) |
| SC- 089 | 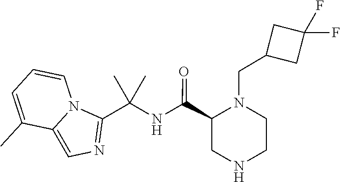 [SMILES: Cc1cccn2c(C(C)(C)NC(=O)[C@@H]3CNCCN3CC3CC(F)(F)C3)ncc12] | (S)-1-((3,3-difluorocyclobu- tyl)methyl)-N-(2-(8-me- thylimidazo[1,5-a]pyridin-3- yl)propan-2-yl)piperazine-2- carboxamide | INT- 108 | 406, 2.14 (I) |
| SC- 090 | 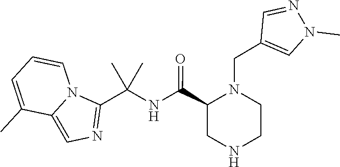 | (S)-1-((1-methyl-1H-pyra- zol-4-yl)methyl)-N-(2-(8- methylimidazo[1,5-a]pyri- din-3-yl)propan-2-yl)pipera- zine-2-carboxamide | INT- 109 | 396, 1.86 (I) |
| SC- 091 | 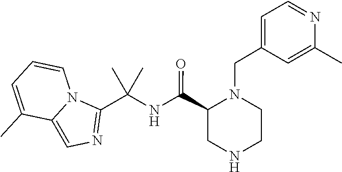 | (S)-N-(2-(8-methylimid- azo[1,5-a]pyridin-3-yl)pro- pan-2-yl)-1-((2-methylpyri- din-4-yl)methyl)piperazine- 2-carboxamide | INT- 110 | 407, 1.95 (I) |
| SC- 092 | 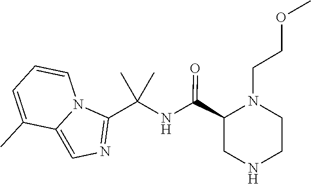 | (S)-1-(2-methoxyethyl)-N- (2-(8-methylimidazo[1,5- a]pyridin-3-yl)propan-2- yl)piperazine-2-carbox- amide | INT- 111 | 360, 3.32 (E) |
| SC- 093 | 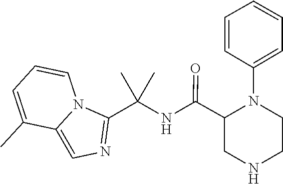 [SMILES: Cc1cccn2c(C(C)(C)NC(=O)C3CNCCN3c3ccccc3)ncc12] | rac-N-(2-(8-methylimid- azo[1,5-a]pyridin-3-yl)pro- pan-2-yl)-1-phenylpipera- zine-2-carboxamide | INT- 137 | 378, 1.93 (F) |
| SC- 094 | 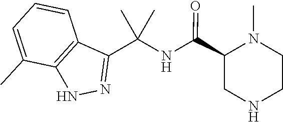 [SMILES: Cc1cccc2c(C(C)(C)NC(=O)[C@@H]3CNCCN3C)n[nH]c12] | (S)-1-methyl-N-(2-(7-me- thyl-1H-indazol-3-yl)pro- pan-2-yl)piperazine-2-car- boxamide | INT- 138 | 316, 1.92 (I) |
| SC- 095 | 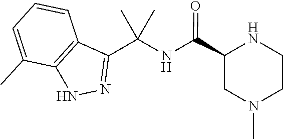 [SMILES: Cc1cccc2c(C(C)(C)NC(=O)[C@@H]3CN(C)CCN3)n[nH]c12] | (S)-4-methyl-N-(2-(7-me- thyl-1H-indazol-3-yl)pro- pan-2-yl)piperazine-2-car- boxamide | INT- 139 | 316, 1.96 (I) |
| SC- 096 | 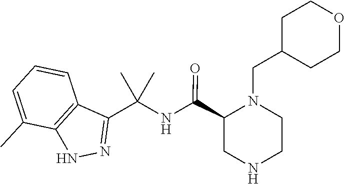 [SMILES: Cc1cccc2c(C(C)(C)NC(=O)[C@@H]3CNCCN3CC3CCOCC3)n[nH]c12] | (S)-N-(2-(7-methyl-1H-in- dazol-3-yl)propan-2-yl)-1- ((tetrahydro-2H-pyran-4- yl)methyl)piperazine-2-car- boxamide | INT- 112 | 400, 1.97 (I) |
| SC- 097 | 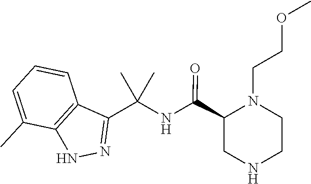 | (S)-1-(2-methoxyethyl)-N- (2-(7-methyl-1H-indazol-3- yl)propan-2-yl)piperazine-2- carboxamide | INT- 113 | 360, 2.02 (I) |
| SC- 098 | 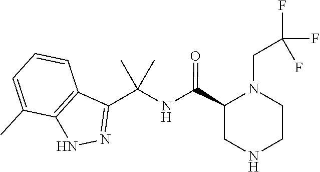 [SMILES: Cc1cccc2c(C(C)(C)NC(=O)[C@@H]3CNCCN3CC(F)(F)F)n[nH]c12] | (S)-N-(2-(7-methyl-1H-in- dazol-3-yl)propan-2-yl)-1- (2,2,2-trifluoroethyl)pipera- zine-2-carboxamide | INT- 140 | 384, 1.92 (I) |
| SC- 099 | 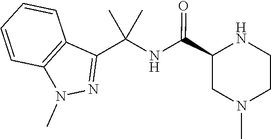 [SMILES: CN1CCN[C@H](C(=O)NC(C)(C)c2nn(C)c3ccccc23)C1] | (S)-4-methyl-N-(2-(1-me- thyl-1H-indazol-3-yl)pro- pan-2-yl)piperazine-2-car- boxamide | INT- 141 | 316, 2.00 (C) |
| SC- 100 | 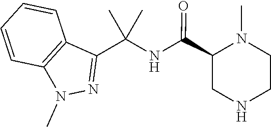 [SMILES: CN1CCNC[C@H]1C(=O)NC(C)(C)c1nn(C)c2ccccc12] | (S)-1-methyl-N-(2-(1-me- thyl-1H-indazol-3-yl)pro- pan-2-yl)piperazine-2-car- boxamide | INT- 142 | 316, 1.87 (C) |
| SC- 101 | 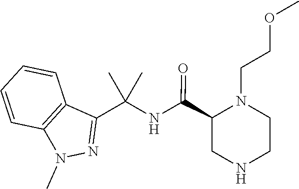 [SMILES: COCCN1CCNC[C@H]1C(=O)NC(C)(C)c1nn(C)c2ccccc12] | (S)-1-(2-methoxyethyl)-N- (2-(1-methyl-1H-indazol-3- yl)propan-2-yl)piperazine-2- carboxamide | INT- 115 | 360, 1.94 (I) |
| SC- 102 | 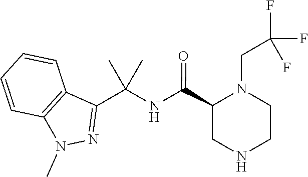 [SMILES: Cn1nc(C(C)(C)NC(=O)[C@@H]2CNCCN2CC(F)(F)F)c2ccccc21] | (S)-N-(2-(1-methyl-1H-in- dazol-3-yl)propan-2-yl)-1- (2,2,2-trifluoroethyl)pipera- zine-2-carboxamide | INT- 116 | 384, 1.97 (F) |
| SC- 103 | 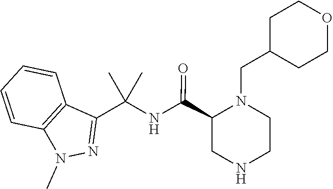 [SMILES: Cn1nc(C(C)(C)NC(=O)[C@@H]2CNCCN2CC2CCOCC2)c2ccccc21] | (S)-N-(2-(1-methyl-1H-in- dazol-3-yl)propan-2-yl)-1- ((tetrahydro-2H-pyran-4- yl)methyl)piperazine-2-car- boxamide | INT- 117 | 400, 1.97 (I) |
| SC- 104 | 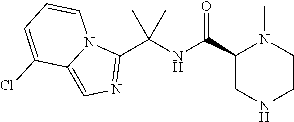 [SMILES: CN1CCNC[C@H]1C(=O)NC(C)(C)c1ncc2c(Cl)cccn12] | (S)-N-(2-(8-chloroimid- azo[1,5-a]pyridin-3-yl)pro- pan-2-yl)-1-methylpipera- zine-2-carboxamide | INT- 118 | 334 [M − H]⁻, 1.85 (I) |
| SC- 105 | 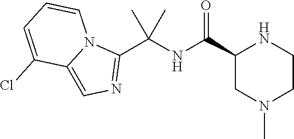 [SMILES: CN1CCN[C@H](C(=O)NC(C)(C)c2ncc3c(Cl)cccn23)C1] | (S)-N-(2-(8-chloroimid- azo[1,5-a]pyridin-3-yl)pro- pan-2-yl)-4-methylpipera- zine-2-carboxamide | INT- 119 | 336, 1.92 (I) |
| SC- 106 | 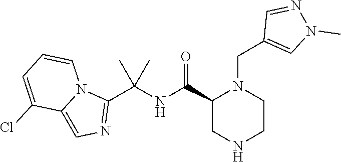 [SMILES: Cn1cc(CN2CCNC[C@H]2C(=O)NC(C)(C)c2ncc3c(Cl)cccn23)cn1] | (S)-N-(2-(8-chloroimid- azo[1,5-a]pyridin-3-yl)pro- pan-2-yl)-1-((1-methyl-1H- pyrazol-4-yl)methyl)pipera- zine-2-carboxamide | INT- 120 | 416, 1.93 (I) |
| SC- 107 | 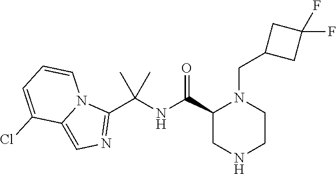 [SMILES: CC(C)(NC(=O)[C@@H]1CNCCN1CC1CC(F)(F)C1)c1ncc2c(Cl)cccn12] | (S)-N-(2-(8-chloroimid- azo[1,5-a]pyridin-3-yl)pro- pan-2-yl)-1-((3,3-difluoro- cyclobutyl)methyl)pipera- zine-2-carboxamide | INT- 121 | 426, 2.21 (I) |
| SC- 108 | 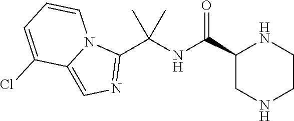 [SMILES: CC(C)(NC(=O)[C@@H]1CNCCN1)c1ncc2c(Cl)cccn12] | (S)-N-(2-(8-chloroimid- azo[1,5-a]pyridin-3-yl)pro- pan-2-yl)piperazine-2-car- boxamide | INT- 122 | 322, 1.74 (I) |
| SC- 109 | 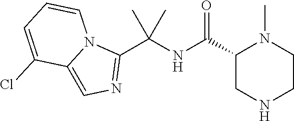 [SMILES: CN1CCNC[C@@H]1C(=O)NC(C)(C)c1ncc2c(Cl)cccn12] | (R)-N-(2-(8-chloroimid- azo[1,5-a]pyridin-3-yl)pro- pan-2-yl)-1-methylpipera- zine-2-carboxamide | INT- 123 | 336, 1.87 (I) |
| SC- 110 | 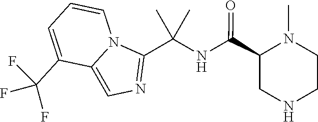 [SMILES: CN1CCNC[C@H]1C(=O)NC(C)(C)c1ncc2c(C(F)(F)F)cccn12] | (S)-1-methyl-N-(2-(8-(tri- fluoromethyl)imidazo[1,5- a]pyridin-3-yl)propan-2- yl)piperazine-2-carbox- amide | INT- 124 | 370, 1.99 (I) |
| SC- 111 | 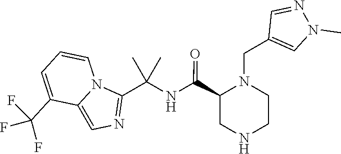 [SMILES: Cn1cc(CN2CCNC[C@H]2C(=O)NC(C)(C)c2ncc3c(C(F)(F)F)cccn23)cn1] | (S)-1-((1-methyl-1H-pyra- zol-4-yl)methyl)-N-(2-(8- (trifluoromethyl)imid- azo[1,5-a]pyridin-3-yl)pro- pan-2-yl)piperazine-2-car- boxamide | INT- 125 | 450, 2.06 (I) |
| SC- 112 | 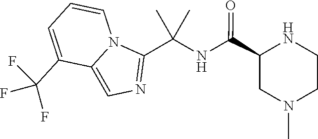 [SMILES: CN1CCN[C@H](C(=O)NC(C)(C)c2ncc3c(C(F)(F)F)cccn23)C1] | (S)-4-methyl-N-(2-(8-(tri- fluoromethyl)imidazo[1,5- a]pyridin-3-yl)propan-2- yl)piperazine-2-carbox- amide | INT- 126 | 370, 2.04 (I) |
| SC- 113 | 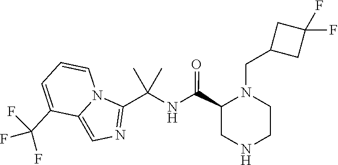 [SMILES: CC(C)(NC(=O)[C@@H]1CNCCN1CC1CC(F)(F)C1)c1ncc2c(C(F)(F)F)cccn12] | (S)-1-((3,3-difluorocyclobu- tyl)methyl)-N-(2-(8-(trifluo- romethyl)imidazo[1,5-a]pyr- idin-3-yl)propan-2-yl)piper- azine-2-carboxamide | INT- 127 | 458 [M − H]⁻, 2.29 (I) |
| SC- 114 | 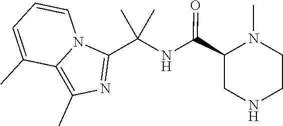 [SMILES: Cc1cccn2c(C(C)(C)NC(=O)[C@@H]3CNCCN3C)nc(C)c12] | (S)-N-(2-(1,8-dimethylimid- azo[1,5-a]pyridin-3-yl)pro- pan-2-yl)-1-methylpipera- zine-2-carboxamide | INT- 128 | 330, 4.32 (A) |
| SC- 115 | 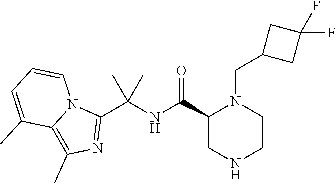 [SMILES: Cc1cccn2c(C(C)(C)NC(=O)[C@@H]3CNCCN3CC3CC(F)(F)C3)nc(C)c12] | (S)-1-((3,3-difluorocyclobu- tyl)methyl)-N-(2-(1,8-dime- thylimidazo[1,5-a]pyridin-3- yl)propan-2-yl)piperazine-2- carboxamide | INT- 129 | 420, 2.20 (F) |
| SC- 116 | 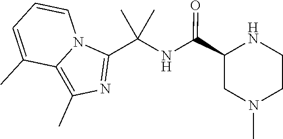 [SMILES: Cc1cccn2c(C(C)(C)NC(=O)[C@@H]3CN(C)CCN3)nc(C)c12] | (S)-N-(2-(1,8-dimethylimid- azo[1,5-a]pyridin-3-yl)pro- pan-2-yl)-4-methylpipera- zine-2-carboxamide | INT- 130 | 330, 2.33 (A) |
| SC- 117 | 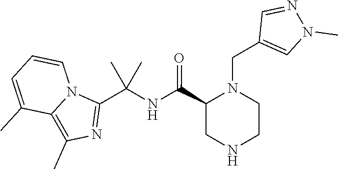 | (S)-N-(2-(1,8-dimethylimid- azo[1,5-a]pyridin-3-yl)pro- pan-2-yl)-1-((1-methyl-1H- pyrazol-4-yl)methyl)pipera- zine-2-carboxamide | INT- 131 | 410, 1.94 (I) |
| SC- 118 | 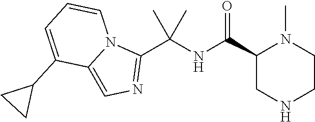 [SMILES: CN1CCNC[C@H]1C(=O)NC(C)(C)c1ncc2c(C3CC3)cccn12] | (S)-N-(2-(8-cyclopropylim- idazo[1,5-a]pyridin-3- yl)propan-2-yl)-1-methylpi- perazine-2-carboxamide | INT- 132 | 342, 1.98 (I) |
| SC- 119 | 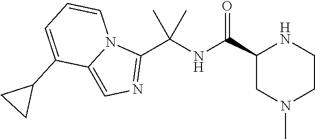 [SMILES: CN1CCN[C@H](C(=O)NC(C)(C)c2ncc3c(C4CC4)cccn23)C1] | (S)-N-(2-(8-cyclopropylim- idazo[1,5-a]pyridin-3- yl)propan-2-yl)-4-methylpi- perazine-2-carboxamide | INT- 133 | 342, 2.01 (I) |
| SC- 120 | 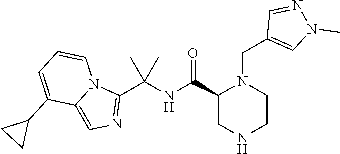 [SMILES: Cn1cc(CN2CCNC[C@H]2C(=O)NC(C)(C)c2ncc3c(C4CC4)cccn23)cn1] | (S)-N-(2-(8-cyclopropylim- idazo[1,5-a]pyridin-3- yl)propan-2-yl)-1-((1-me- thyl-1H-pyrazol-4-yl)me- thyl)piperazine-2-carbox- amide | INT- 134 | 422, 2.01 (I) |
| SC- 121 | 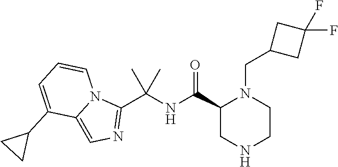 [SMILES: CC(C)(NC(=O)[C@@H]1CNCCN1CC1CC(F)(F)C1)c1ncc2c(C3CC3)cccn12] | (S)-N-(2-(8-cyclopropylim- idazo[1,5-a]pyridin-3- yl)propan-2-yl)-1-((3,3- difluorocyclobutyl)me- thyl)piperazine-2-carbox- amide | INT- 135 | 432, 2.25 (I) |
| SC- 124 | 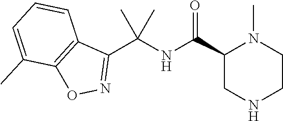 [SMILES: Cc1cccc2c(C(C)(C)NC(=O)[C@@H]3CNCCN3C)noc12] | (S)-1-methyl-N-(2-(7- methylbenzo[d]isoxazol-3- yl)propan-2-yl)piperazine-2- carboxamide | INT- 167 | 317, 0.49 (O) |
| SC- 125 | 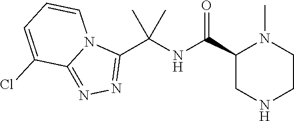 [SMILES: CN1CCNC[C@H]1C(=O)NC(C)(C)c1nnc2c(Cl)cccn12] | (S)-N-(2-(8-chloro- [1,2,4]triazolo[4,3-a]pyri- din-3-yl)propan-2-yl)-1- methylpiperazine-2-carbox- amide | INT- 162 | 337, 0.29 (O) |
| SC- 126 | 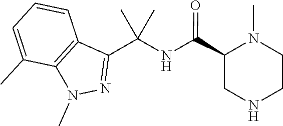 [SMILES: Cc1cccc2c(C(C)(C)NC(=O)[C@@H]3CNCCN3C)nn(C)c12] | (S)-N-(2-(1,7-dimethyl-1H- indazol-3-yl)propan-2-yl)-1- methylpiperazine-2-carbo- xamide | INT- 169 | 330, 0.48 (O) |
| SC- 127 | 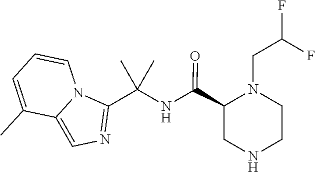 [SMILES: Cc1cccn2c(C(C)(C)NC(=O)[C@@H]3CNCCN3CC(F)F)ncc12] | (S)-1-(2,2-difluoroethyl)-N- (2-(8-methylimidazo[1,5- al]pyridin-3-yl)propan-2- yl)piperazine-2-carboxa- mide | INT- 144 | 366, 2.88 (N) |
| SC- 128 | 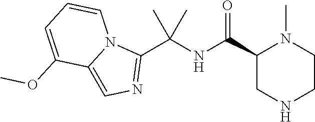 [SMILES: COc1cccn2c(C(C)(C)NC(=O)[C@@H]3CNCCN3C)ncc12] | (S)-N-(2-(8-methoxyimid- azo[1,5-a]pyridin-3-yl)pro- pan-2-yl)-1-methylpipera- zine-2-carboxamide | INT- 168 | 332, 0.29 (O) |
| SC- 129 | 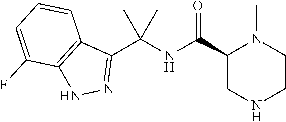 [SMILES: CN1CCNC[C@H]1C(=O)NC(C)(C)c1n[nH]c2c(F)cccc12] | (S)-N-(2-(7-fluoro-1H-inda- zol-3-yl)propan-2-yl)-1-me- thylpiperazine-2-carboxa- mide | INT- 171 | 320, 0.43 (O) |

### Synthesis of SC-122: (S)—N-(2-(7-methyl-1H-indazol-3-yl)propan-2-yl)-1-(oxetan-3-ylmethyl)piperazine-2-carboxamide

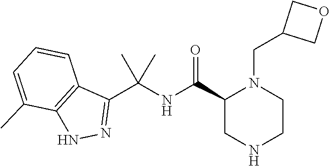 [SMILES: Cc1cccc2c(C(C)(C)NC(=O)[C@@H]3CNCCN3CC3COC3)n[nH]c12]

To a solution of carboxamide INT-143 (110 mg, 0.22 mmol) in THF was added ethylenediamine (22 μL, 0.33 mmol) and TBAF (0.33 ml, 0.33 mmol). The tube was sealed and the mixture stirred at 80° C. for 16 h. The mixture was allowed to cool to room temperature and was diluted with EtOAc (20 mL). The mixture was washed with H₂O (10 mL) and sat. aq. NaCl solution (10 mL) and the organic layer dried over anhyd. Na₂SO₄ and concentrated under reduced pressure. The residue was purified by preparative HPLC (YMC-Actus Triart C18 (250×20 mm, 5μ), elution with 20 mM NH₄HCO₃ in H₂O/MeCN (80:20-5:95) at ambient temperature) to obtain the title compound (40 mg, 49%). LCMS m/z=370 [M+H]⁺; ¹H NMR (400 MHz, DMSO-d₆) δ 12.78 (s, 1H), 8.28 (s, 1H), 7.66 (d, 1H), 7.08 (d, 1H), 6.95 (t, 1H), 4.60-4.57 (m, 1H), 4.55-4.51 (m, 1H), 4.26-4.23 (m, 1H), 4.13-4.10 (m, 1H), 3.20-3.17 (m, 1H), 2.83-2.74 (m, 2H), 2.70-2.66 (m, 2H), 2.61-2.57 (m, 2H), 2.49 (s, 4H), 2.37-2.32 (m, 1H), 2.10-2.07 (m, 1H), 1.95-1.91 (m, 1H), 1.76 (m, 6H).

### Synthesis of SC-123: (S)—N-(2-(1-methyl-1H-indazol-3-yl)propan-2-yl)-1-(oxetan-3-ylmethyl)piperazine-2-carboxamide

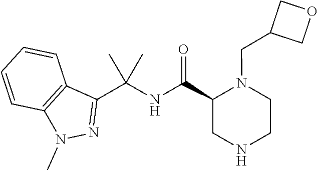 [SMILES: Cn1nc(C(C)(C)NC(=O)[C@@H]2CNCCN2CC2COC2)c2ccccc21]

To a solution of carboxamide INT-136 (100 mg, 0.20 mmol) in MeOH (5 ml) was added Pd/C (50 mg) and the reaction mixture was stirred under hydrogen atmosphere for 2 h at room temperature. The mixture was filtered through a pad of celite, washed with MeOH and concentrated under reduced pressure. The residue was purified by preparative HPLC (XBRIDGE C18 (250×19 mm, 10μ), elution with 20 mM NH₄HCO₃ in H₂O/MeOH (80:20-5:95) at ambient temperature) to obtain the title compound (13 mg, 18%). LCMS m/z=372 [M+H]⁺; ¹H NMR (400 MHz, DMSO-d₆) δ 8.20 (s, 1H), 7.85 (d, 1H), 7.55 (d, 1H), 7.35 (t, 1H), 7.06 (t, 1H), 4.60-4.52 (m, 2H), 4.24 (t, 1H), 4.11 (t, 1H), 3.97 (s, 3H), 3.20-3.15 (m, 1H), 2.79-2.54 (m, 6H), 2.37-2.32 (m, 1H), 1.95-1.90 (m, 3H), 1.74-1.72 (m, 6H).

### Biological Activity:

hSSTR4 cAMP Assay:

cAMP accumulation assay using a CHO-K1 human SSTR4 Gi cell line.

Quantification of intracellular cAMP was performed using the LANCE Ultra cAMP assay kit from Revvity according to the manufacturer's instructions. CHO-K1 human SSTR4 Gi cell were either seeded in a density of 3K cells per well into a 384 well plate and incubated at 37° C. and 5% CO₂ for 18-20 h or a stock of frozen cells were defrosted and seeded in a density of 5K cells per well without any further cultivation directly prior to the assay in a total volume of 5 μL in stimulation buffer. For pre-cultured cells, medium was replaced with 5 μL stimulation buffer before the assay. Cells were then stimulated with forskolin (final concentration 10 μM) and test compound (SSTR4 agonist) in increasing concentrations in duplicates for 30 min at 25° C. After stimulation, cells were lysed by addition of 10 μL of detection buffer (provided with the kit) and cell lysates were further diluted to ensure final cAMP concentrations fitted well into the dynamic range of the test kit. 10 μL of the diluted lysates were then added to respective wells of an OptiPlate-384 microplate. Thereafter, 5 μL of 4× Eu-cAMP tracer working solution was added, followed by addition of 5 μL of ULight-anti-cAMP working solution. The plate was then incubated for 1 h at 25° C. before reading on a TR-FRET microplate instrument. Data were normalized to cAMP high (forskolin only) and low control (forskolin+100 nM SST14) before fitting to a 4-Paramter logistic function.

In-vitro functional agonism data (EC₅₀ at hSSTR4) of the exemplified compounds is given in the table below. All data was generated according to the cAMP assay conditions described above in at least duplicate and results are averaged and rounded to two significant figures.

| Example | hSSTR4 EC₅₀ nM |
|---|---|
| SC-001 | 13 |
| SC-002 | 1.1 |
| SC-003 | 3.3 |
| SC-004 | 7.4 |
| SC-005 | 2.3 |
| SC-006 | 3.7 |
| SC-007 | 2.2 |
| SC-008 | 2.9 |
| SC-009 | 3400 |
| SC-010 | 3500 |
| SC-011 | 1300 |
| SC-012 | 6600 |
| SC-013 | 350 |
| SC-014 | 3.1 |
| SC-015 | 9.5 |
| SC-016 | 0.44 |
| SC-017 | 0.20 |
| SC-018 | 1.2 |
| SC-019 | 0.58 |
| SC-020 | 0.27 |
| SC-021 | 3.1 |
| SC-022 | 0.09 |
| SC-023 | 9.0 |
| SC-024 | 0.46 |
| SC-025 | 0.05 |
| SC-026 | 0.12 |
| SC-027 | 0.08 |
| SC-028 | 0.17 |
| SC-029 | 0.11 |
| SC-030 | 0.48 |
| SC-031 | 12 |
| SC-032 | 0.23 |
| SC-033 | 0.38 |
| SC-034 | 0.25 |
| SC-035 | 0.35 |
| SC-036 | 0.09 |
| SC-037 | 1.7 |
| SC-038 | 0.60 |
| SC-039 | 1.4 |
| SC-040 | 0.39 |
| SC-041 | 0.22 |
| SC-042 | 1.3 |
| SC-043 | 350 |
| SC-044 | 6.1 |
| SC-045 | 1.4 |
| SC-046 | 2.8 |
| SC-047 | 0.45 |
| SC-048 | 9.2 |
| SC-049 | 3300 |
| SC-050 | 8200 |
| SC-051 | 990 |
| SC-052 | 22 |
| SC-053 | 520 |
| SC-054 | 17 |
| SC-055 | 8400 |
| SC-056 | 3000 |
| SC-057 | 20 |
| SC-058 | 1700 |
| SC-059 | 130 |
| SC-060 | 1500 |
| SC-061 | 1700 |
| SC-062 | 220 |
| SC-063 | 930 |
| SC-064 | 13000 |
| SC-065 | 1300 |
| SC-066 | 1600 |
| SC-067 | 2400 |
| SC-068 | 590 |
| SC-069 | 300 |
| SC-070 | 82 |
| SC-071 | 540 |
| SC-072 | 1200 |
| SC-073 | 4800 |
| SC-074 | 2600 |
| SC-075 | 620 |
| SC-076 | 620 |
| SC-077 | >1000 |
| SC-078 | 6500 |
| SC-079 | 15000 |
| SC-080 | >10000 |
| SC-081 | >10000 |
| SC-082 | 5400 |
| SC-083 | 6700 |
| SC-084 | >10000 |
| SC-085 | 0.11 |
| SC-086 | 0.16 |
| SC-087 | 0.27 |
| SC-088 | 0.79 |
| SC-089 | 0.55 |
| SC-090 | 1.8 |
| SC-091 | 0.06 |
| SC-092 | 1.1 |
| SC-093 | 0.86 |
| SC-094 | 0.21 |
| SC-095 | 2.9 |
| SC-096 | 0.46 |
| SC-097 | 1.2 |
| SC-098 | 0.07 |
| SC-099 | 21 |
| SC-100 | 0.91 |
| SC-101 | 5.9 |
| SC-102 | 0.84 |
| SC-103 | 0.22 |
| SC-104 | 0.90 |
| SC-105 | 31 |
| SC-106 | 1.5 |
| SC-107 | 0.76 |
| SC-108 | 19 |
| SC-109 | 1600 |
| SC-110 | 36 |
| SC-111 | 94 |
| SC-112 | 490 |
| SC-113 | 26 |
| SC-114 | 0.38 |
| SC-115 | 0.34 |
| SC-116 | 32 |
| SC-117 | 1.6 |
| SC-118 | 15 |
| SC-119 | 540 |
| SC-120 | 53 |
| SC-121 | 16 |
| SC-122 | 1.2 |
| SC-123 | 7.1 |
| SC-124 | 4.7 |
| SC-125 | 1109 |
| SC-126 | 2.6 |
| SC-127 | 1.1 |
| SC-128 | 3.8 |
| SC-129 | 2.8 |

### COMPARATIVE EXAMPLES

As demonstrated by the comparative experimental data below, methylpiperazinyl compounds exhibit consistently superior potency at the SSTR4 receptor compared to their morpholinyl matched molecular pairs (from WO 2016 075240). All data was generated according to the cAMP assay conditions described above.

| | | | | | hSSTR4 |
|---|---|---|---|---|---|
| Exam- | | hSSTR4 | Comparative | | EC₅₀ |
| ple | Structure | EC₅₀ nM | Example | Structure | nM |
| SC- 016 | 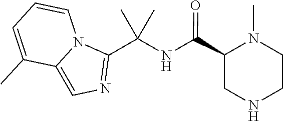 [SMILES: Cc1cccn2c(C(C)(C)NC(=O)[C@@H]3CNCCN3C)ncc12] | 0.44 | WO 2016 075240 Ex.95 | 368 | 12 |
| SC- 094 | 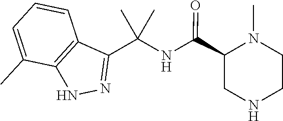 [SMILES: Cc1cccc2c(C(C)(C)NC(=O)[C@@H]3CNCCN3C)n[nH]c12] | 0.21 | WO 2016 075240 Ex.57 | 370 | 1.3 |
| SC- 104 | 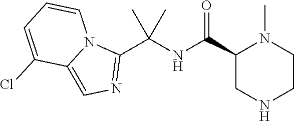 [SMILES: CN1CCNC[C@H]1C(=O)NC(C)(C)c1ncc2c(Cl)cccn12] | 0.90 | WO 2016 075240 Ex.96 | 372 | 13 |
| SC- 124 | 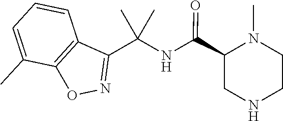 [SMILES: Cc1cccc2c(C(C)(C)NC(=O)[C@@H]3CNCCN3C)noc12] | 4.7 | WO 2016 075240 Ex.53 | 374 | 46 |
| SC- 126 | 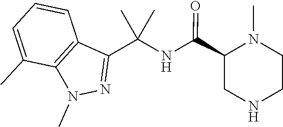 [SMILES: Cc1cccc2c(C(C)(C)NC(=O)[C@@H]3CNCCN3C)nn(C)c12] | 2.6 | WO 2016 075240 Ex.83 | 376 | 15 |
| SC- 129 | 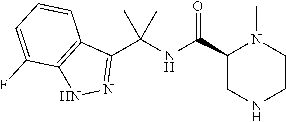 [SMILES: CN1CCNC[C@H]1C(=O)NC(C)(C)c1n[nH]c2c(F)cccc12] | 2.8 | WO 2016 075240 Ex.87 | 378 | 34 |
| SC- 125 | 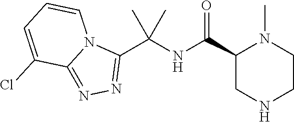 [SMILES: CN1CCNC[C@H]1C(=O)NC(C)(C)c1nnc2c(Cl)cccn12] | 1109 | (S)-N-(2-(8- chloro-[1,2,4]tria- zolo[4,3-a]pyri- din-3-yl)propan- 2-yl)morpholine- 2-carboxamide | 380 | >10000 |
| SC- 128 | 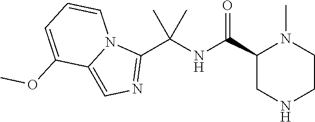 [SMILES: COc1cccn2c(C(C)(C)NC(=O)[C@@H]3CNCCN3C)ncc12] | 3.8 | WO 2016 075240 Ex.99 | 382 | 107 |

1. A compound according to general formula (I): 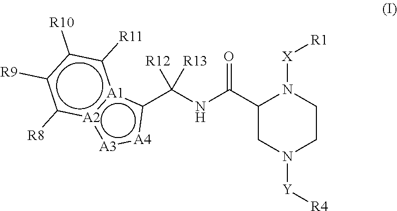
wherein
A1 represents C or N;
A2 represents C or N;
A3 represents CR7, N, NR7, or O;
A4 represents CH, or N;
with the proviso that at least one of A1, A2 and A4 represents N, or at least A3 represents N or NR7;
R1 and R4 independently of one another represent
—H;
—C₁₋₆-alkyl unsubstituted or substituted with one, two, three or four substituents independently of one another selected from —F, —OH, —OCH₃ and —OCF₃;
—C₃₋₈-cycloalkyl, unsubstituted or substituted with one, two, three or four substituents independently of one another selected from —F, —OH, —C₁₋₆-alkyl and —O—C₁₋₆-alkyl, wherein in each case alkyl is unsubstituted or substituted with one, two or three —F;
—C₂₋₇-heterocycloalkyl, unsubstituted or substituted with one, two, three or four substituents independently of one another selected from —F, —OH, —C₁₋₆-alkyl and —O—C₁₋₆-alkyl, wherein in each case alkyl is unsubstituted or substituted with one, two or three —F;
-aryl, unsubstituted or substituted with one, two, three or four substituents independently of one another selected from —F, —Cl, —OH, —C₁₋₆-alkyl and —O—C₁₋₆-alkyl, wherein in each case alkyl is unsubstituted or substituted with one, two or three —F; or
-heteroaryl, unsubstituted or substituted with one, two, three or four substituents independently of one another selected from —F, —Cl, —OH, -cyclopropyl unsubstituted, —C₁₋₆-alkyl and —O—C₁₋₆-alkyl, wherein in each case alkyl is unsubstituted or substituted with one, two or three —F;
X represents a bond, —C(═O)— or —CR2R3-;
Y represents a bond, —C(═O)—, —C(═O)CH₂—, or —CR5R6-;
R2, R3, R5 and R6 independently of one another represent
—H; or
—C₁₋₆-alkyl unsubstituted or substituted with one, two, three or four substituents independently of one another selected from —F, —OH, and —OCH₃;
R7 represents
—H;
—F or —Cl;
—C₁₋₆-alkyl or —O—C₁₋₆-alkyl, wherein in each case alkyl is unsubstituted or substituted with one, two or three —F; or
—C₃₋₈-cycloalkyl, —O—C₃₋₈-cycloalkyl, or —CH₂—C₃₋₈-cycloalkyl, wherein in each case cycloalkyl is unsubstituted or substituted with one, two or three —F;
with the proviso that when A3 represents NR7, R7 represents
—H;
—C₁₋₆-alkyl unsubstituted or substituted with one, two or three —F; or
—C₃₋₈-cycloalkyl or —CH₂—C₃₋₈-cycloalkyl, wherein in each case cycloalkyl is unsubstituted or substituted with one, two or three —F;
R8 represents
—H;
—F or —Cl;
—C₁₋₆-alkyl or —O—C₁₋₆-alkyl, wherein in each case alkyl is unsubstituted or substituted with one, two, three or four substituents independently of one another selected from —F, —OH, and —OCH₃; or
—C₃₋₄-cycloalkyl or —CH₂—C₃₋₄-cycloalkyl, wherein in each case cycloalkyl is unsubstituted or substituted with one, two, three or four substituents independently of one another selected from —F, —OH, —OCH₃, and —C₁₋₆-alkyl unsubstituted or substituted with one, two or three —F;
R9, R10 and R11 independently of one another represent —H or —F;
R12 and R13 independently of one another represent —H or —C₁₋₆-alkyl unsubstituted or substituted with one, two or three —F; or R12 and R13 together with the carbon atom to which they are attached form C₃₋₆-cycloalkyl unsubstituted or substituted with one, two or three —F;
or a physiologically acceptable salt thereof.

2. The compound according to claim 1, which has any one of general formulas (II-a) to (II-h):

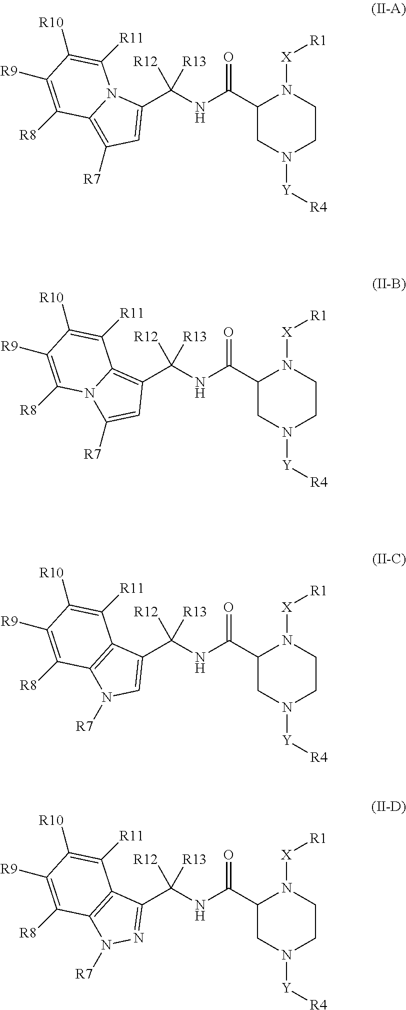

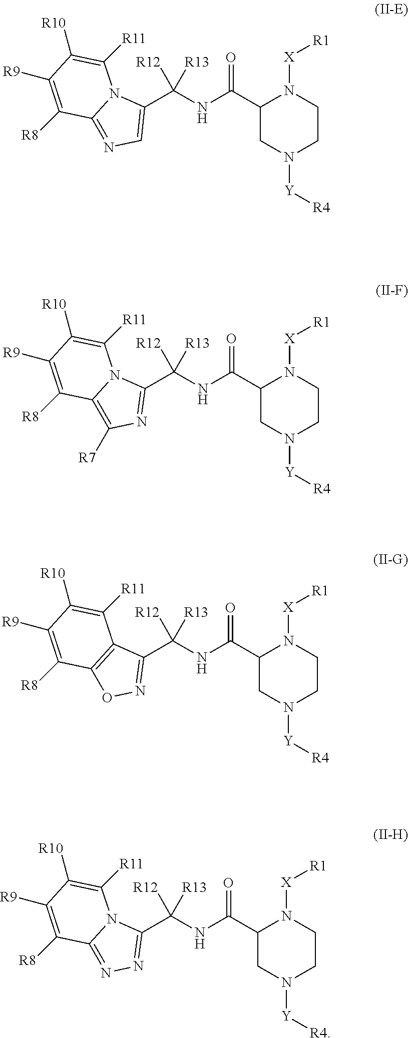

3. The compound according to claim 1, wherein X represents a bond, —C(═O)—, —CH₂—, or —CH(CH₃)—.

4. The compound according to claim 1, wherein R1 represents
—H;
—CH₃, —CH₂CH₃, —CH(CH₃)₂, —CH₂CH(CH₃)₂, —CH₂CHF₂, —CH₂CF₃, —CH₂CF(CH₃)₂, —CH₂CF₂CH₃, —CH₂CH₂OCH₃, —CH₂C(CH₃)₂OH, or —CH₂C(CH₃)₂CH₂OCH₃;
-cyclopropyl, -cyclobutyl, or -cyclohexyl; in each case unsubstituted or substituted with one or two substituents independently of one another selected from —F and —CF₃;
-oxetanyl or -tetrahydropyranyl; in each case unsubstituted or monosubstituted with —CH₃;
-phenyl unsubstituted or monosubstituted with —F or —CF₃; or
-pyrazolyl, -pyridinyl, -pyrimidinyl, -pyridazinyl, or -pyrazinyl; in each case unsubstituted or monosubstituted with —CH₃, —CF₃, or -cyclopropyl unsubstituted.

5. The compound according to claim 1, wherein Y represents a bond, —C(═O)—, —C(═O)CH₂—, —CH₂—, or —CH(CH₃)—.

6. The compound according to claim 1, wherein R4 represents
—H;
—CH₃, CH₂CH₃, —CH₂CH₂CH₃, —CH(CH₃)₂, —CH₂CH(CH₃)₂, —CH₂CHF₂, —CH₂CF₃, or —CH₂CH₂OCH₃;
-phenyl unsubstituted;
-cyclopropyl, cyclobutyl or cyclohexyl; in each case unsubstituted or substituted with one or two —F;
-oxetanyl or -tetrahydropyranyl; in each case unsubstituted; or
-pyrazolyl, -oxazolyl, -pyridyl; in each case unsubstituted or monosubstituted with —CH₃, or —CH(CH₃)₂.

7. The compound according to claim 1, wherein R7 represents —H or —CH₃.

8. The compound according to claim 1, wherein R8 represents —H, —F, —Cl, —CH₃, —CF₃, —OCH₃, or -cyclopropyl unsubstituted.

9. The compound according to claim 1, wherein R9, R10 and R11 each represent —H; and/or wherein R12 and R13 each represent —CH₃.

10. The compound according to claim 1, which is selected from the group consisting of
00 rac-N-(2-(8-methylimidazo [1,5-a] pyridin-3-yl)propan-2-yl)-1-(pyrimidin-2-ylmethyl)piperazine-2-carboxamide;
002 rac-1-isopropyl-N-(2-(8-methylimidazo [1,5-a] pyridin-3-yl)propan-2-yl)-piperazine-2-carboxamide;
003 rac-N-(2-(8-methylimidazo [1,5-a] pyridin-3-yl)propan-2-yl)-1-(pyridin-2-yl-methyl)piperazine-2-carboxamide;
004 rac-N-(2-(8-methylimidazo [1,5-a] pyridin-3-yl)propan-2-yl)-1-(pyridazin-3-ylmethyl)piperazine-2-carboxamide;
rac-N-(2-(8-methylimidazo [1,5-a] pyridin-3-yl)propan-2-yl)-1-(pyrimidin-5-ylmethyl)piperazine-2-carboxamide;
006 rac-1- ((1-methyl-1H-pyrazol-3-yl)methyl)-N-(2-(8-methylimidazo [1,5-a]-pyridin-3-yl)propan-2-yl)piperazine-2-carboxamide;
rac-N-(2-(8-methylimidazo [1,5-a] pyridin-3-yl)propan-2-yl)-1-(pyrazin-2-ylmethyl)piperazine-2-carboxamide;
008 rac-N-(2-(8-methylimidazo [1,5-a] pyridin-3-yl)propan-2-yl)-1-(pyrimidin-4-ylmethyl)piperazine-2-carboxamide;
rac-1-acetyl-N-(2-(8-methylimidazo [1,5-a] pyridin-3-yl)propan-2-yl)-piperazine-2-carboxamide;
rac-1-(1-methyl-1H-pyrazole-4-carbonyl)-N-(2-(8-methylimidazo [1,5-a]-pyridin-3-yl)propan-2-yl)piperazine-2-carboxamide;
011 rac-1-(3,3-difluorocyclobutane-1-carbonyl)-N-(2-(8-methylimidazo [1,5-a]-pyridin-3-yl)propan-2-yl)piperazine-2-carboxamide;
012 rac-1-(cyclopropanecarbonyl)-N-(2-(8-methylimidazo [1,5-a] pyridin-3-yl)-propan-2-yl)piperazine-2-carboxamide;
013 rac-N-(2-(8-methylimidazo [1,5-a] pyridin-3-yl)propan-2-yl)-1-(oxetane-3-carbonyl)piperazine-2-carboxamide;
014 rac-N-(2-(8-methylimidazo [1,5-a] pyridin-3-yl)propan-2-yl)-1-(oxetan-3-yl-methyl)piperazine-2-carboxamide;
(S)-N-(2-(8-methylimidazo [1,5-a] pyridin-3-yl)propan-2-yl)piperazine-2-carboxamide;
(S)-1-methyl-N-(2-(8-methylimidazo [1,5-a] pyridin-3-yl)propan-2-yl)-016 piperazine-2-carboxamide;
(S)-N-(2-(8-methylimidazo [1,5-a] pyridin-3-yl)propan-2-yl)-1-(2,2,2-01 trifluoroethyl)piperazine-2-carboxamide;
018 (S)-1-(2-fluoro-2-methylpropyl)-N-(2-(8-methylimidazo [1,5-a] pyridin-3-yl)propan-2-yl)piperazine-2-carboxamide;
019 (S)-1-(2,2-difluoropropyl)-N-(2-(8-methylimidazo [1,5-a] pyridin-3-yl)-propan-2-yl)piperazine-2-carboxamide;
(S)-N-(2-(8-methylimidazo [1,5-a] pyridin-3-yl)propan-2-yl)-1- ((1-(trifluoro-methyl)cyclopropyl)methyl)piperazine-2-carboxamide;
021 (S)-1-(3,3-difluorocyclobutyl)-N-(2-(8-methylimidazo [1,5-a] pyridin-3-yl)-propan-2-yl)piperazine-2-carboxamide;
022 (S)-1- ((2-cyclopropylpyridin-4-yl)methyl)-N-(2-(8-methylimidazo [1,5-a]-pyridin-3-yl)propan-2-yl)piperazine-2-carboxamide;
(S)-1-(2-hydroxy-2-methylpropyl)-N-(2-(8-methylimidazo [1,5-a] pyridin-3-023 yl)propan-2-yl)piperazine-2-carboxamide;
02 (S)-1- ((4,4-difluoro-1-methylcyclohexyl)methyl)-N-(2-(8-methyl-imidazo [1,5-a] pyridin-3-yl)propan-2-yl)piperazine-2-carboxamide;
025 (S)-N-(2-(8-methylimidazo [1,5-a] pyridin-3-yl)propan-2-yl)-1-(3-(trifluoro-methyl)benzyl)piperazine-2-carboxamide;
026 (S)-1-(2-fluorobenzyl)-N-(2-(8-methylimidazo [1,5-a] pyridin-3-yl)propan-2-yl)piperazine-2-carboxamide;
027 (S)-1-(4-fluorobenzyl)-N-(2-(8-methylimidazo [1,5-a] pyridin-3-yl)propan-2-yl)piperazine-2-carboxamide;
028 (S)-N-(2-(8-methylimidazo [1,5-a] pyridin-3-yl)propan-2-yl)-1-(4-(trifluoro-methyl)benzyl)piperazine-2-carboxamide;
(S)-1-(3-fluorobenzyl)-N-(2-(8-methylimidazo [1,5-a] pyridin-3-yl)propan-2-029 yl)piperazine-2-carboxamide;
030 (S)-N-(2-(8-methylimidazo [1,5-a] pyridin-3-yl)propan-2-yl)-1- ((6-(trifluoro-methyl)pyridin-3-yl)methyl)piperazine-2-carboxamide;
031 (S)-N-(2-(8-methylimidazo [1,5-a] pyridin-3-yl)propan-2-yl)-1- ((3-methyl-oxetan-3-yl)methyl)piperazine-2-carboxamide;
032 (S)-1-(4,4-difluorocyclohexyl)-N-(2-(8-methylimidazo [1,5-a] pyridin-3-yl)-propan-2-yl)piperazine-2-carboxamide;
033 (S)-1-(cyclobutylmethyl)-N-(2-(8-methylimidazo [1,5-a] pyridin-3-yl)-propan-2-yl)piperazine-2-carboxamide;
(S)-1-isobutyl-N-(2-(8-methylimidazo [1,5-a] pyridin-3-yl)propan-2-yl)-034 piperazine-2-carboxamide;
035 (S)-1-isopropyl-N-(2-(8-methylimidazo [1,5-a] pyridin-3-yl)propan-2-yl)-piperazine-2-carboxamide;
036 (S)-N-(2-(8-methylimidazo [1,5-a] pyridin-3-yl)propan-2-yl)-1- ((2-(trifluoro-methyl)pyridin-4-yl)methyl)piperazine-2-carboxamide;
037 (S)-N-(2-(8-methylimidazo [1,5-a] pyridin-3-yl)propan-2-yl)-1- ((4-methyl-tetrahydro-2H-pyran-4-yl)methyl)piperazine-2-carboxamide;
038 (S)-N-(2-(8-methylimidazo [1,5-a] pyridin-3-yl)propan-2-yl)-1- ((2-(trifluoro-methyl)pyrimidin-5-yl)methyl)piperazine-2-carboxamide;
039 (S)-1-(3-methoxy-2,2-dimethylpropyl)-N-(2-(8-methylimidazo [1,5-a]-pyridin-3-yl)propan-2-yl)piperazine-2-carboxamide;
040 (S)-1-isopropyl-N-(2-(7-methyl-1H-indazol-3-yl)propan-2-yl)piperazine-2-carboxamide;
041 (S)-1-ethyl-N-(2-(7-methyl-1H-indazol-3-yl)propan-2-yl)piperazine-2-carboxamide;
042 (S)-N-(2-(8-methylimidazo [1,5-a] pyridin-3-yl)propan-2-yl)-1-(oxetan-3-yl-methyl)piperazine-2-carboxamide;
043 (S)-4-methyl-N-(2-(8-methylimidazo [1,5-a] pyridin-3-yl)propan-2-yl)-piperazine-2-carboxamide;
044 (S)-4-methyl-1- ((1-methyl-1H-pyrazol-4-yl)methyl)-N-(2-(8-methyl-imidazo [1,5-a] pyridin-3-yl)propan-2-yl)piperazine-2-carboxamide;
045 (S)-4-methyl-N-(2-(8-methylimidazo [1,5-a] pyridin-3-yl)propan-2-yl)-1-(2,2,2-trifluoroethyl)piperazine-2-carboxamide;
046 (S)-1,4-dimethyl-N-(2-(8-methylimidazo [1,5-a] pyridin-3-yl)propan-2-yl)-piperazine-2-carboxamide;
047 (S)-4-methyl-N-(2-(8-methylimidazo [1,5-a] pyridin-3-yl)propan-2-yl)-1-((tetrahydro-2H-pyran-4-yl)methyl)piperazine-2-carboxamide;
048 (S)-4-methyl-N-(2-(8-methylimidazo [1,5-a] pyridin-3-yl)propan-2-yl)-1-(oxetan-3-ylmethyl)piperazine-2-carboxamide;
049 (S)-N-(2-(8-methylimidazo [1,5-a] pyridin-3-yl)propan-2-yl)-4-(2,2,2-trifluoroethyl)piperazine-2-carboxamide;
050 (S)-4-(2,2-difluoroethyl)-N-(2-(8-methylimidazo [1,5-a] pyridin-3-yl)propan-2-yl)piperazine-2-carboxamide;
051 (S)-4-(2-methoxyethyl)-N-(2-(8-methylimidazo [1,5-a] pyridin-3-yl)propan-2-yl)piperazine-2-carboxamide;
052 (S)-4-isopropyl-N-(2-(8-methylimidazo [1,5-a] pyridin-3-yl)propan-2-yl)-piperazine-2-carboxamide;
053 (S)-4-cyclobutyl-N-(2-(8-methylimidazo [1,5-a] pyridin-3-yl)propan-2-yl)-piperazine-2-carboxamide;
054 (S)-4-ethyl-N-(2-(8-methylimidazo [1,5-a] pyridin-3-yl)propan-2-yl)-piperazine-2-carboxamide;
055 (S)-4-(4,4-difluorocyclohexyl)-N-(2-(8-methylimidazo [1,5-a] pyridin-3-yl)-propan-2-yl)piperazine-2-carboxamide;
056 (S)-N-(2-(8-methylimidazo [1,5-a] pyridin-3-yl)propan-2-yl)-4-(tetrahydro-2H-pyran-4-yl)piperazine-2-carboxamide;
057 (S)-N-(2-(8-methylimidazo [1,5-a] pyridin-3-yl)propan-2-yl)-4- ((tetrahydro-2H-pyran-4-yl)methyl)piperazine-2-carboxamide;
058 (S)-4-benzyl-N-(2-(8-methylimidazo [1,5-a] pyridin-3-yl)propan-2-yl)-piperazine-2-carboxamide;
059 (S)-N-(2-(8-methylimidazo [1,5-a] pyridin-3-yl)propan-2-yl)-4-(pyridin-4-yl-methyl)piperazine-2-carboxamide;
060 (S)-N-(2-(8-methylimidazo [1,5-a] pyridin-3-yl)propan-2-yl)-4-(pyridin-2-yl-methyl)piperazine-2-carboxamide;
061 (S)-N-(2-(8-methylimidazo [1,5-a] pyridin-3-yl)propan-2-yl)-4-(pyridin-3-yl-methyl)piperazine-2-carboxamide;
062 (S)-N-(2-(8-methylimidazo [1,5-a] pyridin-3-yl)propan-2-yl)-4-(oxazol-5-yl-methyl)piperazine-2-carboxamide;
063 (S)-N-(2-(8-methylimidazo [1,5-a] pyridin-3-yl)propan-2-yl)-4-(oxazol-4-yl-methyl)piperazine-2-carboxamide;
064 (S)-4- ((1-methyl-3-(trifluoromethyl)-1H-pyrazol-5-yl) methyl)-N-(2-(8-methylimidazo [1,5-a] pyridin-3-yl)propan-2-yl)piperazine-2-carboxamide;
065 (S)-N-(2-(8-methylimidazo [1,5-a] pyridin-3-yl)propan-2-yl)-4-(oxazol-2-yl-methyl)piperazine-2-carboxamide;
066 (S)-4- ((1-methyl-1H-pyrazol-3-yl)methyl)-N-(2-(8-methylimidazo [1,5-a]-pyridin-3-yl)propan-2-yl)piperazine-2-carboxamide;
067 (S)-4- ((4,4-difluorocyclohexyl)methyl)-N-(2-(8-methylimidazo [1,5-a]-pyridin-3-yl)propan-2-yl)piperazine-2-carboxamide;
068 (S)-4-(cyclobutylmethyl)-N-(2-(8-methylimidazo [1,5-a] pyridin-3-yl)-propan-2-yl)piperazine-2-carboxamide;
069 (S)-4-isobutyl-N-(2-(8-methylimidazo [1,5-a] pyridin-3-yl)propan-2-yl)-piperazine-2-carboxamide;
(S)-N-(2-(8-methylimidazo [1,5-a] pyridin-3-yl)propan-2-yl)-4-propyl-070piperazine-2-carboxamide;
071 (S)-4- ((1-methyl-1H-pyrazol-4-yl)methyl)-N-(2-(8-methylimidazo [1,5-a]-pyridin-3-yl)propan-2-yl)piperazine-2-carboxamide;
072 (S)-4- ((1-methyl-1H-pyrazol-5-yl)methyl)-N-(2-(8-methylimidazo [1,5-a]-pyridin-3-yl)propan-2-yl)piperazine-2-carboxamide;
073 (S)-4- ((1-isopropyl-1H-pyrazol-3-yl)methyl)-N-(2-(8-methylimidazo [1,5-a] pyridin-3-yl)propan-2-yl)piperazine-2-carboxamide;
074 (S)-4- ((1-isopropyl-1H-pyrazol-4-yl)methyl)-N-(2-(8-methylimidazo [1,5-a] pyridin-3-yl)propan-2-yl)piperazine-2-carboxamide;
075 (S)-N-(2-(8-methylimidazo [1,5-a] pyridin-3-yl)propan-2-yl)-4-(oxetan-3-yl)piperazine-2-carboxamide;
076 (S)-N-(2-(8-methylimidazo [1,5-a] pyridin-3-yl)propan-2-yl)-4-(oxetan-3-ylmethyl)piperazine-2-carboxamide;
077 (S)-4-(cyclopropanecarbonyl)-N-(2-(8-methylimidazo [1,5-a] pyridin-3-yl)-propan-2-yl)piperazine-2-carboxamide;
078 (S)-N-(2-(8-methylimidazo [1,5-a] pyridin-3-yl)propan-2-yl)-4-(tetrahydro-2H-pyran-4-carbonyl)piperazine-2-carboxamide;
079 (S)-N-(2-(8-methylimidazo [1,5-a] pyridin-3-yl)propan-2-yl)-4-(2-(tetra-hydro-2H-pyran-4-yl)acetyl)piperazine-2-carboxamide;
080 (S)-4-(3,3-difluorocyclobutane-1-carbonyl)-N-(2-(8-methylimidazo [1,5-a]-pyridin-3-yl)propan-2-yl)piperazine-2-carboxamide;
081 (S)-4-(1-isopropyl-1H-pyrazole-4-carbonyl)-N-(2-(8-methylimidazo [1,5-a]-pyridin-3-yl)propan-2-yl)piperazine-2-carboxamide;
082 (S)-4-(1-methyl-1H-pyrazole-5-carbonyl)-N-(2-(8-methylimidazo [1,5-a]-pyridin-3-yl)propan-2-yl)piperazine-2-carboxamide;
083 (S)-4-(1-methyl-1H-pyrazole-4-carbonyl)-N-(2-(8-methylimidazo [1,5-a]-pyridin-3-yl)propan-2-yl)piperazine-2-carboxamide;
084 (S)-4-(1-methyl-1H-pyrazole-3-carbonyl)-N-(2-(8-methylimidazo [1,5-a]-pyridin-3-yl)propan-2-yl)piperazine-2-carboxamide;
085 (S)-1-benzyl-N-(2-(8-methylimidazo [1,5-a] pyridin-3-yl)propan-2-yl)-piperazine-2-carboxamide;
086 (S)-N-(2-(8-methylimidazo [1,5-a] pyridin-3-yl)propan-2-yl)-1- ((tetrahydro-2H-pyran-4-yl)methyl)piperazine-2-carboxamide;
087 (S)-1- ((4,4-difluorocyclohexyl)methyl)-N-(2-(8-methylimidazo [1,5-a]-pyridin-3-yl)propan-2-yl)piperazine-2-carboxamide;
088 (S)-N-(2-(8-methylimidazo [1,5-a] pyridin-3-yl)propan-2-yl)-1- ((6-methyl-pyridin-3-yl)methyl)piperazine-2-carboxamide;
089 (S)-1- ((3,3-difluorocyclobutyl)methyl)-N-(2-(8-methylimidazo [1,5-a]-pyridin-3-yl)propan-2-yl)piperazine-2-carboxamide;
090 (S)-1- ((1-methyl-1H-pyrazol-4-yl)methyl)-N-(2-(8-methylimidazo [1,5-a]-pyridin-3-yl)propan-2-yl)piperazine-2-carboxamide;
091 (S)-N-(2-(8-methylimidazo [1,5-a] pyridin-3-yl)propan-2-yl)-1- ((2-methyl-pyridin-4-yl)methyl)piperazine-2-carboxamide;
092 (S)-1-(2-methoxyethyl)-N-(2-(8-methylimidazo [1,5-a] pyridin-3-yl)propan-2-yl)piperazine-2-carboxamide;
093 rac-N-(2-(8-methylimidazo [1,5-a] pyridin-3-yl)propan-2-yl)-1-phenyl-piperazine-2-carboxamide;
094 (S)-1-methyl-N-(2-(7-methyl-1H-indazol-3-yl)propan-2-yl)piperazine-2-carboxamide;
095 (S)-4-methyl-N-(2-(7-methyl-1H-indazol-3-yl)propan-2-yl)piperazine-2-carboxamide;
096 (S)-N-(2-(7-methyl-1H-indazol-3-yl)propan-2-yl)-1- ((tetrahydro-2H-pyran-4-yl)methyl)piperazine-2-carboxamide;
097 (S)-1-(2-methoxyethyl)-N-(2-(7-methyl-1H-indazol-3-yl)propan-2-yl)-piperazine-2-carboxamide;
098 (S)-N-(2-(7-methyl-1H-indazol-3-yl)propan-2-yl)-1-(2,2,2-trifluoroethyl)-piperazine-2-carboxamide;
099 (S)-4-methyl-N-(2-(1-methyl-1H-indazol-3-yl)propan-2-yl)piperazine-2-carboxamide;
100 (S)-1-methyl-N-(2-(1-methyl-1H-indazol-3-yl)propan-2-yl)piperazine-2-carboxamide;
101 (S)-1-(2-methoxyethyl)-N-(2-(1-methyl-1H-indazol-3-yl)propan-2-yl)-piperazine-2-carboxamide;
102 (S)-N-(2-(1-methyl-1H-indazol-3-yl)propan-2-yl)-1-(2,2,2-trifluoroethyl)-piperazine-2-carboxamide;
103 (S)-N-(2-(1-methyl-1H-indazol-3-yl)propan-2-yl)-1- ((tetrahydro-2H-pyran-4-yl)methyl)piperazine-2-carboxamide;
(S)-N-(2-(8-chloroimidazo [1,5-a] pyridin-3-yl)propan-2-yl)-1-methyl-104 piperazine-2-carboxamide;
105 (S)-N-(2-(8-chloroimidazo [1,5-a] pyridin-3-yl)propan-2-yl)-4-methyl-piperazine-2-carboxamide;
106 (S)-N-(2-(8-chloroimidazo [1,5-a] pyridin-3-yl)propan-2-yl)-1- ((1-methyl-1H-pyrazol-4-yl)methyl)piperazine-2-carboxamide;
107 (S)-N-(2-(8-chloroimidazo [1,5-a] pyridin-3-yl)propan-2-yl)-1- ((3,3-difluorocyclobutyl)methyl)piperazine-2-carboxamide;
108 (S)-N-(2-(8-chloroimidazo [1,5-a] pyridin-3-yl)propan-2-yl)piperazine-2-carboxamide;
109 (R)-N-(2-(8-chloroimidazo [1,5-a] pyridin-3-yl)propan-2-yl)-1-methyl-piperazine-2-carboxamide;
110 (S)-1-methyl-N-(2-(8-(trifluoromethyl)imidazo [1,5-a] pyridin-3-yl)propan-2-yl)piperazine-2-carboxamide;
111 (S)-1- ((1-methyl-1H-pyrazol-4-yl)methyl)-N-(2-(8-(trifluoromethyl)-imidazo [1,5-a] pyridin-3-yl)propan-2-yl)piperazine-2-carboxamide;
112 (S)-4-methyl-N-(2-(8-(trifluoromethyl)imidazo [1,5-a] pyridin-3-yl)propan-2-yl)piperazine-2-carboxamide;
113 (S)-1- ((3,3-difluorocyclobutyl)methyl)-N-(2-(8-(trifluoromethyl)imidazo-[1,5-a] pyridin-3-yl)propan-2-yl)piperazine-2-carboxamide;
114 (S)-N-(2-(1,8-dimethylimidazo [1,5-a] pyridin-3-yl)propan-2-yl)-1-methyl-piperazine-2-carboxamide;
115 (S)-1- ((3,3-difluorocyclobutyl)methyl)-N-(2-(1,8-dimethylimidazo [1,5-a] pyridin-3-yl)propan-2-yl)piperazine-2-carboxamide;
116 (S)-N-(2-(1,8-dimethylimidazo [1,5-a] pyridin-3-yl)propan-2-yl)-4-methyl-piperazine-2-carboxamide;
117 (S)-N-(2-(1,8-dimethylimidazo [1,5-a] pyridin-3-yl)propan-2-yl)-1- ((1-methyl-1H-pyrazol-4-yl)methyl)piperazine-2-carboxamide;
118 (S)-N-(2-(8-cyclopropylimidazo [1,5-a] pyridin-3-yl)propan-2-yl)-1-methyl-piperazine-2-carboxamide;
119 (S)-N-(2-(8-cyclopropylimidazo [1,5-a] pyridin-3-yl)propan-2-yl)-4-methyl-piperazine-2-carboxamide;
120 (S)-N-(2-(8-cyclopropylimidazo [1,5-a] pyridin-3-yl)propan-2-yl)-1- ((1-methyl-1H-pyrazol-4-yl)methyl)piperazine-2-carboxamide;
121 (S)-N-(2-(8-cyclopropylimidazo [1,5-a] pyridin-3-yl)propan-2-yl)-1- ((3,3-difluorocyclobutyl)methyl)piperazine-2-carboxamide;
122 (S)-N-(2-(7-methyl-1H-indazol-3-yl)propan-2-yl)-1-(oxetan-3-ylmethyl)-piperazine-2-carboxamide;
123 (S)-N-(2-(1-methyl-1H-indazol-3-yl)propan-2-yl)-1-(oxetan-3-ylmethyl)-piperazine-2-carboxamide;
(S)-1-methyl-N-(2-(7-methylbenzo [d] isoxazol-3-yl)propan-2-yl)piperazine-1242-carboxamide;
125 (S)-N-(2-(8-chloro-[1,2,4] triazolo [4,3-a] pyridin-3-yl)propan-2-yl)-1-methylpiperazine-2-carboxamide;
126 (S)-N-(2-(1,7-dimethyl-1H-indazol-3-yl)propan-2-yl)-1-methylpiperazine-2-carboxamide;
127 (S)-1-(2,2-difluoroethyl)-N-(2-(8-methylimidazo [1,5-a] pyridin-3-yl)propan-2-yl)piperazine-2-carboxamide;
128 (S)-N-(2-(8-methoxyimidazo [1,5-a] pyridin-3-yl)propan-2-yl)-1-methylpiperazine-2-carboxamide; or
129 (S)-N-(2-(7-fluoro-1H-indazol-3-yl)propan-2-yl)-1-methylpiperazine-2-carboxamide;
or a physiologically acceptable salt thereof.

11. A pharmaceutical composition comprising a compound according to claim 1.

12. A pharmaceutical composition comprising a compound according to claim 10.

13. A method of treating pain in a patient in need thereof, said method comprising administering to said patient an effective amount therefor of at least one compound according to claim 1.

14. A method of treating pain in a patient in need thereof, said method comprising administering to said patient an effective amount therefor of at least one compound according to claim 10.
